(19) United States
(12) Patent Application Publication
(10) Pub. No.: US 2023/0143128 A1
(43) Pub. Date: May 11, 2023

(54) CARBOHYDRATE BINDING MODULE VARIANTS

(71) Applicant: Novozymes A/S, Bagsvaerd (DK)

(72) Inventors: Kenneth Jensen, Oelsted (DK); Lars Anderson, Malmoe (SE); Svend Gunnar Kaasgaard, Skovlunde (DK); Jonatan Ulrik Fangel, Copenhagen (DK); Frank Winther Rasmussen, Roskilde (DK); Pengfei Tian, Copenhagen (DK); Christian Isak Joergensen, Bagsvaerd (DK)

(73) Assignee: Novozymes A/S, Bagsvaerd (DK)

(21) Appl. No.: 17/915,903

(22) PCT Filed: Apr. 7, 2021
(86) PCT No.: PCT/EP2021/058998

§ 371 Date: Sep. 29, 2022

(30) Foreign Application Priority Data
Apr. 8, 2020 (EP) 20168634.2
Apr. 8, 2020 (EP) PCT/EP2020/059965

(51) Int. Cl. C11D 3/386; C12N 9/42; C11D 11/00; C07K 14/37

(52) U.S. Cl. CPC C11D 3/38645; C12N 9/2437; C12Y 302/01004; C11D 11/0017; C07K 14/37; C07K 2319/50

(57) ABSTRACT

Disclosed are carbohydrate binding module variants, as well as variants of a glycoside hydrolase comprising the carbohydrate binding module variants. The variants having glycoside hydrolase activity comprising the carbohydrate binding module variants have improved stability in the presence of a protease, and are useful in detergent applications, such as laundry or dish wash.

### REFERENCE TO SEQUENCE LISTING

This application contains a Sequence Listing in computer readable form. The computer readable form is incorporated herein by reference.

### FIELD OF THE INVENTION

The present invention relates to carbohydrate binding module variants, and more preferably to Family 1 carbohydrate binding module variants.

### BACKGROUND OF THE INVENTION

Glycoside hydrolases such as cellulases generally contain a catalytic domain and one or more carbohydrate binding modules (CBM), which are joined by a linker region.

Cellulases have for many years been used in detergents due to their observed benefits in the laundry process, such as color clarification, prevent redeposition, anti-pilling/pill removal and improved whiteness, and are characterized by their ability to cleave the 1,4-beta-glycosidic linkages in cellulose molecules into smaller molecules.

In some applications a complex cellulase enzyme composition is used, where the composition comprises more than one cellulose degrading enzyme, selected among endoglucanases, cellobiohydrolases and beta-glucosidases are used, whereas other applications uses enzyme compositions mainly comprising one or more endoglucanases.

WO 1996/029397 discloses family 45 endoglucanases for detergent use. Most commercial detergent compositions comprise proteases that improve the removal of many common stains. However, proteases also degrade other proteins available in an aqueous reaction mixture, including other enzymes such as cellulases and other glycoside hydrolases. It is therefore desirable to provide glycoside hydrolases, such as cellulases and variants thereof having increased stability in the presence of proteases.

### SUMMARY OF THE INVENTION

The present invention relates to carbohydrate binding module variants, and more preferably to Family 1 carbohydrate binding module variants. The invention also relates to modified glycoside hydrolase enzymes comprising the carbohydrate binding module variants.

The invention further relates to polynucleotides and expression constructs comprising the polynucleotide; host cells comprising the polynucleotides or expression constructs and the use of such host cells for producing the carbohydrate binding module variants and glycoside hydrolase enzymes comprising the carbohydrate binding module variants of the invention.

Compositions, in particular detergent compositions, such as liquid detergent compositions, comprising the carbohydrate binding module variants and glycoside hydrolase enzymes comprising the carbohydrate binding module variants, and the use of such compositions for laundering textiles are also disclosed.

### Definitions

“a” and “an” and “the” and similar referents used in the context of describing the invention are to be construed to cover both the singular and the plural, unless otherwise indicated herein or clearly contradicted by the context.

Allelic variant: The term “allelic variant” means any of two or more alternative forms of a gene occupying the same chromosomal locus. Allelic variation arises naturally through mutation and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Anti-pilling: The term “anti-pilling” denotes removal of pills from the textile surface and/or prevention of formation of pills on the textile surface.

Carbohydrate binding module: The term “carbohydrate binding module” means the region within a carbohydrate-active enzyme that provides carbohydrate-binding affinity (Boraston et al., 2004, Biochem. J. 383: 769-781). A majority of known carbohydrate binding modules (CBMs) are contiguous amino acid sequences with a discrete fold. The carbohydrate binding module (CBM) is typically found either at the N-terminal or at the C-terminal extremity of an enzyme. Some CBMs are known to have specificity for cellulose.

Exemplary CBM families useful according to the invention are those of CBM family 1, 4, 17, 28, 30, 44, 72 and 79. Again, with reference to cazy.org/Carbohydrate-Binding-Modules, CBM Family 1 includes modules of approximately 40 residues found almost exclusively in fungi. The cellulose-binding function has been demonstrated in many cases, and appears to be mediated by three aromatic residues separated by about 10.4 angstrom and which form a flat surface. CBM family 4 includes modules of approximately 150 residues found in bacterial enzymes. Binding of these modules has been demonstrated with xylan, beta-1,3-glucan, beta-1,3-1,4-glucan, beta-1,6-glucan and amorphous cellulose but not with crystalline cellulose. CBM family 17 includes modules of approximately 200 residues. Binding to amorphous cellulose, cellooligosaccharides and derivatized cellulose has been demonstrated. Regarding CBM family 28, the module from the endo-1,4-glucanase of Bacillus sp. 1139 binds to non-crystalline cellulose, cellooligosaccharides, and β-(1,3)(1,4)-glucans. For CBM Family 30, binding to cellulose has been demonstrated for the N-terminal module of Fibrobacter succinogenes CeIF. The C-terminal CBM44 module of the Clostridium thermocellum enzyme has been demonstrated to bind equally well cellulose and xyloglucan. CBM Family 72 includes modules of 130-180 residues found at the C-terminus glycoside hydrolases from various families, sometimes as tandem repeats. The CBM72 found on an endoglucanase from an uncultivated microorganism was found to bind a broad spectrum of polysaccharides including soluble and insoluble cellulose, beta-1,3/1,4-mixed linked glucans, xylan, and beta-mannan. CBM Family 79 includes modules of approx. 130 residues found so far only in ruminococcal proteins. Binding to various beta-glucans was shown for the R. flavefaciens GH9 enzyme.

Most preferred are CBM family 1 also referred to as “CBM1.”

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellulolytic enzyme or cellulase: The term “cellulolytic enzyme” or “cellulase” means one or more (e.g., several) enzymes that hydrolyze a cellulosic material. Such enzymes include endoglucanase(s) (e.g. EC 3.2.1.4), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman NW1 filter paper, microcrystalline cellulose, bacterial cellulose, algal cellulose, cotton, pretreated lignocellulose, etc. The most common total cellulolytic activity assay is the filter paper assay using Whatman NW1 filter paper as the substrate. The assay was established by the International Union of Pure and Applied Chemistry (IUPAC) (Ghose, 1987, Pure Appl. Chem. 59: 257-68).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Color clarification: During washing and wearing loose or broken fibers can accumulate on the surface of the fabrics. One consequence can be that the colors of the fabric appear less bright or less intense because of the surface contaminations. Removal of the loose or broken fibers from the textile will partly restore the original colors and looks of the textile. By the term “color clarification”, as used herein, is meant the partial restoration of the initial colors of textile.

Detergent component: the term “detergent component” is defined herein to mean the types of chemicals which can be used in detergent compositions. Examples of detergent components are surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers. The detergent composition may comprise of one or more of any type of detergent component.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Detergent composition: the term “detergent composition” refers to compositions that find use in the removal of undesired compounds from items to be cleaned, such as textiles, dishes, and hard surfaces. The detergent composition may be used to e.g. clean textiles, dishes and hard surfaces for both household cleaning and industrial cleaning and/or for fabric care. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, plastic, ceramic and metal counter tops and windows; carpet cleaners; oven cleaners; fabric fresheners; fabric softeners; and textile and laundry pre-spotters, as well as dish wash detergents). In addition to containing an enzyme of the invention, the detergent formulation may contain one or more additional enzymes (such as amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xyloglucanases, xylanases, pectin acetyl esterases, polygalacturonases, rhamnogalacturonases, other endo-beta-mannanases, exo-beta-mannanases (GH5 and/or GH26), licheninases, phosphodiesterases, pectin methylesterases, cellobiohydrolases, transglutaminases, nucleases, and combinations thereof, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti corrosion agents, enzyme inhibitors or stabilizers, enzyme activators, transferase(s), hydrolytic enzymes, oxido reductases, bluing agents and fluorescent dyes, antioxidants, and solubilizers.

Dish wash: The term “dish wash” refers to all forms of washing dishes, e.g. by hand dish wash (HDW) or automatic dish wash (ADW). Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

Dish washing composition: The term “dish washing composition” refers to compositions intended for cleaning dishes, table ware, pots, pans, cutlery and all forms of compositions for cleaning hard surfaces areas in kitchens. The present invention is not restricted to any particular type of dish wash composition or any particular detergent.

Endoglucanase: The term “endoglucanase” means an enzyme that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-beta-1,4 glucans such as cereal beta-D-glucans or xyloglucans, and other plant material containing cellulosic components. For purposes of the present invention, endoglucanase activity is determined using carboxymethyl cellulose (CMC) hydrolysis according to the procedure of Ghose, 1987, Pure and Appl. Chem. 59: 257-268. One unit of endoglucanase activity is defined as 1.0 μmole of reducing sugars produced per minute at 50° C., pH 4.8.

One particularly preferred class of endoglucanase are those of “family GH45,” which are classified as glycoside hydrolase Family 45 according to the terminology of Henrissat et al., Biochem. J. 280:309-316 (1991), as well as the Carbohydrate Active enZYmes database available at cazy.org. GH45 enzymes are endoglucanases of EC 3.2.1.4.

Expression: The term “expression” includes any step involved in the production of a variant including, but not limited to, transcription, post-transcriptional modification, translation, posttranslational modification, and secretion.

Expression vector: The term “expression vector” means a linear or circular DNA molecule that comprises a polynucleotide encoding a variant and is operably linked to control sequences that provide for its expression.

Fabric care: The term “fabric care,” also referred to as textile care, refers to treatments that retains or partly or fully restores the properties of the textile, e.g. by color clarification, antipilling or prevention of formation of pills on the textile surface.

Fragment: The term “fragment” means a polypeptide having one or more (e.g., several) amino acids absent from the amino and/or carboxyl terminus of a mature polypeptide; wherein the fragment retains the enzymatic activity of the full-length polypeptide.

Engineered: The term “engineered” means a synthetic construct.

Glycoside hydrolase: The term “glycoside hydrolase” (GH) means an enzyme that catalyzes the hydrolysis of a glycosidic bond between two or more carbohydrates or between a carbohydrate and a non-carbohydrate moiety. For more details, see, for example, Henrissat B., “A classification of glycosyl hydrolases based on amino-acid sequence similarities.” Biochem. J. 280:309-316 (1991), as well as the Carbohydrate Active enZYmes database available at cazy.org. Some glycoside hydrolases contain one or more catalytic domains and one or more carbohydrate binding modules (CBM), which are joined by one or more linker regions connecting these domains.

Exemplary glycoside hydrolase families with reported cellulase activities useful according to the present invention include those of families GH5, GH6, GH7, GH8, GH9, GH12, GH44, GH45, GH48, GH51, GH124, with family GH45 being particularly preferred.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Hybrid polypeptide: The term “hybrid polypeptide” means a polypeptide comprising domains from two or more polypeptides from different sources (origins), e.g., a binding module from one polypeptide and a catalytic domain from another polypeptide. The domains may be fused at the N-terminus or the C-terminus. Of particular interest herein are polypeptides comprising a binding module from one polypeptide (which may be naturally occurring or further modified), an engineered linker region, such as a proline-rich linker region, which is a synthetic construct, and a catalytic domain from another polypeptide (which may be naturally occurring or further modified).

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Improved property: The term “improved property” means a characteristic associated with a variant that is improved compared to the reference enzyme/parent enzyme. Some aspects of the invention relate to variants having an improvement factor above 1 when the variant is tested for a property of interest in a relevant assay, wherein the property of the reference enzyme/parent enzyme is given a value of 1.

Improved stability: The term “improved stability” means an enzyme having better stability in the presence of protease relative to the stability of a reference enzyme/parent enzyme, and includes, for example, proteolytic stability, in-detergent storage stability, improved stability during production of the detergent composition, as well as in-wash stability. The improvement in stability can be quantified by determining stability according to the assay described in Example 2 (linker stability assay—in the presence of protease), and/or Example 7 (In-wash Linker Stability Assay with proteases) herein.

Improved wash performance: The term “improved wash performance” is defined herein as an enzyme displaying an increased wash performance in a detergent composition relative to the wash performance of a reference enzyme/parent enzyme, e.g., by increased color clarification and/or anti-pilling effect, when evaluating the fresh samples and/or after the samples have been stored under the same conditions. The term “improved wash performance” includes wash performance in laundry but also in, e.g., hard surface cleaning such as automated dish wash (ADW).

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its mature form following N-terminal processing (e.g., removal of signal peptide).

Mature polypeptide coding sequence: The term “mature polypeptide coding sequence” means a polynucleotide that encodes a mature polypeptide having cellulase, such as endoglucanase activity.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent cellulase: The term “parent” or “parent cellulase” means any polypeptide with glycoside hydrolase activity, in particular cellulolytic or even endoglucanase activity, to which an alteration is made to produce the glycoside hydrolase variants comprising the carbohydrate binding module variants of the present invention.

Proline-rich linker: The term “proline-rich linker” means a sequence comprising one or more Pro-Pro, Pro-Xaa (or Xaa-Pro), Xaa-Pro-Xaa or Xaa-Xaa-Pro (or Pro-Xaa-Xaa) units, where Pro is the three-letter representation for the amino acid proline and Xaa is the three-letter representation for any amino acid. Preferably, the proline-rich linker comprises the above-noted units in repetition, e.g., PP, PPP, PPPP (SEQ ID NO: 27), PX, PXP, PS, PSP, PXPX (SEQ ID NO: 98), XP, XPX, SP, SPS, XPXP (SEQ ID NO: 99), XPXXPX (SEQ ID NO: 100), XXPXXP (SEQ ID NO: 101) and so forth, in combination and/or in succession.

In one aspect, the proline-rich linker comprises one or more of the following optionally repeating motifs: [P/S/T/R/K/D/E]P and P[S/T/R/K/D/E/N/Q]P[S/T/R/K/D/E] (SEQ ID NO: 102). In one aspect, the proline-rich linker comprises the following optionally repeating motifs: [S/T/R/K/D/E]P[S/T/R/K/D/E/N/Q], [P/S/T/R/K/D/E][P/S/T/R/K/D/E]P, and/or P[P/S/T/R/K/D/E][P/S/T/R/K/D/E]. In one aspect, the proline-rich linker comprises optionally repeating motifs of the same, or different, amino acids within the brackets as indicated: [P/S/T]P and P[S/E]PT (SEQ ID NO: 109).

In one aspect, the proline-rich linker comprises (a) (SP)a, a=2-10; (b) (PS)a, a=2-10; (c) Pb, b=4-20, preferably 4-15; (d) (PEPT (SEQ ID NO: 125))c, c=2-5; (e) (PSPT (SEQ ID NO: 104))d, d=2-5; (f) (P[S/T/R/K/D/E/N/Q]P[S/T/R/K/D/E] (SEQ ID NO: 102))e, e=2-5; (g) ([S/T/R/K/D/E]P)f, f=2-10, preferably 2-5; (h) ([S/T/R/K/D/E/N/Q]P[S/T/R/K/D/E])g, g=2-6; (i) ([S/T/R/K/D/E/N/Q][S/T/R/K/D/E/N/Q]P)h, h=2-5; (j) (TP)i, i=2-10; (k) ([S/T/P][S/T/P][S/T/P])j, j=2-11; (l) and/or combinations thereof, wherein combinations of the respective monomeric units are contemplated.

In one aspect, the proline-rich linker comprises a linker in Table A., below, such as PPPPPPP (SEQ ID NO: 31), PPPPPPPG (SEQ ID NO: 30), SPSPSPSPSP (SEQ ID NO: 58) or SPSPSPSPSPG (SEQ ID NO: 25).

Preferably, the proline-rich linker comprises at least 25% proline, e.g., at least 28% proline, at least 30% proline, at least 35% proline, at least 40% proline, at least 50% proline, such as at least 60%, at least 66%, at least 70%, at least 75%, at least 80%, at least 85%, at least 90% proline. Preferably, the proline-rich linker includes at least 4 amino acids and not more than 30 amino acids, such as 4-28 amino acids, preferably 4-20 amino acids, or even 4-10 amino acids, such as 4 amino acids, 5 amino acids, 6 amino acids, 7 amino acids, 8 amino acids, 9 amino acids or 10 amino acids.

Moreover, the linker region as defined herein can include an interface of an additional 1-2 amino acids at the connection to the catalytic domain and/or carbohydrate binding module.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well.

Variant: The term “variant” means a polypeptide comprising a substitution at one or more (e.g., several) positions and retaining the activity of the parent. A substitution means replacement of the amino acid occupying a position with a different amino acid. The carbohydrate binding module variants of the present invention have at least 20%, e.g., at least 40%, at least 50%, at least 60%, at least 70%, at least 80%, at least 90%, at least 95%, or at least 100% of the cellulose binding activity of the parent CBM, such as the polypeptide of SEQ ID NO: 173. The variants having glycoside hydrolase activity comprising the CBM variants have at least 20%, e.g., at least 40%, at least 50%, at least 60%, at least 70%, at least 80%, at least 90%, at least 95%, or at least 100% of the glycoside hydrolase activity of the parent, such as the polypeptide of SEQ ID NO: 1, SEQ ID NO: 2, SEQ ID NO: 3, SEQ ID NO: 4. A “variant” as used herein may also include a hybrid polypeptide.

Wash liquor: The term “wash liquor” refers to an aqueous solution containing a detergent composition in dilute form, such as but not limited to a detergent solution containing a laundry detergent composition in dilute form such as the wash liquor in a laundry process.

Whiteness: The term “Whiteness” is defined herein as a broad term with different meanings in different regions and for different consumers. Loss of whiteness can e.g. be due to greying, yellowing, or removal of optical brighteners/hueing agents. Greying and yellowing can be due to soil redeposition, body soils, coloring from e.g. iron and copper ions or dye transfer. Whiteness might include one or several issues from the list below: colorant or dye effects; incomplete stain removal (e.g. body soils, sebum etc.); redeposition (greying, yellowing or other discolorations of the object) (removed soils re-associate with other parts of textile, soiled or unsoiled); chemical changes in textile during application; and clarification or brightening of colors.

Wild-type: The term “wild-type” in reference to an amino acid sequence or nucleic acid sequence means that the amino acid sequence or nucleic acid sequence is a native or naturallyoccurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Variants

For purposes of the present invention, the carbohydrate binding module of SEQ ID NO: 173 is used to determine the corresponding amino acid residue in another carbohydrate binding module. The amino acid sequence of another carbohydrate binding module is aligned with the polypeptide disclosed in SEQ ID NO: 173, and based on the alignment, the amino acid position number corresponding to any amino acid residue in the polypeptide disclosed in SEQ ID NO: 173 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another carbohydrate binding module can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other carbohydrate binding module has diverged from the polypeptide of SEQ ID NO: 173 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+” or “,”), e.g., “Gly205Arg+Ser411Phe” or “G205R+S411F” or “Gly205Arg, Ser411 Phe” or “G205R, S411F”, representing substitutions at positions 205 and 411 of glycine (G) with arginine I and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+” or “,”), e.g., “Gly195*+Ser411*” or “G195*+S411*” or “Gly195*, Ser411*” or “G195*, S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+” or “,”), e.g., “Arg170Tyr+Gly195Glu” or “R170Y+G195E” or “Arg170Tyr,Gly195Glu” or “R170Y,G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### NOMENCLATURE

For purposes of the present invention, brackets are used to indicate alternative amino acids (using their one letter codes) at a particular position in a sequence. For example, the nomenclature [S/E] means that the amino acid at this position may be a serine (Ser, S) or a glutamic acid (Glu, E). Likewise, the nomenclature [P/S/T] means that the amino acid at this position may be a proline (Pro, P), a serine (Ser, S), or a threonine (Thr, T), and so forth for other combinations as described herein. Amino acids indicated within brackets using this nomenclature may be separated by a vertical line or in some instances no line e.g. [P/S/T] can also be designated as [PST].

In some instance, a sequence motif includes more than one set of brackets, each of which independently represents a position in a sequence. Thus, P[S/T/R/K/D/E/N/Q]P[S/T/R/K/D/E](SEQ ID NO: 102) means that P, conservative amino acid, is in the first position; any of S, T, R, K, D, E, N, or Q are in the second position; P, conservative amino acid, is in the third position; and any of S, T, R, K, D, or E are in the fourth position. The motif represented by this designation may then be any of PSPS (SEQ ID NO: 103), PSPT (SEQ ID NO: 104), PSPR (SEQ ID NO: 105), PSPK (SEQ ID NO: 106), PSPD (SEQ ID NO: 107), PSPE (SEQ ID NO: 108) and so on.

Unless otherwise limited further, the amino acid X (or Xaa) is used herein to represent any of the 20 natural amino acids.

### DETAILED DESCRIPTION OF THE INVENTION

Many proteins are comprised of structured domains connected by linkers. For example, cellulases and other glycoside hydrolases (GH) are often found as modular enzymes having one or more catalytic domains, which may be connected to one or more CBMs via a peptide known as a linker, which is sometimes partially glycosylated. The catalytic domain is responsible for the hydrolytic degradation of cellulose, while the CBM, when present, works by increasing the effective concentration of enzyme near the substrate surface. In contrast, linkers are generally flexible connectors that provide connectivity between structured domains, but their functional role is largely unknown.

Cellulases in particular, are often cleaved (nicked) in exposed regions or partially or fully degraded by proteases in liquid detergents. Most commonly, the protease cleaves in the unstructured linker region of the cellulase and, for example in detergent applications, thereby reduces the ability of the cellulase to remove fuzz and pill and maintaining or restoring the colors of the textile by reducing its ability to bind to the insoluble cellulose substrate. The loss in binding affinity strongly impacts the performance of cellulases which is why a protease stable molecule is highly valuable in the liquid laundry/dish detergent segment, as well as in softeners.

Similarly, proteolytic cleavage of peptide binds in the CBM module can result in loss of the ability of the cellulase to bind to the insoluble cellulose substrate and thereby impact the performance.

The present invention relates to a variant of a carbohydrate binding module having cellulose binding activity. The present invention also relates to glycoside hydrolase enzymes comprising the carbohydrate binding module variants of the invention, in particular to variants of a glycoside hydrolase having a three-domain structure with one or more catalytic domains connected to one or more carbohydrate binding modules via a linker. Surprisingly, the present inventors have found that carbohydrate binding module variants can provide improved stability of the resultant glycoside hydrolase enzyme comprising the carbohydrate binding module variant, and make the glycoside hydrolase enzyme more stable, i.e., less susceptible to proteolytic cleavage.

In some embodiments, the glycoside hydrolase variants comprising the carbohydrate binding module variants also include peptide stretches that make the native linker more stable, i.e., less susceptible to proteolytic cleavage. Preferably, the glycoside hydrolase variants comprise a linker that is an engineered linker region, such as a proline-rich linker as set forth herein, in addition to the carbohydrate binding module variant.

However, before the present work, it was not known that modifications in the carbohydrate binding module of a glycoside hydrolase could make the resultant glycoside hydrolase more stable, i.e. less susceptible to proteolytic cleavage.

Family 1 CBMs include modules of approximately 40 residues found almost exclusively in fungi. The cellulose-binding function has been demonstrated in many cases, and appears to be mediated by three aromatic residues separated by about 10.4 angstrom and which form a flat surface. Family 1 CBMs also have a conserved C-terminal SQCL (SEQ ID NO: 201), which is believed to be important for binding affinity. Surprisingly, the present inventors have found that modifications in the carbohydrate binding module, including modifications corresponding to the Ser and/or Leu of the C-terminal SQCL (SEQ ID NO: 201) provide improved stability without sacrificing cellulose binding activity.

The present inventors have also surprisingly found that modifications in the carbohydrate binding module, including modifications corresponding to the Ser and/or Leu of the C-terminal SQCL (SEQ ID NO: 201) provide improved stability to a glycoside hydrolase enzyme comprising the carbohydrate binding module variant, without sacrificing glycoside hydrolase activity of the polypeptide.

### Carbohydrate Binding Module Variants

The invention relates to a variant of a carbohydrate binding module comprising a substitution at one or more positions corresponding to positions 14, 18, 21, 22, 25, 27, 28, 29, 34, 37 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity.

Exemplary starting CBMs useful for modifying to provide variants according to the invention are those provided in any of SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, or SEQ ID NO: 200. Thus, in one embodiment, the variant of a carbohydrate binding module has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, sequence identity to the polypeptide of SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, or SEQ ID NO: 200.

In one aspect, the number of alterations in the variants of the present invention is 1-20, e.g., 1-10 and 1-5, such as 1, 2, 3, 4, 5, 6, 7, 8, 9 or 10 alterations.

In another aspect, a variant comprises a substitution at one or more positions corresponding to positions 14, 18, 21, 22, 25, 27, 28, 29, 34, and 37 of SEQ ID NO: 173. In another aspect, a variant comprises a substitution at two positions corresponding to any of positions 14, 18, 21, 22, 25, 27, 28, 29, 34, and 37. In another aspect, a variant comprises a substitution at three positions corresponding to any of positions 14, 18, 21, 22, 25, 27, 28, 29, 34, and 37. In another aspect, a variant comprises a substitution at four positions corresponding to positions 14, 18, 21, 22, 25, 27, 28, 29, 34, and 37. In another aspect, a variant comprises a substitution at five positions corresponding to positions 14, 18, 21, 22, 25, 27, 28, 29, 34, and 37. In another aspect, a variant comprises a substitution at six positions corresponding to positions 14, 18, 21, 22, 25, 27, 28, 29, 34, and 37. In another aspect, a variant comprises a substitution at seven positions corresponding to positions 14, 18, 21, 22, 25, 27, 28, 29, 34, and 37. In another aspect, a variant comprises a substitution at eight positions corresponding to positions 14, 18, 21, 22, 25, 27, 28, 29, 34, and 37. In another aspect, a variant comprises a substitution at nine positions corresponding to positions 14, 18, 21, 22, 25, 27, 28, 29, 34, and 37. In another aspect, a variant comprises a substitution at ten positions corresponding to positions 14, 18, 21, 22, 25, 27, 28, 29, 34, and 37. In another aspect, a variant comprises a substitution at each position corresponding to position 14, 18, 21, 22, 25, 27, 28, 29, 34, and 37.

The invention also relates to a variant of a carbohydrate binding module comprising a substitution at one or more positions, preferably both positions, corresponding to positions 34 and 37 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity.

The invention also relates to a variant of a carbohydrate binding module comprising a substitution at position corresponding to position 14 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 14 is substituted with Ala, Arg, Asn, Asp, Cys, Gln, Glu, Gly, His, lie, Leu, Lys, Met, Phe, Pro, Ser, Thr, Trp, Tyr, or Val, preferably with Trp. In another aspect, the variant comprises or consists of the substitution Y14W of the polypeptide of SEQ ID NO: 173.

The invention also relates to a variant of a carbohydrate binding module comprising a substitution at position corresponding to position 18 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 18 is substituted with Ala, Arg, Asn, Asp, Cys, Gln, Glu, Gly, His, lie, Leu, Lys, Met, Phe, Pro, Ser, Thr, Trp, Tyr, or Val, preferably with Lys or Arg. In another aspect, the variant comprises or consists of the substitution T18K or T18R of the polypeptide of SEQ ID NO: 173.

The invention also relates to a variant of a carbohydrate binding module comprising a substitution at position corresponding to position 21 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 21 is substituted with Ala, Arg, Asn, Asp, Cys, Gln, Glu, Gly, His, lie, Leu, Lys, Met, Phe, Pro, Ser, Thr, Trp, Tyr, or Val, preferably with Glu. In another aspect, the variant comprises or consists of the substitution V21E of the polypeptide of SEQ ID NO: 173.

The invention also relates to a variant of a carbohydrate binding module comprising a substitution at position corresponding to position 22 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 22 is substituted with Ala, Arg, Asn, Asp, Cys, Gln, Glu, Gly, His, lie, Leu, Lys, Met, Phe, Pro, Ser, Thr, Trp, Tyr, or Val, preferably with Glu, Asn, Pro. In another aspect, the variant comprises or consists of the substitution A22E or A22N or A22P of the polypeptide of SEQ ID NO: 173.

The invention also relates to a variant of a carbohydrate binding module comprising a substitution at position corresponding to position 25 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 25 is substituted with Ala, Arg, Asn, Asp, Cys, Gln, Glu, Gly, His, lie, Leu, Lys, Met, Phe, Pro, Ser, Thr, Trp, Tyr, or Val, preferably with Glu. In another aspect, the variant comprises or consists of the substitution T25E of the polypeptide of SEQ ID NO: 173.

The invention also relates to a variant of a carbohydrate binding module comprising a substitution at position corresponding to position 27 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 27 is substituted with Ala, Arg, Asn, Asp, Cys, Gln, Glu, Gly, His, lie, Leu, Lys, Met, Phe, Pro, Ser, Thr, Trp, Tyr, or Val, preferably with Ile. In another aspect, the variant comprises or consists of the substitution T27I of the polypeptide of SEQ ID NO: 173.

The invention also relates to a variant of a carbohydrate binding module comprising a substitution at position corresponding to position 28 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 28 is substituted with Ala, Arg, Asn, Asp, Cys, Gln, Glu, Gly, His, lie, Leu, Lys, Met, Phe, Pro, Ser, Thr, Trp, Tyr, or Val, preferably with Pro. In another aspect, the variant comprises or consists of the substitution Q28P of the polypeptide of SEQ ID NO: 173.

The invention also relates to a variant of a carbohydrate binding module comprising a substitution at positions corresponding to position 29 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 29 is substituted with Ala, Arg, Asn, Asp, Cys, Gln, Glu, Gly, His, lie, Leu, Lys, Met, Phe, Pro, Ser, Thr, Trp, Tyr, or Val, preferably with Pro. In another aspect, the variant comprises or consists of the substitution L29P of the polypeptide of SEQ ID NO: 173.

The invention also relates to a variant of a carbohydrate binding module comprising a substitution at position corresponding to position 34 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 34 is substituted with Ala, Arg, Asn, Asp, Cys, Gln, Glu, Gly, His, lie, Leu, Lys, Met, Phe, Pro, Ser, Thr, Trp, Tyr, or Val, preferably with His, Tyr, Lys, Arg. In another aspect, the variant comprises or consists of the substitution S34H, S34Y, S34K, S34R of the polypeptide of SEQ ID NO: 173.

The invention also relates to a variant of a carbohydrate binding module comprising a substitution at positions corresponding to position 37 of the polypeptide of SEQ ID NO: 173. The variant of the CBM comprises an amino acid sequence that is from about 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173, and has cellulose binding activity. In an embodiment, In another aspect, the amino acid at a position corresponding to position 37 is substituted with Ala, Arg, Asn, Asp, Cys, Gln, Glu, Gly, His, lie, Leu, Lys, Met, Phe, Pro, Ser, Thr, Trp, Tyr, or Val, preferably with Arg. In another aspect, the variant comprises or consists of the substitution L37R of the polypeptide of SEQ ID NO: 173.

In another aspect, the variant comprises or consists of one or more substitutions selected from the group consisting of Y14W, T18K, T18R, V21E, A22E, A22N, A22P, T25E, T27I, Q28P, L29P, S34H, S34Y, S34K, S34R, and L37R of the polypeptide of SEQ ID NO: 173.

Preferred CBM variants have substitutions selected from the group consisting of:

S34H,

S34Y,

S34K,

S34R,

Y14W,

T18K,

L37R,

S34Y+L37R,

T18K+S34H,

S34H+L37R,

T18K+S34Y+L37R,

T18R,

V21E,

A22E,

A22P,

A22N,

T25E,

T27I,

Q28P,

L29P,

S34H+L37R+Y14W,

S34H+L37R+T18K,

S34H+L37R+Y14W+T18K,

S34H+L37R+T18R,

S34H+L37R+V21E,

S34H+L37R+A22E,

S34H+L37R+A22P,

S34H+L37R+A22N,

S34H+L37R+Q28P,

S34H+L37R+L29P,

S34H+L37R+T25E,

S34H+L37R+T27I,

S34H+L37R+V21E+A22E,

S34H+L37R+V21E+A22N,

Y14W+T18R+S34H+L37R,

Y14W+V21E+S34H+L37R,

Y14W+A22E+S34H+L37R,

Y14W+A22P+S34H+L37R,

Y14W+A22N+S34H+L37R,

Y14W+Q18P+S34H+L37R,

Y14W+L29P+S34H+L37R,

Y14W+T25E+S34H+L37R,

Y14W+T27I+S34H+L37R,

Y14W+V21E+A22E+S34H+L37R,

Y14W+V21E+A22N+S34H+L37R,

T18K+V21E+S34H+L37R,

T18K+A22E+S34H+L37R,

T18K+A22P+S34H+L37R,

T18K+A22N+S34H+L37R,

T18K+Q28P+S34H+L37R,

T18K+L29P+S34H+L37R,

T18K+T25E+S34H+L37R,

T18K+T27I+S34H+L37R,

T18K+V21E+A22E+S34H+L37R,

T18K+V21E+A22N+S34H+L37R,

Y14W+T18K+V21E+S34H+L37R,

Y14W+T18K+A22E+S34H+L37R,

Y14W+T18K+A22P+S34H+L37R,

Y14W+T18K+A22N+S34H+L37R,

Y14W+T18K+Q28P+S34H+L37R,

Y14W+T18K+L29P+S34H+L37R,

Y14W+T18K+T25E+S34H+L37R,

Y14W+T18K+T27I+S34H+L37R,

Y14W+T18K+V21E+A22E+S34H+L37R,

Y14W+T18K+A21E+A22N+S34H+L37R,

of SEQ ID NO: 173.

Polypeptides having Glycoside Hydrolase Activity Comprising Carbohydrate Binding Module Variants

The present invention also relates to variant polypeptides having glycoside hydrolase activity comprising one or more catalytic domains from a polypeptide having glycoside hydrolase activity and a carbohydrate binding module variant, as described above, which are joined by one or more linkers. The variant polypeptide having glycoside hydrolase activity exhibits improved linker stability and/or improved CBM stability in comparison with the parent in an aqueous composition comprising a protease.

As noted above, glycoside hydrolases contain one or more catalytic domains and one or more carbohydrate binding modules (CBM), which are joined by one or more linker regions connecting these domains.

Particularly preferred enzymes are those having cellulase, such as endoglucanase activity. In particular, relevant catalytic domains are from enzymes of the glycoside hydrolase family 45 (GH45), using the nomenclature of Henrissat et al. outlined on the CAZY database available at cazy.org

The catalytic domain can comprise a wild type or variant thereof.

In an embodiment, the catalytic domain comprises an amino acid sequence having at least 70% sequence identity, e.g., at least 75% sequence identity, at least 80% sequence identity, at least 85% sequence identity, at least 90% sequence identity, at least 91%, at least 92%, at least 93%, at least 94%, at least 95% sequence identity, at least 96% sequence identity, at least 97% sequence identity, at least 98% sequence identity, at least 99% sequence identity to the amino acid sequence as shown in positions 1-212 of SEQ ID NO: 1.

In an embodiment, the catalytic domain comprises an amino acid sequence having at least 70% sequence identity, e.g., at least 75% sequence identity, at least 80% sequence identity, at least 85% sequence identity, at least 90% sequence identity, at least 91%, at least 92%, at least 93%, at least 94%, at least 95% sequence identity, at least 96% sequence identity, at least 97% sequence identity, at least 98% sequence identity, at least 99% sequence identity to the amino acid sequence as shown in positions 1-211 of SEQ ID NO: 2.

In an embodiment, the catalytic domain comprises an amino acid sequence having at least 70% sequence identity, e.g., at least 75% sequence identity, at least 80% sequence identity, at least 85% sequence identity, at least 90% sequence identity, at least 91%, at least 92%, at least 93%, at least 94%, at least 95% sequence identity, at least 96% sequence identity, at least 97% sequence identity, at least 98% sequence identity, at least 99% sequence identity to the amino acid sequence as shown in positions 1-210 of SEQ ID NO: 3.

In an embodiment, the catalytic domain comprises an amino acid sequence having at least 70% sequence identity, e.g., at least 75% sequence identity, at least 80% sequence identity, at least 85% sequence identity, at least 90% sequence identity, at least 91%, at least 92%, at least 93%, at least 94%, at least 95% sequence identity, at least 96% sequence identity, at least 97% sequence identity, at least 98% sequence identity, at least 99% sequence identity to the amino acid sequence as shown in positions 1-210 of SEQ ID NO: 4.

In one aspect, the catalytic domain further comprises a number of substitutions in the variants of the present invention is 2-20, e.g., 2-10 and 2-5, such as 2, 3, 4, 5, 6, 7, 8, 9 or 10 substitutions.

In another aspect, the variant comprises or consists of two substitution at positions selected among the positions corresponding to: 25, 32, 41, 44, 56, 77, 104, 132, 134, 146, 147, 156, 162, 169, 183, 186, 194, or 201 in SEQ ID NO: 1. In another aspect, the amino acid at this position is substituted with Ala, Arg, Asn, Asp, Cys, Gln, Glu, Gly, His, lie, Leu, Lys, Met, Phe, Pro, Ser, Thr, Trp, Tyr, or Val.

A preferred catalytic domain comprises SEQ ID NO: 5 having mutations A32S S56A N134D A146D Q147R Q169Y F183V.

The polypeptides having glycoside hydrolase activity according to the invention can comprise a native linker.

In some embodiments, the variant polypeptides having glycoside hydrolase activity according to the invention comprise a proline-rich amino acid sequence connecting the one or more catalytic cores with the CBM1 variants.

The proline-rich linkers as described herein comprise one or more Pro-Pro, Pro-Xaa (or Xaa-Pro), Xaa-Pro-Xaa or Xaa-Xaa-Pro (or Pro-Xaa-Xaa) units, e.g., PPPP (SEQ ID NO: 27), PXPX (SEQ ID NO: 98), XPXP (SEQ ID NO: 99), XPXXPX (SEQ ID NO: 100), XXPXXP (SEQ ID NO: 101) and so forth, optionally further repeating.

For example, the linker region may comprise at least 25% proline, e.g. at least 28% proline, at least 30% proline, at least 40% proline, at least 50% proline, such as at least 60%, at least 66%, at least 70%, at least 75%, at least 80%, at least 85%, at least 90% proline. In other embodiments, the linker comprises at least 50% proline, such as at least 60%, at least 66%, at least 70%, at least 75%, at least 80%, at least 85%, at least 90% and has an overall negative charge. For example, the linker region comprises amino acids of an acidic nature.

Preferred linker regions have a length of at least 4 amino acids and not more than 30 amino acids, such as 4-28 amino acids, preferably 4-20 amino acids, or even 4-10 amino acids, such as 4 amino acids, 5 amino acids, 6 amino acids, 7 amino acids, 8 amino acids, 9 amino acids or 10 amino acids.

Exemplary linker regions comprise one or more of the following optionally repeating motifs:

(SEQ ID NO: 102)
[P/S/T/R/K/D/E]P
and
P[S/T/R/K/D/E/N/Q]P[S/T/R/K/D/E]

Other preferred linker regions include the following optionally repeating motifs:

[S/T/R/K/D/E]P[S/T/R/K/D/E/N/Q]
[P/S/T/R/K/D/E][P/S/T/R/K/D/E]P,
and/or
P[P/S/T/R/K/D/E][P/S/T/R/K/D/E].

Particularly preferred linkers include the optionally repeating motifs of the same, or different, amino acids within the brackets as indicated:

(SEQ ID NO: 109)
[P/S/T]P
and
P[S/E]PT

Or more particularly, the optionally repeating motif represented by [P/S/T]P includes PPPPPP (SEQ ID NO: 29), as well as PPSPTP (SEQ ID NO: 110), PPTPTP (SEQ ID NO: 111), PPSPSP (SEQ ID NO: 112), SPPPTP (SEQ ID NO: 113), SPTPPP (SEQ ID NO: 114), SPPPPP (SEQ ID NO: 115), SPTPTP (SEQ ID NO: 116), TPPPSP (SEQ ID NO: 117), TPSPPP (SEQ ID NO: 118), TPPPPP (SEQ ID NO: 119), TPSPSP (SEQ ID NO: 120), and the optionally repeating motif represented by P[S/E]PT (SEQ ID NO: 109) would include PSPTPEPT (SEQ ID NO: 121), PSPTPEPTPSPTPEPT (SEQ ID NO: 122), PEPTPSPT (SEQ ID NO: 123), PEPTPSPTPSPT (SEQ ID NO: 124) and so forth.

Exemplary linkers further comprise
(a) (SP)ₐ, a=2-10;
(b) (PS)ₐ, a=2-10;
(c) P_{b}, b=4-20, preferably 4-15;
(d) (PEPT (SEQ ID NO: 125))_{c}, c=2-5;
(e) (PSPT (SEQ ID NO: 104))_{d}, d=2-5;
(f) (P[S/T/R/K/D/E/N/Q]P[S/T/R/K/D/E] (SEQ ID NO: 102))ₑ, e=2-5;
(g) ([S/T/R/K/D/E]P)_{f}, f=2-10, preferably 2-5;
(h) ([S/T/R/K/D/E/N/Q]P[S/T/R/K/D/E])_{g}, g=2-6;
(i) ([S/T/R/K/D/E/N/Q][S/T/R/K/D/E/N/Q]P)ₕ, h=2-5;
(j) (TP)ᵢ, i=2-10;
(k) ([S/T/P][S/T/P][S/T/P])ⱼ, j=2-11;
(l) and/or combinations thereof, wherein combinations of the respective monomeric units are contemplated.

When combinations of these motifs are included, the minimal repeating unit is a monomeric unit. For example, linkers including SPPEPT (SEQ ID NO: 126), SPPSPT (SEQ ID NO: 127), PSPEPT (SEQ ID NO: 128), PSPSPT (SEQ ID NO: 129).

Additional exemplary linkers include:

(SEQ ID NO: 130)
SPSP,
(SEQ ID NO: 131)
SPSPSP,
(SEQ ID NO: 132)
SPSPSPSP,
(SEQ ID NO: 58)
SPSPSPSPSP,
(SEQ ID NO: 133)
SPSPSPSPSPSP,
(SEQ ID NO: 134)
SPSPSPSPSPSPSP,
(SEQ ID NO: 135)
SPSPSPSPSPSPSPSP,
(SEQ ID NO: 27)
PPPP,
(SEQ ID NO: 28)
PPPPP,
(SEQ ID NO: 29)
PPPPPP,
(SEQ ID NO: 31)
PPPPPPP,
(SEQ ID NO: 136)
PPPPPPPP,
(SEQS ID NO: 137)
PPPPPPPPP,
(SEQ ID NO: 138)
PPPPPPPPPP,
(SEQ ID NO: 139)
PPPPPPPPPPP,
(SEQ ID NO: 140)
PPPPPPPPPPPP,
(SEQ ID NO: 141)
PPPPPPPPPPPPP,
(SEQ ID NO: 142)
PPPPPPPPPPPPPP,
(SEQ ID NO: 143)
PPPPPPPPPPPPPPP,
(SEQ ID NO: 144)
PEPTPEPT,
(SEQ ID NO: 145)
PEPTPEPTPEPT,
(SEQ ID NO: 146)
PEPTPEPTPEPTPEPT,
(SEQ ID NO: 79)
PEPTPEPTPEPTPEPTPEPT,
(SEQ ID NO: 147)
PSPTPSPT,
(SEQ ID NO: 148)
PSPTPSPTPSPT,
(SEQ ID NO: 149)
PSPTPSPTPSPTPSPT,
SEQ ID NO: 150)
PSPTPSPTPSPTPSPTPSPT,
(SEQ ID NO: 151)
SPSSPS,
(SEQ ID NO: 152)
SPSSPSSPS,
(SEQ ID NO: 153)
SPSSPSSPSSPS,
(SEQ ID NO: 154)
SPSSPSSPSSPSSPS,
(SEQ ID NO: 155)
TPTTPT,
(SEQ ID NO: 156)
TPTTPTTPT,
(SEQ ID NO: 157)
TPTTPTTPTTPT,
(SEQ ID NO: 158)
TPTTPTTPTTPTTPT,
(SEQ ID NO: 159)
PEPTPRPTPEPTPRPT,
(SEQ ID NO: 160)
PEPTPKPTPEPTPKPT,
(SEQ ID NO: 161)
PEPTPQPTPEPTPQPT,
(SEQ ID NO: 162)
PRPTPEPTPRPT,
(SEQ ID NO: 163)
PKPTPEPTPKPT,
(SEQ ID NO: 164)
PEPTPQPT,
(SEQ ID NO: 165)
PEPTPQPTPEPT,
(SEQ ID NO: 85)
PEPTPRPTPEPTPRPTG,
(SEQ ID NO: 87)
PEPTPKPTPEPTPKPTG,
(SEQ ID NO: 88)
PEPTPQPTPEPTPQPTG,
(SEQ ID NO: 89)
PRPTPEPTPRPTG,
(SEQ ID NO: 90)
PKPTPEPTPKPTG,
(SEQ ID NO: 91)
PEPTPQPTG,
(SEQ ID NO: 92)
PEPTPQPTPEPTG,
(SEQ ID NO: 82)
PPPGGPGGPGTPTSTAPGSGPTSPGGGSG,
(SEQ ID NO: 166)
TTPPTPTPTPTP;
(SEQ ID NO: 167)
TTPTPPTPTPTPTP,
(SEQ ID NO: 168)
TTPTPTPPTPTPTPTP,
(SEQ ID NO: 169)
TPPTPPTPPTPPTPPTPPTPPTPPTPPTPPTPP.

Additional exemplary linkers comprise the above-mentioned linkers, as well as a C-terminal glycine, for example, SPSPG (SEQ ID NO: 24), SPSPSPG, SPSPSPSPG SPSPSPSPSPG (SEQ ID NO: 25), SPSPSPSPSPSPG, SPSPSPSPSPSPSPG, SPSPSPSPSPSPSPSPG, PPPPG, PPPPPG, PPPPPPG, PPPPPPPG (SEQ ID NO: 30), PPPPPPPPG (SEQ ID NO: 32), PPPPPPPPPG (SEQ ID NO: 33), PPPPPPPPPPG (SEQ ID NO: 34), PPPPPPPPPPPG (SEQ ID NO: 35), PPPPPPPPPPPPG (SEQ ID NO: 170), PPPPPPPPPPPPPG (SEQ ID NO: 36), PPPPPPPPPPPPPPG (SEQ ID NO: 171), PPPPPPPPPPPPPPPG (SEQ ID NO: 172), PEPTPEPTG (SEQ ID NO: 37), PEPTPEPTPEPTG (SEQ ID NO: 38), PEPTPEPTPEPTPEPTG (SEQ ID NO: 39), PEPTPEPTPEPTPEPTPEPTG (SEQ ID NO: 40), PSPTPSPTG, PSPTPSPTPSPTG, PSPTPSPTPSPTPSPTG (SEQ ID NO: 41), PSPTPSPTPSPTPSPTPSPTG (SEQ ID NO: 42), SPSSPSG (SEQ ID NO: 94), SPSSPSSPSG (SEQ ID NO: 95), SPSSPSSPSSPSG (SEQ ID NO: 19), SPSSPSSPSSPSSPSG (SEQ ID NO: 20), TPTTPTG (SEQ ID NO: 96), TPTTPTTPTG (SEQ ID NO: 97), TPTTPTTPTTPTG (SEQ ID NO: 17), TPTTPTTPTTPTTPTG, PEPTPRPTPEPTPRPTG (SEQ ID NO: 85), PEPTPKPTPEPTPKPTG (SEQ ID NO: 87), PEPTPQPTPEPTPQPTG (SEQ ID NO: 88), PRPTPEPTPRPTG (SEQ ID NO: 89), PKPTPEPTPKPTG (SEQ ID NO: 90), PEPTPQPTG (SEQ ID NO: 91), PEPTPQPTPEPTG (SEQ ID NO: 92), PPPGGPGGPGTPTSTAPGSGPTSPGGGSG (SEQ ID NO: 82), TTPPTPTPTPTPG (SEQ ID NO: 12); TTPTPPTPTPTPTPG (SEQ ID NO: 13), TTPTPTPPTPTPTPTPG (SEQ ID NO: 14), TTPTPTPTPPTPTPTPTPG (SEQ ID NO: 15), TPPTPPTPPTPPTPPTPPTPPTPPTPPTPPTPPG (SEQ ID NO: 16).

Particularly preferred linkers are those comprising primarily or exclusively proline, e.g., PPPP (SEQ ID NO: 27), PPPPP (SEQ ID NO: 28), PPPPPP (SEQ ID NO: 29), PPPPPPP (SEQ ID NO: 31), PPPPPPPP (SEQ ID NO: 136), PPPPPPPPP (SEQ ID NO: 137), PPPPPPPPPP (SEQ ID NO: 138), PPPPPPPPPPP (SEQ ID NO: 139), PPPPPPPPPPPP (SEQ ID NO: 140), PPPPPPPPPPPPP (SEQ ID NO: 141), PPPPPPPPPPPPPP (SEQ ID NO: 142), PPPPPPPPPPPPPPP (SEQ ID NO: 143), PPPPG, PPPPPG, PPPPPPG, PPPPPPPG (SEQ ID NO: 30), PPPPPPPPG (SEQ ID NO: 32), PPPPPPPPPG (SEQ ID NO: 33), PPPPPPPPPPG (SEQ ID NO: 34), PPPPPPPPPPPG (SEQ ID NO: 35), PPPPPPPPPPPPG (SEQ ID NO: 170), PPPPPPPPPPPPPG (SEQ ID NO: 36), PPPPPPPPPPPPPPG (SEQ ID NO: 171), PPPPPPPPPPPPPPPG (SEQ ID NO: 172).

For the above-contemplated embodiments, one of skill in the art will appreciate that the objective is to replace the linker of the parent of interest, with the proline-rich linkers herein to provide additional stability.

In an alternative embodiment, the linker can be considered as a variant of the linker of the parent molecule, having stabilizing point mutations, including mutations to proline.

In a particularly preferred embodiment, the linker is selected from any of the following in Table A:

TABLE A
Preferred Linkers

TTPPTPTPTPTPG (SEQ ID NO: 12)
TTPTPPTPTPTPTPG (SEQ ID NO: 13)
TTPTPTPPTPTPTPTPG (SEQ ID NO: 14)
TTPTPTPTPPTPTPTPTPG (SEQ ID NO: 15)
TPPTPPTPPTPPTPPTPPTPPTPPTPPTPPTPPG (SEQ ID NO: 16)
TPTTPTTPTTPTG (SEQ ID NO: 17)
TPTTPTTPTTPTTPTTPTG (SEQ ID NO: 18)
SPSSPSSPSSPSG (SEQ ID NO: 19)
SPSSPSSPSSPSSPSG (SEQ ID NO: 20)
SPPSPPSPPSPPSPPG (SEQ ID NO: 21)
SPPSPPSPPSPPSPPSPPSPPSPPSPPSPPG (SEQ ID NO: 22)
PPSSPSSPSSPSSPSSPSSPSG (SEQ ID NO: 23)
SPSPG (SEQ ID NO: 24)
SPSPSPSPSPG (SEQ ID NO: 25)
TPTPTPTPTPG (SEQ ID NO: 26)
PPPP (SEQ ID NO: 27)
PPPPP (SEQ ID NO: 28)
PPPPPP (SEQ ID NO: 29)
PPPPPPPG (SEQ ID NO: 30)
PPPPPPP (SEQ ID NO: 31)
PPPPPPPPG (SEQ ID NO: 32)
PPPPPPPPPG (SEQ ID NO: 33)
PPPPPPPPPPG (SEQ ID NO: 34)
PPPPPPPPPPPG (SEQ ID NO: 35)
PPPPPPPPPPPPPG (SEQ ID NO: 36)
PEPTPEPTG (SEQ ID NO: 37)
PEPTPEPTPEPTG (SEQ ID NO: 38)
PEPTPEPTPEPTPEPTG (SEQ ID NO: 39)
PEPTPEPTPEPTPEPTPEPTG (SEQ ID NO: 40)
PSPTPSPTPSPTPSPTG (SEQ ID NO: 41)
PSPTPSPTPSPTPSPTPSPTG (SEQ ID NO: 42)
PQPTPQPTG (SEQ ID NO: 43)
PDPTPDPTG (SEQ ID NO: 44)
PRPTPEPTG (SEQ ID NO: 45)
PQPTPEPTG (SEQ ID NO: 46)
PSPNSPNSPNG (SEQ ID NO: 47)
PEPTPRPTG (SEQ ID NO: 48)
PQPTPEPTPQPTPEPTPQPTPEPTPQPTG (SEQ ID NO: 49)
PDPTPDPTPDPTG (SEQ ID NO: 50)
PQPTPQPTPQPTPQPTG (SEQ ID NO: 51)
PQPTPEPTPQPTPEPTG (SEQ ID NO: 52)
SPSPSPSPPPG (SEQ ID NO: 53)
SPSPSPSPDPG (SEQ ID NO: 54)
SPSPSPSPKPG (SEQ ID NO: 55)
SPSPSPSPAPG (SEQ ID NO: 56)
SPSPSPSPSPSG (SEQ ID NO: 57)
SPSPSPSPSP (SEQ ID NO: 58)
SPSPSPSPSPS (SEQ ID NO: 59)
SPSPSPSPSPP (SEQ ID NO: 60)
SPSPSPSPSPE (SEQ ID NO: 61)
SPSPSPSPSPN (SEQ ID NO: 62)
SPSPSPSPSPGG (SEQ ID NO: 63)
SPSPSPSPSPK (SEQ ID NO: 64)
PEPTPEPTP (SEQ ID NO: 65)
PEPTPEPTR (SEQ ID NO: 66)
PEPTPEPTPEPTP (SEQ ID NO: 67)
PEPTPEPTPEPTPEPTPSPTG (SEQ ID NO: 68)
PEPTPEPTPEPTPEPTPTPTG (SEQ ID NO: 69)
PEPTPEPTPEPTPEPTPGPTG (SEQ ID NO: 70)
PEPTPEPTPEPTPEPTPDPTG (SEQ ID NO: 71)
PEPTPEPTPEPTPEPTPETG (SEQ ID NO: 72)
PEPTPEPTPEPTPEPTPEPTD (SEQ ID NO: 73)
PEPTPEPTE (SEQ ID NO: 74)
PEPTPEPTPEPTPEPTPEP (SEQ ID NO: 75)
PEPTPEPTPEPTPEPTPSPT (SEQ ID NO: 76)
PEPTPEPTPEPTPEPTPRPTT (SEQ ID NO: 77)
PEPTPEPTPEPTPEPTPEPTT (SEQ ID NO: 78)
PEPTPEPTPEPTPEPTPEPT (SEQ ID NO: 79)
PEPTPEPTPEPTPEPTPEPTS (SEQ ID NO: 80)
PEPTPEPTPEPTPEPTPEPTR (SEQ ID NO: 81)
PPPGGPGGPGTPTSTAPGSGPTSPGGGSG (SEQ ID NO: 82)
PPPGGPGGTGTPTSTAPGSGPTSPGGGSG (SEQ ID NO: 83)
PPSGGPGGPGTPTSTAPGSGPTSPGGGSG (SEQ ID NO: 84)
PEPTPRPTPEPTPRPTG (SEQ ID NO: 85)
PKPTPEPTPKPTPEPTG (SEQ ID NO: 86)
PEPTPKPTPEPTPKPTG (SEQ ID NO: 87)
PEPTPQPTPEPTPQPTG (SEQ ID NO: 88)
PRPTPEPTPRPTG (SEQ ID NO: 89)
PKPTPEPTPKPTG (SEQ ID NO: 90)
PEPTPQPTG (SEQ ID NO: 91)
PEPTPQPTPEPTG (SEQ ID NO: 92)
TPPTPPG (SEQ ID NO: 93)
SPSSPSG (SEQ ID NO: 94)
SPSSPSSPSG (SEQ ID NO: 95)
TPTTPTG (SEQ ID NO: 96)
TPTTPTTPTG (SEQ ID NO: 97)

In a particular embodiment, the linker is PPPPPPP (SEQ ID NO: 31), PPPPPPPG (SEQ ID NO: 30), SPSPSPSPSP (SEQ ID NO: 58) or SPSPSPSPSPG (SEQ ID NO: 25).

In some aspects, a variant of the invention has an improved property relative to a reference enzyme/parent enzyme.

In one aspect, the improved property is increased stability e.g. improved proteolytic stability, improved detergent stability, improved in-wash stability or improved thermostability. In another aspect, the improved property is increased stability during production of the detergent composition or increased performance after storage in the detergent composition relative to the performance of the parent molecule stored at similar conditions. Some aspects of the invention relate to cellulase variants having an improvement factor above 1 when the cellulase variant is tested for a property of interest in a relevant assay, wherein the property of the reference enzyme/parent enzyme is given a value of 1. In some aspects, the property is stability, such as improved proteolytic stability. Some aspects of the invention relate to cellulase variants having an improvement factor above 1 when the cellulase variant is tested for a property of interest in the assay described in Example 2, wherein the property of the reference enzyme/parent enzyme is given a value of 1. In some aspects, the property is stability, such as proteolytic stability.

In some aspects, the improved property is increased stability e.g. improved detergent stability, improved in-wash stability and improved thermostability. Some aspects of the invention relate to cellulase variants having an improvement factor above 1 when the cellulase variant is tested for a property of interest in a relevant assay, wherein the property of the reference enzyme/parent enzyme is given a value of 1, such as when the cellulase variant is tested for a property of interest in the assay described in Example 7.

In some aspects, the improved property is improved thermostability.

In some aspects, the improved property is improved stability in detergent.

In some aspects, the improved property is improved proteolytic stability.

In some aspects, the improved property is one or more or even all of improved thermostability, improved detergent stability, improved proteolytic stability.

A variant according to the invention is improved under the measured conditions when the residual activity ratio, defined as:

Residual Activity Ratio(RAR)=(RA,variant)/(RA,reference)

is above 1.0 compared to the reference cellulase.

In a particularly preferred aspect, a variant according to the invention results in improved stability (e.g., thermostability, detergent stability, proteolytic stability, or more than one or even all of these), where RAR>1.0. In some aspects, the variants according to the invention have a Residual Activity Ratio (RAR) which is at least 1.1; 1.2; 1.3; 1.4; 1.5; 1.6; 1.7; 1.8; 1.9; 2.0; 2.1; 2.2; 2.3; 2.4; 2.5; 2.6; 2.7, 2.8; 2.9; 3.0, 3.1; 3.2; 3.3; 3.4; 3.5, 3.6, 3.7, 3.8, 3.9; 4.0, 4.1; 4.2; 4.3; 4.4; 4.5, 4.6, 4.7, 4.8, 4.9, 5.0, 5.1; 5.2; 5.3; 5.4; 5.5, 5.6, 5.7, 5.8, 5.9; 3.0, 6.1; 6.2; 6.3; 6.4; 6.5, 6.6, 6.7, 6.8, 6.9; 7.0, 7.1; 7.2; 7.3; 7.4; 7.5, 7.6, 7.7, 7.8, 7.9; 8.0, 8.1; 8.2; 8.3; 8.4; 8.5, 8.6, 8.7, 8.8, 8.9; 9.0, 9.1; 9.2; 9.3; 9.4; 9.5, 9.6, 9.7, 9.8, 9.9; 10.0, 10.1; 10.2; 10.3; 10.4; 10.5, 10.6, 10.7, 10.8, 10.9; 12, 15, 16, 20, 25 or 30 compared to a parent or reference enzyme, and in particular compared to a cellulase of SEQ ID NO: 1, SEQ ID NO: 2, SEQ ID NO: 3, or SEQ ID NO: 4.

One preferred embodiment relates to a cellulase variant having improved stability, wherein RAR>1.0, compared to SEQ ID NO: 1. One preferred embodiment relates to a cellulase variant having improved stability, wherein the residual activity ratio (RAR) is at least 1.5, compared to SEQ ID NO: 1, when measured as described in Example 2.

The variants may further comprise one or more additional alterations at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for cellulolytic activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Table B. provides exemplary preferred glycoside hydrolase variants incorporating the CBM variants according to the invention, which are provided in Table form for ease of comparative reference. As used in the Tables herein, the variants are represented in their entirety, in order from N- to C-terminus, with no additional linker or further modification between sequences designated in the respective columns. Thus, Variant 1 represented in the Table as

| | Variant Concept | | |
|---|---|---|---|
| | Catalytic domain | | |
| | (N-terminal) | Linker | CBM (C-terminal) |
| Variant 1 | SEQ ID NO: 5 having | PPPPPPPG (SEQ ID NO: 30) | SEQ ID NO: 173 |
| | mutations A32S S56A | | having mutation |
| | N134D A146D Q147R | | Y14W |
| | Q169Y F183V | | |

could be equally represented as:

(SEQ ID NO: 202)
ASGSGQSTRYWDCCKPSCAWPGKAAVSQPVYSCDANFQRLSDFNVQSGCN
GGSAYACADQTPWAVNDNLAYGFAATSIAGGSESSWCCACYALTFTSGPV
AGKTMVVQSTSTGGDLGSNHFDIAMPGGGVGIFDGCSSQFGGLPGDRYGG
ISSRDQCDSFPAPLKPGCYWRFDWFQNADNPTVTFQQVQCPAEIVARSGC
KRNDDSSFPVFTPPPPPPPGCTTQKWGQCGGIGWTGCTNCVAGTTCTQLN
PWYSQCL

TABLE B
Exemplary glycoside hydrolase variants

| | Variant Concept | | |
|---|---|---|---|
| | Catalytic domain (N-terminal) | Linker | CBM (C-terminal) |
| Variant 1 | SEQ ID NO: 5 having | PPPPPPPG | SEQ ID NO: 173 having mutation |
| | mutations A32S S56A N134D | (SEQ ID NO: 30) | Y14W |
| | A146D Q147R Q169Y F183V | | |
| Variant 2 | SEQ ID NO: 5 having | PPPPPPPG | SEQ ID NO: 173 having mutation |
| | mutations A32S S56A N134D | (SEQ ID NO: 30) | S34H |
| | A146D Q147R Q169Y F183V | | |
| Variant 3 | SEQ ID NO: 5 having | PPPPPPPG | SEQ ID NO: 173 having mutation |
| | mutations A32S S56A N134D | (SEQ ID NO: 30) | S34Y |
| | A146D Q147R Q169Y F183V | | |
| Variant 4 | SEQ ID NO: 5 having | PPPPPPPG | SEQ ID NO: 173 having mutation |
| | mutations A32S S56A N134D | (SEQ ID NO: 30) | S34K |
| | A146D Q147R Q169Y F183V | | |
| Variant 5 | SEQ ID NO: 5 having | PPPPPPPG | SEQ ID NO: 173 having mutation |
| | mutations A32S S56A N134D | (SEQ ID NO: 30) | S34R |
| | A146D Q147R Q169Y F183V | | |
| Variant 6 | SEQ ID NO: 5 having | PPPPPPPG | SEQ ID NO: 173 having mutation |
| | mutations A32S S56A N134D | (SEQ ID NO: 30) | T18K |
| | A146D Q147R Q169Y F183V | | |
| Variant 7 | SEQ ID NO: 5 having | PPPPPPPG | SEQ ID NO: 173 having mutation |
| | mutations A32S S56A N134D | (SEQ ID NO: 30) | L37R |
| | A146D Q147R Q169Y F183V | | |
| Variant 8 | SEQ ID NO: 5 having | PPPPPPPG | SEQ ID NO: 173 having mutations |
| | mutations A32S S56A N134D | (SEQ ID NO: 30) | S34Y, L37R |
| | A146D Q147R Q169Y F183V | | |
| Variant 9 | SEQ ID NO: 5 having | PPPPPPPG | SEQ ID NO: 173 having mutations |
| | mutations A32S S56A N134D | (SEQ ID NO: 30) | T18K, S34H |
| | A146D Q147R Q169Y F183V | | |
| Variant 10 | SEQ ID NO: 5 having | PPPPPPPG | SEQ ID NO: 173 having mutations |
| | mutations A32S S56A N134D | (SEQ ID NO: 30) | S34H, L37R |
| | A146D Q147R Q169Y F183V | | |
| Variant 11 | SEQ ID NO: 5 having | PPPPPPPG | SEQ ID NO: 173 having mutations |
| | mutations A32S S56A N134D | (SEQ ID NO: 30) | T18K, S34Y, L37R |
| | A146D Q147R Q169Y F183V | | |
| Variant 12 | SEQ ID NO: 5 having | PPPPPPPG | SEQ ID NO: 173 having mutation |
| | mutations A32S S56A N134D | (SEQ ID NO: 30) | T18R |
| | A146D Q147R Q169Y F183V | | |
| Variant 13 | SEQ ID NO: 5 having | PPPPPPPG | SEQ ID NO: 173 having mutation |
| | mutations A32S S56A N134D | (SEQ ID NO: 30) | V21E |
| | A146D Q147R Q169Y F183V | | |
| Variant 14 | SEQ ID NO: 5 having | PPPPPPPG | SEQ ID NO: 173 having mutation |
| | mutations A32S S56A N134D | (SEQ ID NO: 30) | A22E |
| | A146D Q147R Q169Y F183V | | |
| Variant 15 | SEQ ID NO: 5 having | PPPPPPPG | SEQ ID NO: 173 having mutation |
| | mutations A32S S56A N134D | (SEQ ID NO: 30) | A22P |
| | A146D Q147R Q169Y F183V | | |
| Variant 16 | SEQ ID NO: 5 having | PPPPPPPG | SEQ ID NO: 173 having mutation |
| | mutations A32S S56A N134D | (SEQ ID NO: 30) | A22N |
| | A146D Q147R Q169Y F183V | | |
| Variant 17 | SEQ ID NO: 5 having | PPPPPPPG | SEQ ID NO: 173 having mutation |
| | mutations A32S S56A N134D | (SEQ ID NO: 30) | T25E |
| | A146D Q147R Q169Y F183V | | |
| Variant 18 | SEQ ID NO: 5 having | PPPPPPPG | SEQ ID NO: 173 having mutation |
| | mutations A32S S56A N134D | (SEQ ID NO: 30) | T27I |
| | A146D Q147R Q169Y F183V | | |
| Variant 19 | SEQ ID NO: 5 having | PPPPPPPG | SEQ ID NO: 173 having mutation |
| | mutations A32S S56A N134D | (SEQ ID NO: 30) | Q28P |
| | A146D Q147R Q169Y F183V | | |
| Variant 20 | SEQ ID NO: 5 having | PPPPPPPG | SEQ ID NO: 173 having mutation |
| | mutations A32S S56A N134D | (SEQ ID NO: 30) | L29P |
| | A146D Q147R Q169Y F183V | | |
| Variant 21 | SEQ ID NO: 5 having | PPPPPPPG | SEQ ID NO: 173 having mutations |
| | mutations A32S S56A N134D | (SEQ ID NO: 30) | S34H, L37R, Y14W |
| | A146D Q147R Q169Y F183V | | |
| Variant 22 | SEQ ID NO: 5 having | PPPPPPPG | SEQ ID NO: 173 having mutations |
| | mutations A32S S56A N134D | (SEQ ID NO: 30) | S34H, L37R, T18K |
| | A146D Q147R Q169Y F183V | | |
| Variant 23 | SEQ ID NO: 5 having | PPPPPPPG | SEQ ID NO: 173 having mutations |
| | mutations A32S S56A N134D | (SEQ ID NO: 30) | S34H, L37R, Y14W, T18K |
| | A146D Q147R Q169Y F183V | | |
| Variant 24 | SEQ ID NO: 5 having | PPPPPPPG | SEQ ID NO: 173 having mutations |
| | mutations A32S S56A N134D | (SEQ ID NO: 30) | S34H, L37R, T18R |
| | A146D Q147R Q169Y F183V | | |
| Variant 25 | SEQ ID NO: 5 having | PPPPPPPG | SEQ ID NO: 173 having mutations |
| | mutations A32S S56A N134D | (SEQ ID NO: 30) | S34H, L37R, V21E |
| | A146D Q147R Q169Y F183V | | |
| Variant 26 | SEQ ID NO: 5 having | PPPPPPPG | SEQ ID NO: 173 having mutations |
| | mutations A32S S56A N134D | (SEQ ID NO: 30) | S34H, L37R, A22E |
| | A146D Q147R Q169Y F183V | | |
| Variant 27 | SEQ ID NO: 5 having | PPPPPPPG | SEQ ID NO: 173 having mutations |
| | mutations A32S S56A N134D | (SEQ ID NO: 30) | S34H, L37R, A22P |
| | A146D Q147R Q169Y F183V | | |
| Variant 28 | SEQ ID NO: 5 having | PPPPPPPG | SEQ ID NO: 173 having mutations |
| | mutations A32S S56A N134D | (SEQ ID NO: 30) | S34H, L37R, A22N |
| | A146D Q147R Q169Y F183V | | |
| Variant 29 | SEQ ID NO: 5 having | PPPPPPPG | SEQ ID NO: 173 having mutations |
| | mutations A32S S56A N134D | (SEQ ID NO: 30) | S34H, L37R, Q28P |
| | A146D Q147R Q169Y F183V | | |
| Variant 30 | SEQ ID NO: 5 having | PPPPPPPG | SEQ ID NO: 173 having mutations |
| | mutations A32S S56A N134D | (SEQ ID NO: 30) | S34H, L37R, L29P |
| | A146D Q147R Q169Y F183V | | |
| Variant 31 | SEQ ID NO: 5 having | PPPPPPPG | SEQ ID NO: 173 having mutations |
| | mutations A32S S56A N134D | (SEQ ID NO: 30) | S34H, L37R, T25E |
| | A146D Q147R Q169Y F183V | | |
| Variant 32 | SEQ ID NO: 5 having | PPPPPPPG | SEQ ID NO: 173 having mutations |
| | mutations A32S S56A N134D | (SEQ ID NO: 30) | S34H, L37R, T27I |
| | A146D Q147R Q169Y F183V | | |
| Variant 33 | SEQ ID NO: 5 having | PPPPPPPG | SEQ ID NO: 173 having mutations |
| | mutations A32S S56A N134D | (SEQ ID NO: 30) | S34H, L37R, V21E, A22E |
| | A146D Q147R Q169Y F183V | | |
| Variant 34 | SEQ ID NO: 5 having | PPPPPPPG | SEQ ID NO: 173 having mutations |
| | mutations A32S S56A N134D | (SEQ ID NO: 30) | S34H, L37R, V21E, A22N |
| | A146D Q147R Q169Y F183V | | |
| Variant 35 | SEQ ID NO: 5 having | PPPPPPPG | SEQ ID NO: 173 having mutations |
| | mutations A32S S56A N134D | (SEQ ID NO: 30) | Y14W, T18R, S34H, L37R |
| | A146D Q147R Q169Y F183V | | |
| Variant 36 | SEQ ID NO: 5 having | PPPPPPPG | SEQ ID NO: 173 having mutations |
| | mutations A32S S56A N134D | (SEQ ID NO: 30) | Y14W, V21E, S34H, L37R |
| | A146D Q147R Q169Y F183V | | |
| Variant 37 | SEQ ID NO: 5 having | PPPPPPPG | SEQ ID NO: 173 having mutations |
| | mutations A32S S56A N134D | (SEQ ID NO: 30) | Y14W, A22E, S34H, L37R |
| | A146D Q147R Q169Y F183V | | |
| Variant 38 | SEQ ID NO: 5 having | PPPPPPPG | SEQ ID NO: 173 having mutations |
| | mutations A32S S56A N134D | (SEQ ID NO: 30) | Y14W, A22P, S34H, L37R |
| | A146D Q147R Q169Y F183V | | |
| Variant 39 | SEQ ID NO: 5 having | PPPPPPPG | SEQ ID NO: 173 having mutations |
| | mutations A32S S56A N134D | (SEQ ID NO: 30) | Y14W, A22N, S34H, L37R |
| | A146D Q147R Q169Y F183V | | |
| Variant 40 | SEQ ID NO: 5 having | PPPPPPPG | SEQ ID NO: 173 having mutations |
| | mutations A32S S56A N134D | (SEQ ID NO: 30) | Y14W, Q18P, S34H, L37R |
| | A146D Q147R Q169Y F183V | | |
| Variant 41 | SEQ ID NO: 5 having | PPPPPPPG | SEQ ID NO: 173 having mutations |
| | mutations A32S S56A N134D | (SEQ ID NO: 30) | Y14W, L29P, S34H, L37R |
| | A146D Q147R Q169Y F183V | | |
| Variant 42 | SEQ ID NO: 5 having | PPPPPPPG | SEQ ID NO: 173 having mutations |
| | mutations A32S S56A N134D | (SEQ ID NO: 30) | Y14W, T25E, S34H, L37R |
| | A146D Q147R Q169Y F183V | | |
| Variant 43 | SEQ ID NO: 5 having | PPPPPPPG | SEQ ID NO: 173 having mutations |
| | mutations A32S S56A N134D | (SEQ ID NO: 30) | Y14W, T27I, S34H, L37R |
| | A146D Q147R Q169Y F183V | | |
| Variant 44 | SEQ ID NO: 5 having | PPPPPPPG | SEQ ID NO: 173 having mutations |
| | mutations A32S S56A N134D | (SEQ ID NO: 30) | Y14W, V21E, A22E, S34H, L37R |
| | A146D Q147R Q169Y F183V | | |
| Variant 45 | SEQ ID NO: 5 having | PPPPPPPG | SEQ ID NO: 173 having mutations |
| | mutations A32S S56A N134D | (SEQ ID NO: 30) | Y14W, V21E, A22N, S34H, L37R |
| | A146D Q147R Q169Y F183V | | |
| Variant 46 | SEQ ID NO: 5 having | PPPPPPPG | SEQ ID NO: 173 having mutations |
| | mutations A32S S56A N134D | (SEQ ID NO: 30) | T18K, V21E, S34H, L37R |
| | A146D Q147R Q169Y F183V | | |
| Variant 47 | SEQ ID NO: 5 having | PPPPPPPG | SEQ ID NO: 173 having mutations |
| | mutations A32S S56A N134D | (SEQ ID NO: 30) | T18K, A22E, S34H, L37R |
| | A146D Q147R Q169Y F183V | | |
| Variant 48 | SEQ ID NO: 5 having | PPPPPPPG | SEQ ID NO: 173 having mutations |
| | mutations A32S S56A N134D | (SEQ ID NO: 30) | T18K, A22P, S34H, L37R |
| | A146D Q147R Q169Y F183V | | |
| Variant 49 | SEQ ID NO: 5 having | PPPPPPPG | SEQ ID NO: 173 having mutations |
| | mutations A32S S56A N134D | (SEQ ID NO: 30) | T18K, A22N, S34H, L37R |
| | A146D Q147R Q169Y F183V | | |
| Variant 50 | SEQ ID NO: 5 having | PPPPPPPG | SEQ ID NO: 173 having mutations |
| | mutations A32S S56A N134D | (SEQ ID NO: 30) | T18K, Q28P, S34H, L37R |
| | A146D Q147R Q169Y F183V | | |
| Variant 51 | SEQ ID NO: 5 having | PPPPPPPG | SEQ ID NO: 173 having mutations |
| | mutations A32S S56A N134D | (SEQ ID NO: 30) | T18K, L29P, S34H, L37R |
| | A146D Q147R Q169Y F183V | | |
| Variant 52 | SEQ ID NO: 5 having | PPPPPPPG | SEQ ID NO: 173 having mutations |
| | mutations A32S S56A N134D | (SEQ ID NO: 30) | T18K, T25E, S34H, L37R |
| | A146D Q147R Q169Y F183V | | |
| Variant 53 | SEQ ID NO: 5 having | PPPPPPPG | SEQ ID NO: 173 having mutations |
| | mutations A32S S56A N134D | (SEQ ID NO: 30) | T18K, T27I, S34H, L37R |
| | A146D Q147R Q169Y F183V | | |
| Variant 54 | SEQ ID NO: 5 having | PPPPPPPG | SEQ ID NO: 173 having mutations |
| | mutations A32S S56A N134D | (SEQ ID NO: 30) | T18K, V21E, A22E, S34H, L37R |
| | A146D Q147R Q169Y F183V | | |
| Variant 55 | SEQ ID NO: 5 having | PPPPPPPG | SEQ ID NO: 173 having mutations |
| | mutations A32S S56A N134D | (SEQ ID NO: 30) | T18K, V21E, A22N, S34H, L37R |
| | A146D Q147R Q169Y F183V | | |
| Variant 56 | SEQ ID NO: 5 having | PPPPPPPG | SEQ ID NO: 173 having mutations |
| | mutations A32S S56A N134D | (SEQ ID NO: 30) | Y14W, T18K, V21E, S34H, L37R |
| | A146D Q147R Q169Y F183V | | |
| Variant 57 | SEQ ID NO: 5 having | PPPPPPPG | SEQ ID NO: 173 having mutations |
| | mutations A32S S56A N134D | (SEQ ID NO: 30) | Y14W, T18K, A22E, S34H, L37R |
| | A146D Q147R Q169Y F183V | | |
| Variant 58 | SEQ ID NO: 5 having | PPPPPPPG | SEQ ID NO: 173 having mutations |
| | mutations A32S S56A N134D | (SEQ ID NO: 30) | Y14W, T18K, A22P, S34H, L37R |
| | A146D Q147R Q169Y F183V | | |
| Variant 59 | SEQ ID NO: 5 having | PPPPPPPG | SEQ ID NO: 173 having mutations |
| | mutations A32S S56A N134D | (SEQ ID NO: 30) | Y14W, T18K, A22N, S34H, L37R |
| | A146D Q147R Q169Y F183V | | |
| Variant 60 | SEQ ID NO: 5 having | PPPPPPPG | SEQ ID NO: 173 having mutations |
| | mutations A32S S56A N134D | (SEQ ID NO: 30) | Y14W, T18K, Q28P, S34H, L37R |
| | A146D Q147R Q169Y F183V | | |
| Variant 61 | SEQ ID NO: 5 having | PPPPPPPG | SEQ ID NO: 173 having mutations |
| | mutations A32S S56A N134D | (SEQ ID NO: 30) | Y14W, T18K, L29P, S34H, L37R |
| | A146D Q147R Q169Y F183V | | |
| Variant 62 | SEQ ID NO: 5 having | PPPPPPPG | SEQ ID NO: 173 having mutations |
| | mutations A32S S56A N134D | (SEQ ID NO: 30) | Y14W, T18K, T25E, S34H, L37R |
| | A146D Q147R Q169Y F183V | | |
| Variant 63 | SEQ ID NO: 5 having | PPPPPPPG | SEQ ID NO: 173 having mutations |
| | mutations A32S S56A N134D | (SEQ ID NO: 30) | Y14W, T18K, T27I, S34H, L37R |
| | A146D Q147R Q169Y F183V | | |
| Variant 64 | SEQ ID NO: 5 having | PPPPPPPG | SEQ ID NO: 173 having mutations |
| | mutations A32S S56A N134D | (SEQ ID NO: 30) | Y14W, T18K, V21E, A22E, S34H, L37R |
| | A146D Q147R Q169Y F183V | | |
| Variant 65 | SEQ ID NO: 5 having | PPPPPPPG | SEQ ID NO: 173 having mutations |
| | mutations A32S S56A N134D | (SEQ ID NO: 30) | Y14W, T18K, A21E, A22N, S34H, L37R |
| | A146D Q147R Q169Y F183V | | |

### Stability in the Presence of Protease

In an embodiment, the variant having glycoside hydrolase activity comprising the CBM variant has improved stability in the presence of a protease compared to the parent enzyme. Preferably the variant has improved stability in the presence of a protease and a surfactant, such as a detergent composition; in comparison with the parent cellulase.

Stability in the presence of protease is beneficial for, e.g. cellulases used under conditions where protease is present, because it extends the time where the cellulases are functional and active and can exert the function they were intended to do.

One preferred use of the variants of the invention is in detergents, where proteases typically are included to improve the detergency. The improved stability of the variants of the invention means that the variants can exert the cellulolytic activity for a longer time during the laundry process compared with the parent cellulase, and thereby provide an improved wash performance benefit compared with the parent cellulase.

For liquid detergent compositions, the variants of the invention further have the benefit that improved stability in the presence of protease means that the liquid detergent composition comprising a protease and further comprising a variant of the invention have a longer shelf life in comparison with the same liquid detergent composition comprising the parent cellulase.

Stability in presence of protease may be determined by incubating a given cellulase under defined conditions in the presence of a protease, measuring the cellulolytic activity after the incubation and comparing it with a sample of the cellulase that has not been incubated with protease.

Another method for determining the stability in presence of protease is to prepare two identical test tubes comprising the given cellulase to be tested in a defined solution comprising a protease, incubating one test tube under elevated temperature e.g. in the range of 30-90° C. (stress) whereas the other tube is incubated at low temperature e.g. in the range of 0-5° C. (non-stress). The tubes are incubated for a predetermined time e.g. between 1 and 24 hours, typically 16 hours. After the incubation both samples are analysed for cellulolytic activity and the residual activity is determined as

Residual activity (%)=(Activity,stress/Activity,non-stress)*100.

For example, it is possible to determine the residual activity in 50% liquid detergent A containing 0.166 v/v-% protease, where the samples are incubated for 16 hours at elevated temperature (stress) and 5° C. (non-stress) before the activity is determined. The temperature should be selected so the residual activity of the parent molecule is in the range of 10-50%.

This core stability method is illustrated in more details in Example 1.

The variants of the invention have higher residual activities than the parent cellulases. In one embodiment, the variants of the invention have at least 10% higher Residual activity compared with the parent cellulase, e.g. at least 20% higher Residual activity, e.g. at least 30% higher Residual activity, e.g. at least 40% higher Residual activity, e.g. at least 50% higher Residual activity, e.g. at least 60% higher Residual activity, e.g. at least 70% higher Residual activity, e.g. at least 80% higher Residual activity, e.g. at least 90% higher Residual activity or at least 100% higher Residual activity, compared with the parent.

However, in the traditional enzyme stability assays used for testing thermostability, activity measures of the stressed and unstressed sample typically focused on measuring changes affecting the catalytic site of the enzyme molecule, e.g. by using a small synthetic substrate such as 4-Methylumbelliferyl-β-cellopentaoside or soluble carboxymethyl cellulose (CMC).

Importantly, however, changes in other properties of the enzyme of interest due to the stress, properties important for the function of the enzyme in the application but not directly affecting the active site of the enzyme, is not necessarily detected in these assays. One such example is the glycosyl hydrolases having a separate catalytic domain and a CBM joined by a linker as e.g. in cellulases used for removing fuzz and pills in laundry detergents and textile care products. If the stress affects only the linker and/or CBM part of the molecule but not the catalytic domain part, these changes will not be detected by the traditional assays as described above and/or in Example 1. The activity, when using a simple substrate such as CMC or 4-methylumbelliferyl-β-cellopentaoside, will appear to be maintained during the stress but the performance is significantly affected, as the CBM part of the enzyme molecule plays an important role in directing the enzyme to the proper location on the textile to be treated.

As an alternative, the importance of the CBM for the performance can be tested by comparing the performance of the catalytic domain with that of the catalytic domain with intact linker and CBM.

To detect changes in the linker and/or CBM after storage under stressed conditions special measures must be taken when testing if the stress has affected the performance of the enzyme. This can be done by comparing the performance of the enzymes before and after stress. Alternatively, it can be tested by ensuring that binding of the enzyme to its natural, insoluble substrate, such as cotton linters, is included as part of the assay used for testing the stability, and/or first probing the binding of the enzyme to microcrystalline cellulose or cotton linters and then measure the activity of the enzymes having lost their binding ability to the cellulose compared to the total activity.

Thus, linker and/or CBM stability is measured by incubating the cellulase in detergent containing protease, followed by determining the ability of the incubated cellulase to bind to cellulose fibers. If the linker or the cellulose binding domain is affected by the protease the binding affinity of the cellulase to cellulose fibers will be reduced.

This linker and CBM specific assay are illustrated by the conditions described in Example 2.

The parent polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly. The parent may be a bacterial cellulase. For example, the parent may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces cellulase, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma cellulase.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis cellulase.

In another aspect, the parent is a Streptococcus equisimilis, Streptococcus pyogenes, Streptococcus uberis, or Streptococcus equi subsp. Zooepidemicus cellulase.

In another aspect, the parent is a Streptomyces achromogenes, Streptomyces avermitilis, Streptomyces coelicolor, Streptomyces griseus, or Streptomyces lividans cellulase.

The parent may be a fungal cellulase. For example, the parent may be a yeast cellulase such as a Candida, Kluyveromyces, Pichia, Saccharomyces, Schizosaccharomyces, or Yarrowia cellulase; or a filamentous fungal cellulase such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria cellulase.

In another aspect, the parent is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis cellulase.

In another aspect, the parent is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride cellulase.

In another aspect, the parent is a Thielavia terrestris cellulase, e.g., the cellulase of SEQ ID NO: 1 or the mature polypeptide thereof.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

The present invention also relates to methods for obtaining a variant having glycoside hydrolase activity, comprising: (a) introducing into a parent glycoside hydrolase one or more substitutions of the mature polypeptide of the parent polypeptide at positions corresponding to positions 14, 18, 21, 22, 25, 27, 28, 29, 34 and/or 37 of the CBM1 using SEQ ID NO: 173 for alignment; and (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a variant of the present invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (VillaKamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alphaamylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus nigerglucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid-state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Detergent Compositions

In one embodiment, the invention is directed to detergent compositions comprising an enzyme of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment, the invention is directed to a liquid laundry detergent composition comprising an enzyme of the present invention in combination with one or more additional laundry detergent composition components, specifically a protease. In another embodiment, the invention comprises an ancillary product used in laundry, such as a prespotter or stain removal booster. The present invention also relates to an ADW (Automatic Dish Wash) compositions comprising an enzyme of the present invention in combination with one or more additional ADW composition components.

The choice of additional components is within the skill of the artisan and includes conventional ingredients, including the exemplary non-limiting components set forth below.

In one embodiment of the present invention, the variant having glycoside hydrolase activity comprising a CBM1 variant of the present invention may be added to a detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The enzyme(s) of the detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

A polypeptide of the present invention may also be incorporated in the detergent formulations disclosed in WO97/07202, which is hereby incorporated by reference.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 5% to 60% by weight, such as about 5% to about 50%, or about 10% to about 50%, or about 20% to about 50%. The surfactant(s) is chosen based on the desired cleaning application, and may include any conventional surfactant(s) known in the art.

When included therein the detergent will usually contain from about 5% to about 60% by weight of one or more anionic surfactants, such as from about 5% to about 40%, including from about 10% to about 25%, Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salts of fatty acids (soap) or fatty acids, and combinations thereof.

When included therein the detergent will usually contain from about from about 0,1% to about 10% by weigh of a cationic surfactant, for example from about 0.1% to about 5%, Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 60% by weight of a nonionic surfactant, for example from about 1% to about 40%, in particular from about 5% to about 20%, from about 3% to about 15%, Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), methylester ethoxylates (MEE), as well as products available under the trade names SPAN and TWEEN, and combinations thereof.

When included therein the detergent will usually contain from about 0,1% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0,1% to about 10% by weight of a zwitterionic surfactant. Non-limiting examples of zwitterionic surfactants include betaines such as alkyldimethylbetaines, sulfobetaines, and combinations thereof.

Solvent system: For dissolution of the surfactant and other detergent ingredients, a solvent system is needed. Solvents are typically water, alcohols, polyols, sugars and/or mixtures thereof. Preferred solvents are water, glycerol, sorbitol, propylene glycol (MPG, 1,2-propanediol or 1,3-propane diol), dipropylene glycol (DPG), polyethylene glycol family (PEG300-600), hexylene glycol, inositol, mannitol, Ethanol, isopropanol, n-butoxy propoxy propanol, ethanolamines (monoethanol amine, diethanol amines and triethanol amines), sucrose, dextrose, glucose, ribose, xylose, and related mono and di pyranosides and furanosides.

The solvent system is present in typically totally 5-90%, 5-60%, 5-40%, 10-30% by weight.

The water content for unit doses wrapped in PVA film is typically in the range 1-15%, 2-12%, 3-10%, 5-10%.

The polyol content for unit doses wrapped in PVA film is typically in the range 5-50%, 10-40% or 20-30%.

In an embodiment, the surfactant is a non-naturally occurring surfactant.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants), however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming micellar, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65%, 0-20%; or 0.5-5% of a detergent builder or co-builder, or a mixture thereof. In a dish wash detergent, the level of builder is typically 10-65%, particularly 20-40%. The builder and/or co-builder may particularly be a chelating agent that forms water-soluble complexes with Ca and Mg. Any builder and/or co-builder known in the art for use in laundry detergents may be utilized. Nonlimiting examples are citrate, sodium carbonate, sodium bicarbonate and sodium citrate, Examples of phosphonates include 1-Hydroxy Ethylidene-1,1-Diphosphonic Acid (HEDP, etidronic acid), Diethylenetriamine Penta(Methylene Phosphonic acid) (DTPMP), Ethylene diamine tetra(methylene phosphonic acid) (EDTMPA), amino tris(methylenephosphonic acid) (ATMP), Nitrilo trimethylene phosphonic acid (NTMP), 2-Amino ethyl phosphonic acid (AEPn), Dimethyl methylphosphonate (DMPP),Tetramethylene diamine tetra(methylene phosphonic acid) (TDTMP), Hexamethylene diamine tetra(methylene phosphonic acid) (HDTMP), Phosphonobutane-tricarboxylic acid (PBTC), N-(phosphonomethyl) iminodiacetic acid (PMIDA), 2-carboxyethyl phosphonic acid (CEPA), 2-Hydroxy phosphonocarboxylic acid (HPAA) and Amino-tris-(methylene-phosphonic acid) (AMP). L-glutamic acid N,N-diacetic acid tetra sodium salt (GLDA), methylglycinediacetic acid (MGDA). Non-limiting examples of builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamineN,N′N-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

In an embodiment, the builder or co-builder is a non-naturally occurring builder or co-builder.

### Bleaching Systems

The detergent may contain 0-30% by weight, such as about 1% to about 20%, of a bleaching system. Any bleaching system known in the art for use in laundry detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally, ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

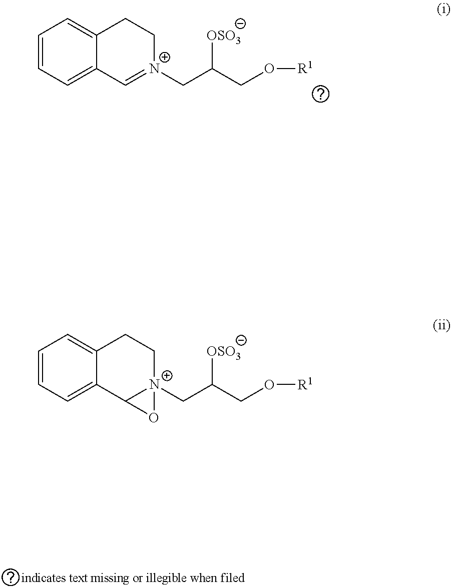

(iii) and mixtures thereof,

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) preformed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

In an embodiment, the bleaching system is a non-naturally occurring bleaching system.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or antifoaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PETPOET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

In an embodiment, the polymer is a non-naturally occurring polymer.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Color Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Additional Enzymes

The detergent additive as well as the detergent composition may comprise one or more [additional] enzymes such as hydrolases (EC 3.-.-.-) such as hydrolases acting on ester bonds (EC 3.1.-.-), glycosidases (EC 3.2.-.-), and hydrolases acting on peptide bonds (EC 3.4.-.-), oxidoreductases (EC 1.-.-.-) such as laccases (EC 1.10.-.-) or peroxidases (EC 1.11.-.-) or lyases (EC 4.-.-.-) such as carbon-oxygen lyases (EC 4.2.-.-). In a specific embodiment the detergent composition may comprise one or more [additional] enzymes such as a protease, lipase, cutinase, an amylase, carbohydrase, cellulase, pectinase, mannanase, arabinase, galactanase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases providing or maintaining whiteness and preventing redeposition or having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Eng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167,170, 194, 195, 199, 205, 206, 217, 218, 222, 224, 232, 235, 236, 245, 248, 252 and 274 using the BPN′ numbering. More preferred the subtilase variants may comprise the mutations: S3T, V41, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering).

Suitable commercially available protease enzymes include those sold under the trade names Alcalase®, Blaze®, Blaze® Evity®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® Esperase®, Progress Excel®, and Progress Uno® (Novozymes A/S), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases:

Suitable amylases which can be used together with the variants of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T491+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™ Stainzyme PIus™, Natalase™ and BAN™ (from Novozymes A/S), and Rapidase™, Purastar™/Effectenz™, Powerase™, Preferenz S1000™ Preferenz S110™ and Preferenz S100™ (from Genencor International Inc./DuPont).

### Peroxidases/Oxidases:

A peroxidase is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase may also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase can include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Nucleases

Suitable nucleases include deoxyribonucleases (DNases) and ribonucleases (RNases) which are any enzyme that catalyzes the hydrolytic cleavage of phosphodiester linkages in the DNA or RNA backbone respectively, thus degrading DNA and RNA. There are two primary classifications based on the locus of activity. Exonucleases digest nucleic acids from the ends. Endonucleases act on regions in the middle of target molecules. The nuclease is preferably a DNase, which is preferable is obtainable from a microorganism, preferably a fungi or bacterium. In particular, a DNase which is obtainable from a species of Bacillus is preferred; in particular a DNase which is obtainable from Bacillus cibi, Bacillus subtilis or Bacillus licheniformis is preferred. Examples of such DNases are described in WO 2011/098579, WO2014/087011 and WO2017/060475. Particularly preferred is also a DNase obtainable from a species of Aspergillus; in particular a DNase which is obtainable from Aspergillus oryzae, such as a DNase described in WO 2015/155350.

### Licheninases

Suitable licheninases (lichenases) include enzymes that catalyse the hydrolysis of the beta-1,4-glucosidic bonds to give beta-glucans. Licheninases (or lichenases) (e.g. EC 3.2.1.73) hydrolyse (1,4)-beta-D-glucosidic linkages in beta-D-glucans containing (1,3)- and (1,4)-bonds and can act on lichenin and cereal beta-D-glucans, but not on beta-D-glucans containing only 1,3- or 1,4-bonds. Examples of such licheninases are described in patent application WO 2017/097866 and in WO 2017/129754.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants—The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent—The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbenesulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Soil release polymers—The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents—The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Rheology Modifiers—are structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, antiwrinkling agents, bactericides, binders, carriers, dyes, enzyme stabilizers, fabric softeners, fillers, foam regulators, hydrotropes, perfumes, pigments, sod suppressors, and solvents.

### Protease Inhibitor

The protease inhibitor maybe any compound which stabilises or inhibits the protease so that the protease or other enzyme(s) in the laundry soap bar are not degraded. Examples of protease inhibitors are aprotinin, bestatin, calpain inhibitor I and II, chymostatin, leupeptin, pepstatin, phenylmethanesulfonyl fluoride (PMSF), boric acid, borate, borax, boronic acids, phenylboronic acids such as 4-formylphenylboronic acid (4-FPBA), peptide aldehydes or hydrosulfite adducts or hemiacetal adducts thereof and peptide trifluromethyl ketones. There may be one or more protease inhibitors, such as 5,4,3,2 or 1 inhibitor(s) of which at least one is a peptide aldehyde, a hydrosulfite adduct or a hemiacetal adduct thereof.

Peptide Aldehyde Inhibitor

The peptide aldehyde may have the formula P-(A)_{y}-L-(B)ₓ—B⁰—H or a hydrosulfite adduct or hemiacetal adduct thereof, wherein:
i. H is hydrogen;
ii. B⁰ is a single amino acid residue with L- or D-configuration of the formula —NH—CH(R)—C(═O)—;
iii. x is 1, 2 or 3 for (B)ₓ, and B is independently a single amino acid connected to B° via the C-terminal of the B amino acid
iv. L is absent or L is independently a linker group of the formula —C(═O)—, —C(═O)—C(═O)—, —C(═S)—, —C(═S)—C(═S)— or —C(═S)—C(═O)—;
v. y is 0, 1 or 2 for (A)_{y}, and A is independently a single amino acid residue connected to L via the N-terminal of the A amino acid, with the proviso that if L is absent then A is absent;
vi. P is selected from the group consisting of hydrogen and an N-terminal protection group, with the proviso that if L is absent then P is an N-terminal protection group;
vii. R is independently selected from the group consisting of C₁₋₆ alkyl, C₆₋₁₀ aryl or C₇₋₁₀ arylalkyl optionally substituted with one or more, identical or different, substituent's R′;
viii. R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂; and
ix. R″ is a C₁₋₆ alkyl group.

x may be 1, 2 or 3 and therefore B may be 1, 2 or 3 amino acid residues respectively. Thus, B may represent B¹, B²—B¹ or B³—B²—B¹, where B³, B² and B¹ each represent one amino acid residue. y may be 0, 1 or 2 and therefore A may be absent, or 1 or 2 amino acid residues respectively having the formula A¹ or A²-A¹ wherein A² and A¹ each represent one amino acid residue.

B⁰ may be a single amino acid residue with L- or D-configuration, which is connected to H via the C-terminal of the amino acid, wherein R is a C₁₋₆ alkyl, C₆₋₁₀ aryl or C₇₋₁₀ arylalkyl side chain, such as methyl, ethyl, propyl, isopropyl, butyl, isobutyl, phenyl or benzyl, and wherein R may be optionally substituted with one or more, identical or different, substituent's R′. Particular examples are the D- or L-form of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr) and valine (Val). A particular embodiment is when B⁰ is leucine, methionine, phenylalanine, p-tyrosine and valine.

B¹, which is connected to B⁰ via the C-terminal of the B¹ amino acid, may be an aliphatic, hydrophobic and/or neutral amino acid. Examples of B¹ are alanine (Ala), cysteine (Cys), glycine (Gly), isoleucine (Ile), leucine (Leu), norleucine (Nle), norvaline (Nva), proline (Pro), serine (Ser), threonine (Thr) and valine (Val). Particular examples of B¹ are alanine, glycine, isoleucine, leucine and valine. A particular embodiment is when B¹ is alanine, glycine or valine.

If present, B², which is connected to B¹ via the C-terminal of the B² amino acid, may be an aliphatic, hydrophobic, neutral and/or polar amino acid. Examples of B² are alanine (Ala), arginine (Arg), capreomycidine (Cpd), cysteine (Cys), glycine (Gly), isoleucine (Ile), leucine (Leu), norleucine (Nle), norvaline (Nva), phenylalanine (Phe), proline (Pro), serine (Ser), threonine (Thr), and valine (Val). Particular examples of B² are alanine, arginine, capreomycidine, glycine, isoleucine, leucine, phenylalanine and valine. A particular embodiment is when B² is arginine, glycine, leucine, phenylalanine or valine.

B³, which if present is connected to B² via the C-terminal of the B³ amino acid, may be a large, aliphatic, aromatic, hydrophobic and/or neutral amino acid. Examples of B³ are isoleucine (Ile), leucine (Leu), norleucine (Nle), norvaline (Nva), phenylalanine (Phe), phenylglycine, tyrosine (Tyr), tryptophan (Trp) and valine (Val). Particular examples of B³ are leucine, phenylalanine, tyrosine and tryptophan.

The linker group L may be absent or selected from the group consisting of —C(═O)—, —C(═O)—C(═O)—, —C(═S)—, —C(═S)—C(═S)— or —C(═S)—C(═O)—. Particular embodiments include when L is absent or L is a carbonyl group —C(═O)—.

A¹, which if present is connected to L via the N-terminal of the amino acid, may be an aliphatic, aromatic, hydrophobic, neutral and/or polar amino acid. Examples of A¹ are alanine (Ala), arginine (Arg), capreomycidine (Cpd), glycine (Gly), isoleucine (Ile), leucine (Leu), norleucine (Nle), norvaline (Nva), phenylalanine (Phe), threonine (Thr), tyrosine (Tyr), tryptophan (Trp) and valine (Val). Particular examples of A¹ are alanine, arginine, glycine, leucine, phenylalanine, tyrosine, tryptophan and valine. A particular embodiment is when B² is leucine, phenylalanine, tyrosine or tryptophan.

The A² residue, which if present is connected to A¹ via the N-terminal of the amino acid, may be a large, aliphatic, aromatic, hydrophobic and/or neutral amino acid. Examples of A² are arginine (Arg), isoleucine (Ile), leucine (Leu), norleucine (Nle), norvaline (Nva), phenylalanine (Phe), phenylglycine, Tyrosine (Tyr), tryptophan (Trp) and valine (Val). Particular examples of A² are phenylalanine and tyrosine.

The N-terminal protection group P (if present) may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl, (fluoromethoxy)carbonyl, benzyloxycarbonyl (Cbz), t-butyloxycarbonyl (Boc) and adamantyloxycarbonyl; p-methoxybenzyl carbonyl (Moz), benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP(OH)(═O)) and benzylphosphoramidyl (PhCH₂OP(OH)(═O)).

The general formula of the peptide aldehyde may also be written: P-A²-A¹-L-B³—B² B¹—B⁰—H, where P, A², A¹,L, B³, B², B¹ and B⁰ are as defined above.

In the case of a tripeptide aldehyde with a protection group (i.e. x=2, L is absent and A is absent), P is preferably acetyl, methoxycarbonyl, benzyloxycarbonyl, methylamino carbonyl, methylsulfonyl, benzylsulfonyl and benzylphosphoramidyl. In the case of a tetrapeptide aldehyde with a protection group (i.e. x=3, L is absent and A is absent), P is preferably acetyl, methoxycarbonyl, methylsulfonyl, ethylsulfonyl and methylphosphoramidyl.

Suitable peptide aldehydes are described in WO94/04651, WO95/25791, WO98/13458, WO98/13459, WO98/13460, WO98/13461, WO98/13462, WO07/141736, WO07/145963, WO09/118375, WO10/055052 and WO11/036153.

More particularly, the peptide aldehyde may be

Cbz-Arg-Ala-Tyr-H (L-Alaninamide, N2-[(phenylmethoxy)carbonyl]-L-arginyl-N-[(1S)-1-formyl-2-(4-hydroxyphenyl)ethyl]-),

Ac-Gly-Ala-Tyr-H (L-Alaninamide, N-acetylglycyl-N-[(1S)-1-formyl-2-(4-hydroxyphenyl)ethyl]-)

Cbz-Gly-Ala-Tyr-H (L-Alaninamide, N-[(phenylmethoxy)carbonyl]glycyl-N-[(1S)-1-formyl-2-(4-hydroxyphenyl)ethyl]-),

Cbz-Gly-Ala-Leu-H (L-Alaninamide, N-[(phenylmethoxy)carbonyl]glycyl-N-[(1S)-1-formyl-3-methylbutyl]-),

Cbz-Val-Ala-Leu-H (L-Alaninamide, N-[(phenylmethoxy)carbonyl]-L-valyl-N-[(1S)-1-formyl-3-methylbutyl]-),

Cbz-Gly-Ala-Phe-H (L-Alaninamide, N-[(phenylmethoxy)carbonyl]glycyl-N-[(1S)-1-formyl-2-phenylethyl]-),

Cbz-Gly-Ala-Val-H (L-Alaninamide, N-[(phenylmethoxy)carbonyl]glycyl-N-[(1S)-1-formyl-2-methylpropyl]-),

Cbz-Gly-Gly-Tyr-H (Glycinamide, N-[(phenylmethoxy)carbonyl]glycyl-N-[(1S)-1-formyl-2-(4-hydroxyphenyl)ethyl]-),

Cbz-Gly-Gly-Phe-H (Glycinamide, N-[(phenylmethoxy)carbonyl]glycyl-N-[(1S)-1-formyl-2-phenylethyl]-),

Cbz-Arg-Val-Tyr-H (L-Valinamide, N2-[(phenylmethoxy)carbonyl]-L-arginyl-N-[(1S)-1-formyl-2-(4-hydroxyphenyl)ethyl]-),

Cbz-Leu-Val-Tyr-H (L-Valinamide, N-[(phenylmethoxy)carbonyl]-L-leucyl-N-[(1S)-1-formyl-2-(4-hydroxyphenyl)ethyl]-)

Ac-Leu-Gly-Ala-Tyr-H (L-Alaninamide, N-acetyl-L-leucylglycyl-N-[(1S)-1-formyl-2-(4-hydroxyphenyl)ethyl]-),

Ac-Phe-Gly-Ala-Tyr-H (L-Alaninamide, N-acetyl-L-phenylalanylglycyl-N-[(1S)-1-formyl-2-(4-hydroxyphenyl)ethyl]-),

Ac-Tyr-Gly-Ala-Tyr-H (L-Alaninamide, N-acetyl-L-tyrosylglycyl-N-[(1S)-1-formyl-2-(4-hydroxyphenyl)ethyl]-),

Ac-Phe-Gly-Ala-Leu-H (L-Alaninamide, N-acetyl-L-phenylalanylglycyl-N-[(1S)-1-formyl-3-methylbutyl]-),

Ac-Phe-Gly-Ala-Phe-H (L-Alaninamide, N-acetyl-L-phenylalanylglycyl-N-[(1S)-1-formyl-2-phenylethyl]-)

Ac-Phe-Gly-Val-Tyr-H (L-Valinamide, N-acetyl-L-phenylalanylglycyl-N-[(1S)-1-formyl-2-(4-hydroxyphenyl)ethyl]-),

Ac-Phe-Gly-Ala-Met-H (L-Alaninamide, N-acetyl-L-phenylalanylglycyl-N-[(1S)-1-formyl-3-(methylthio)propyl]-),

Ac-Trp-Leu-Val-Tyr-H (L-Valinamide, N-acetyl-L-tryptophyl-L-leucyl-N-[(1S)-1-formyl-2-(4-hydroxyphenyl)ethyl]-),

MeO—CO-Val-Ala-Leu-H (L-Alaninamide, N-(methoxycarbonyl)-L-valyl-N-[(1S)-1-formyl-3-methylbutyl]-)

MeNHCO-Val-Ala-Leu-H (L-Alaninamide, N-(aminomethylcarbonyl)-L-valyl-N-[(1S)-1-formyl-3-methylbutyl]-),

MeO—CO-Phe-Gly-Ala-Leu-H (L-Alaninamide, N-(methoxycarbonyl)-L-phenylalanylglycyl-N-[(1S)-1-formyl-3-methylbutyl]-),

MeO—CO-Phe-Gly-Ala-Phe-H (L-Alaninamide, N-(methoxycarbonyl)-L-phenylalanylglycyl-N-[(1S)-1-formyl-2-phenylethyl]-),

MeSO2-Phe-Gly-Ala-Leu-H (L-Alaninamide, N-(methylsulfonyl)-L-phenylalanylglycyl-N-[(1S)-1-formyl-3-methylbutyl]-),

MeSO2-Val-Ala-Leu-H (L-Alaninamide, N-(methylsulfonyl)-L-valyl-N-[(1S)-1-formyl-3-methylbutyl]-),

PhCH2O—P(OH)(O)-Val-Ala-Leu-H (L-Alaninamide, N-[hydroxy(phenylmethoxy)phosphinyl]-L-valyl-N-[(1S)-1-formyl-3-methylbutyl]-),

EtSO2-Phe-Gly-Ala-Leu-H (L-Alaninamide, N-(ethylsulfonyl)-L-phenylalanylglycyl-N-[(1S)-1-formyl-3-methylbutyl]-),

PhCH2SO2-Val-Ala-Leu-H (L-Alaninamide, N-[(phenylmethyl)sulfonyl]-L-valyl-N-[(1S)-1-formyl-3-methylbutyl]-),

PhCH2O—P(OH)(O)-Leu-Ala-Leu-H (L-Alaninamide, N-[hydroxy(phenylmethoxy)phosphinyl]-L-leucyl-N-[(1S)-1-formyl-3-methylbutyl]-),

PhCH2O—P(OH)(O)-Phe-Ala-Leu-H (L-Alaninamide, N-[hydroxy(phenylmethoxy)phosphinyl]-L-phenylalanyl-N-[(1S)-1-formyl-3-methylbutyl]-), or

MeO—P(OH)(O)-Leu-Gly-Ala-Leu-H; (L-Alaninamide, N-(hydroxymethoxyphosphinyl)-Lleucylglycyl-N-[(1S)-1-formyl-3-methylbutyl]-).

A preferred example is Cbz-Gly-Ala-Tyr-H.

Further examples of such peptide aldehydes include

α-MAPI (3,5,8,11-Tetraazatridecanoic acid, 6-[3-[(aminoiminomethyl)amino]propyl]-12-formyl-9-(1-methylethyl)-4,7,10-trioxo-13-phenyl-2-(phenylmethyl)-, (2S,6S,9S,12S)-

L-Valinamide, N2-[[(1-carboxy-2-phenylethyl)amino]carbonyl]-L-arginyl-N-(1-formyl-2-phenylethyl)-, [1(S),2(S)]-; L-Valinamide, N2-[[[(1S)-1-carboxy-2-phenylethyl]amino]carbonyl]-Larginyl-N-[(1S)-1-formyl-2-phenylethyl]-(9Cl); SP-Chymostatin B),

β-MAPI (L-Valinamide, N2-[[[(1S)-1-carboxy-2-phenylethyl]amino]carbonyl]-L-arginyl-N-[(1R)-1-formyl-2-phenylethyl]-L-Valinamide, N2-[[(1-carboxy-2-phenylethyl)amino]carbonyl]-L-arginyl-N-(1-formyl-2-phenylethyl)-, [1(S),2(R)]-),

Phe-C(═O)—Arg-Val-Tyr-H (L-Valinamide, N2-[[[(1S)-1-carboxy-2-phenylethyl]amino]carbonyl]-L-arginyl-N-[(1S)-1-formyl-2-(4-hydroxyphenyl)ethyl]-(9Cl)),

Phe-C(═O)-Gly-Gly-Tyr-H, (3,5,8,11-Tetraazatridecanoic acid, 12-formyl-13-(4-hydroxyphenyl)-4,7,10-trioxo-2-(phenylmethyl)-, (2S,12S)-),

Phe-C(═O)-Gly-Ala-Phe-H, (3,5,8,11-Tetraazatridecanoic acid, 12-formyl-9-methyl-4,7,10-trioxo-13-phenyl-2-(phenylmethyl)-, (2S,9S,12S)-),

Phe-C(═O)-Gly-Ala-Tyr-H (3,5,8,11-Tetraazatridecanoic acid, 12-formyl-13-(4-hydroxyphenyl)-9-methyl-4,7,10-trioxo-2-(phenylmethyl)-, (2S,9S,12S)-),

Phe-C(═O)-Gly-Ala-Leu-H, (3,5,8,11-Tetraazapentadecanoic acid, 12-formyl-9,14-dimethyl-4,7,10-trioxo-2-(phenylmethyl)-, (2S,9S,12S)-),

Phe-C(═O)-Gly-Ala-Nva-H, (3,5,8,11-Tetraazapentadecanoic acid, 12-formyl-9-methyl-4,7,10-trioxo-2-(phenylmethyl)-, (2S,9S,12S)-),

Phe-C(═O)-Gly-Ala-Nle-H (3,5,8,11-Tetraazahexadecanoic acid, 12-formyl-9-methyl-4,7,10-trioxo-2-(phenylmethyl)-, (2S,9S,12S)-),

Tyr-C(═O)—Arg-Val-Tyr-H (L-Valinamide, N2-[[[(1S)-1-carboxy-2-(4-hydroxyphenyl)ethyl]amino]carbonyl]-L-arginyl-N-[(1S)-1-formyl-2-(4-hydroxyphenyl)ethyl]-(9Cl))

Tyr-C(═O)-Gly-Ala-Tyr-H (3,5,8,11-Tetraazatridecanoic acid, 12-formyl-13-(4-hydroxyphenyl)-2-[(4-hydroxyphenyl)methyl]-9-methyl-4,7,10-trioxo-, (2S,9S,12S)-)

Phe-C(═S)—Arg-Val-Phe-H, (3,5,8,11-Tetraazatridecanoic acid, 6-[3-[(aminoiminomethyl)amino]propyl]-12-formyl-9-(1-methylethyl)-7,10-dioxo-13-phenyl-2-(phenylmethyl)-4-thioxo-, (2S,6S,9S,12S)-),

Phe-C(═S)—Arg-Val-Tyr-H, (3,5,8,11-Tetraazatridecanoic acid, 6-[3-[(aminoiminomethyl)amino]propyl]-12-formyl-13-(4-hydroxyphenyl)-9-(1-methylethyl)-7,10-dioxo-2-(phenylmethyl)-4-thioxo-, (2S,6S,9S,12S)-),

Phe-C(═S)-Gly-Ala-Tyr-H, (3,5,8,11-Tetraazatridecanoic acid, 12-formyl-13-(4-hydroxyphenyl)-9-methyl-7,10-dioxo-2-(phenylmethyl)-4-thioxo-, (2S,9S,12S)-),

Antipain (L-Valinamide, N2-[[(1-carboxy-2-phenylethyl)amino]carbonyl]-L-arginyl-N-[4-[(aminoiminomethyl)amino]-1-formylbutyl]-),

GE20372A (L-Valinamide, N2-[[[(1S)-1-carboxy-2-(4-hydroxyphenyl)ethyl]amino]carbonyl]-L-arginyl-N-[(1S)-1-formyl-2-phenylethyl]-

L-Valinamide, N2-[[[1-carboxy-2-(4-hydroxyphenyl)ethyl]amino]carbonyl]-L-arginyl-N-(1-formyl-2-phenylethyl)-, [1(S),2(S)]-),

GE20372B (L-Valinamide, N2-[[[(1S)-1-carboxy-2-(4-hydroxyphenyl)ethyl]amino]carbonyl]-L-arginyl-N-[(1R)-1-formyl-2-phenylethyl]-

L-Valinamide, N2-[[[1-carboxy-2-(4-hydroxyphenyl)ethyl]amino]carbonyl]-L-arginyl-N-(1-formyl-2-phenylethyl)-, [1(S),2(R)]-),

Chymostatin A (L-Leucinamide, (2S)-2-[(4S)-2-amino-3,4,5,6-tetrahydro-4-pyrimidinyl]-N-[[[(1S)-1-carboxy-2-phenylethyl]amino]carbonyl]glycyl-N-(1-formyl-2-phenylethyl)-

L-Leucinamide, (2S)-2-[(4S)-2-amino-1,4,5,6-tetrahydro-4-pyrimidinyl]-N-[[[(1S)-1-carboxy-2-phenylethyl]amino]carbonyl]glycyl-N-(1-formyl-2-phenylethyl)-(9Cl); L-Leucinamide, L-2-(2-amino-1,4,5,6-tetrahydro-4-pyrimidinyl)-N-[[(1-carboxy-2-phenylethyl)amino]carbonyl]glycyl-N-(1-formyl-2-phenylethyl)-, stereoisomer),

Chymostatin B (L-Valinamide, (2S)-2-[(4S)-2-amino-3,4,5,6-tetrahydro-4-pyrimidinyl]-N-[[[(1S)-1-carboxy-2-phenylethyl]amino]carbonyl]glycyl-N-(1-formyl-2-phenylethyl)-

L-Valinamide, (2S)-2-[(4S)-2-amino-1,4,5,6-tetrahydro-4-pyrimidinyl]-N-[[[(1S)-1-carboxy-2-phenylethyl]amino]carbonyl]glycyl-N-(1-formyl-2-phenylethyl)-(9Cl); L-Valinamide, L-2-(2-amino-1,4,5,6-tetrahydro-4-pyrimidinyl)-N-[[(1-carboxy-2-phenylethyl)amino]carbonyl]glycyl-N-(1-formyl-2-phenylethyl)-, stereoisomer), and

Chymostatin C (L-Isoleucinamide, (2S)-2-[(4S)-2-amino-3,4,5,6-tetrahydro-4-pyrimidinyl]-N-[[[(1S)-1-carboxy-2-phenylethyl]amino]carbonyl]glycyl-N-(1-formyl-2-phenylethyl)-

L-Isoleucinamide, (2S)-2-[(4S)-2-amino-1,4,5,6-tetrahydro-4-pyrimidinyl]-N-[[[(1S)-1-carboxy-2-phenylethyl]amino]carbonyl]glycyl-N-(1-formyl-2-phenylethyl)-(9Cl); L-Isoleucinamide,

L-2-(2-amino-1,4,5,6-tetrahydro-4-pyrimidinyl)-N-[[(1-carboxy-2-phenylethyl)amino]carbonyl]glycyl-N-(1-formyl-2-phenylethyl)-, stereoisomer).

Peptide Aldehyde Adducts

Instead of a peptide aldehyde, the protease inhibitor may be an adduct of a peptide aldehyde. The adduct maybe a hydrosulfite adduct having the formula P-(A)_{y}-L-(B)ₓ—N(H)—CHR—CH(OH)—SO₃M, wherein P, A, y, L, B, x and R are defined as above, and M is H or an alkali metal, preferably Na or K. Alternatively, the adduct may be a hemiacetal having the formula P-(A)_{y}-L(B)ₓ—N(H)—CHR—CH(OH)—OR, wherein P, A, y, L, B, x and R are defined as above. A preferred embodiment is a hydrosulfite adduct wherein P=Cbz, B²=Gly; B¹=Ala; B⁰=Tyr (so R=PhCH₂, R′═OH), x=2, y=0, L=A=absent and M=Na (Cbz-Gly-Ala-N(H)—CH(CH₂-p-C₆H₄OH)—CH(OH)—SO₃Na, L-Alaninamide, N-[(phenylmethoxy)carbonyl]glycyl-N-[2-hydroxy-1-[(4-hydroxyphenyl)methyl]-2-sulfoethyl]-, sodium salt (1:1)).

The general formula of the hydrosulfite adduct of a peptide aldehyde may also be written: P-A²-A¹-L-B³—B²—B¹—N(H)—CHR—CH(OH)—SO₃M, where P, A², A¹,L, B³, B², B¹, R and M are as defined above.

Alternatively, the adduct of a peptide aldehyde can be Cbz-Gly-Ala-N(H)—CH(CH₂-p-C₆H₄OH)—CH(OH)—SO₃Na (Sodium (2S)—[(N—{N-[(benzyloxy)carbonyl]glycyl}-L-alaninyl)amino]-1-hydroxy-3-(4-hydroxyphenyl)propane-1-sulfonate) or Cbz-Gly-Ala-N(H)—CH(CH2Ph)-CH(OH)—SO₃Na (Sodium (2S)—[(N—{N-[(benzyloxy)carbonyl]glycyl}-L-alaninyl)amino]-1-hydroxy-3-(phenyl)propane-1-sulfonate) or “MeO-CO_Val-Ala-N(H)—CH(CH2CH(CH₃)₂)—CH(OH)—SO₃Na (Sodium (2S)—[(N—{N-[(benzyloxy)carbonyl]glycyl}-L-alaninyl)amino]-1-hydroxy-3-(2-propanyl)propane-1-sulfonate).

Other preferred peptide aldehyde bisulfites are

Cbz-Arg-Ala-NHCH(CH₂C₆H₄OH)C(OH)(SO₃M)-H where M=Na,

Ac-Gly-Ala-NHCH(CH₂C₆H₄OH)C(OH)(SO₃M)-H, where M=Na,

Cbz-Gly-Ala-NHCH(CH₂C₆H₄OH)C(OH)(SO₃M)-H, where M=Na (L-Alaninamide, N-[(phenylmethoxy)carbonyl]glycyl-N-[2-hydroxy-1-[(4-hydroxyphenyl)methyl]-2-sulfoethyl]-, sodium salt (1:1)),

Cbz-Gly-Ala-NHCH(CH₂CH(CH₃)₂))C(OH)(SO₃M)-H, where M=Na,

Cbz-Val-Ala-NHCH(CH₂CH(CH₃)₂))C(OH)(SO₃M)-H, where M=Na,

Cbz-Gly-Ala-NHCH(CH₂Ph)C(OH)(SO₃M)-H, where M=Na,

Cbz-Gly-Ala-NHCH(CH(CH₃)₂)C(OH)(SO₃M)-H, where M=Na,

Cbz-Gly-Gly-NHCH(CH₂C₆H₄OH)C(OH)(SO₃M)-H, where M=Na,

Cbz-Gly-Gly-NHCH(CH₂Ph)C(OH)(SO₃M)-H, where M=Na,

Cbz-Arg-Val-NHCH(CH₂C₆H₄OH)C(OH)(SO₃M)-H, where M=Na,

Cbz-Leu-Val-NHCH(CH₂C₆H₄OH)C(OH)(SO₃M)-H, where M=Na,

Ac-Leu-Gly-Ala-NHCH(CH₂C₆H₄OH)C(OH)(SO₃M)-H, where M=Na,

Ac-Phe-Gly-Ala-NHCH(CH₂C₆H₄OH)C(OH)(SO₃M)-H, where M=Na,

Ac-Tyr-Gly-Ala-NHCH(CH₂C₆H₄OH)C(OH)(SO₃M)-H, where M=Na,

Ac-Phe-Gly-Ala-NHCH(CH₂CH(CH₃)₂))C(OH)(SO₃M)-H, where M=Na,

Ac-Phe-Gly-Ala-NHCH(CH₂Ph)C(OH)(SO₃M)-H, where M=Na,

Ac-Phe-Gly-Val-NHCH(CH₂C₆H₄OH)C(OH)(SO₃M)-H, where M=Na,

Ac-Phe-Gly-Ala-NHCH(CH₂CH₂SCH₃)(SO₃M)-H, where M=Na,

Ac-Trp-Leu-Val-NHCH(CH₂C₆H₄OH)C(OH)(SO₃M)-H, where M=Na,

MeO—CO-Val-Ala-NHCH(CH₂CH(CH₃)₂))C(OH)(SO₃M)-H, where M=Na,

MeNCO-Val-Ala-NHCH(CH₂CH(CH₃)₂))C(OH)(SO₃M)-H, where M=Na,

MeO—CO-Phe-Gly-Ala-NHCH(CH₂CH(CH₃)₂))C(OH)(SO₃M)-H, where M=Na,

MeO—CO-Phe-Gly-Ala-NHCH(CH₂Ph)C(OH)(SO₃M)-H, where M=Na,

MeSO₂-Phe-Gly-Ala-NHCH(CH₂CH(CH₃)₂))C(OH)(SO₃M)-H, where M=Na,

MeSO₂—Val-Ala-NHCH(CH₂CH(CH₃)₂))C(OH)(SO₃M)-H, where M=Na,

PhCH₂O(OH)(O)P-Val-Ala-NHCH(CH₂CH(CH₃)₂))C(OH)(SO₃M)-H, where M=Na,

EtSO₂-Phe-Gly-Ala-NHCH(CH₂CH(CH₃)₂))C(OH)(SO₃M)-H, where M=Na,

PhCH₂SO₂—Val-Ala-NHCH(CH₂CH(CH₃)₂))C(OH)(SO₃M)-H, where M=Na,

PhCH₂O(OH)(O)P-Leu-Ala-NHCH(CH₂CH(CH₃)₂))C(OH)(SO₃M)-H, where M=Na,

PhCH₂O(OH)(O)P-Phe-Ala-NHCH(CH₂CH(CH₃)₂))C(OH)(SO₃M)-H, where M=Na,

MeO(OH)(O)P-Leu-Gly-Ala-NHCH(CH₂CH(CH₃)₂))C(OH)(SO₃M)-H, where M=Na, and

Phe-urea-Arg-Val-NHCH(CH₂C₆H₄OH)C(OH)(SO₃M)-H where M=Na.

Salt

The salt used in the bar is a salt of a monovalent cation and an organic anion. The monovalent cation may be for example Na⁺, K⁺ or NH₄⁺. The organic anion may be for example formate, acetate, citrate or lactate. Thus, a salt of a monovalent cation and an organic anion may be, for example, sodium formate, potassium formate, ammonium formate, sodium acetate, potassium acetate, ammonium acetate, sodium lactate, potassium lactate, ammonium lactate, monosodium citrate, di-sodium citrate, tri-sodium citrate, sodium potassium citrate, potassium citrate, ammonium citrate or the like. A particular embodiment is sodium formate.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water-soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids. Ref: (US2009/0011970 A1).

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Method of Producing the Composition

The present invention also relates to methods of producing the composition.

### Uses

The present invention is also directed to methods for using the compositions thereof.

### Use in Detergents.

The polypeptides of the present invention may be added to and thus become a component of a detergent composition.

The detergent composition of the present invention may be formulated, for example, as a hand or machine laundry detergent composition including a laundry additive composition suitable for pre-treatment of stained fabrics or for rejuvenating textile (e.g. by fuzz or pill removal) to restore some of the visual and feel properties of fabrics after extended use to match that of a new textile, and a rinse added fabric softener composition, or be formulated as a detergent composition for use in general household hard surface cleaning operations, or be formulated for hand or machine dishwashing operations.

In a specific aspect, the present invention provides a detergent additive comprising a polypeptide of the present invention as described herein.

### EXAMPLES

Materials and Methods

General methods of PCR, cloning, ligation nucleotides etc. are well-known to a person skilled in the art and may for example be found in in “Molecular cloning: A laboratory manual”, Sambrook et al. (1989), Cold Spring Harbor lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “A Practical Guide To Molecular Cloning”, B. Perbal, (1984).

Assay for Cellulolytic Activity

Cellulolytic activity is determined using the Cellulase Assay Kit (CellG5 Method) provided from Megazyme, (Wicklow, Ireland; Product-code: K-CellG5-4V), following the manufacturer's instructions. The CellG5 assay reagent for the measurement of endo-cellulase (endo-1,4-β-glucanase) contains two components;

1) 4,6-O-(3-Ketobutylidene)-4-nitrophenyl-β-D-cellopentaoside (BPNPG5) and 2) thermostable β-glucosidase. The ketone blocking group prevents any hydrolytic action by the β-glucosidase on BPNPG5. Incubation with an endo-cellulase generates a non-blocked colorimetric oligosaccharide that is rapidly hydrolysed by the ancillary β-glucosidase. The rate of formation of 4-nitrophenol is therefore directly related to the hydrolysis of BPNPG5 by the endo-cellulase.

Composition of Model Detergent a (Liquid)

Composition of detergent A (liquid): Ingredients: 12% LAS, 11% AEO Biosoft N25-7 (NI), 7% AEOS (SLES), 6% MPG (monopropylene glycol), 3% ethanol, 3% TEA, 2.75% cocoa soap, 2.75% soya soap, 2% glycerol, 2% sodium hydroxide, 2% sodium citrate, 1% sodium formiate, 0.2% DTMPA and 0.2% PCA, water to 100% (all percentages are w/w).

Composition of Model Detergent A2 (Liquid)

Composition of detergent A2 (liquid): Ingredients: 12% LAS, 12% AEO Biosoft N25-7 (NI), 4% AEOS (SLES), 2% MPG (monopropylene glycol), 3% ethanol, 2% TEA (triethyl amine), 3% soap, 0.5% sodium hydroxide, 3.9% sodium citrate, 1.5% DTMPA, Na7 (diethylenetraminepentakis (methylene)pentakis(phosphonic acid), heptasodium salt), 0.5% phenoxyethanol, water to 100% (all percentages are w/w).

Protease

The proteases used for the examples is of SEQ ID NO: 10. Other proteases include those of SEQ ID NO: 11, or SEQ ID NO: 11 having mutations S9E+N42R+N74D+V1991+Q200L+Y203W+S253D+N255W+L256E.

### Wash Assays

### Launder-O-Meter (LOM) Model Wash System

The Launder-O-Meter (LOM) is a medium scale model wash system that can be applied to test up to 20 different wash conditions simultaneously. A LOM is basically a large temperature-controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time-consuming full-scale experiments in front loader washing machines.

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM is basically a large temperature-controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time-consuming full-scale experiments in front loader washing machines.

In miniLOM, washes are performed in 50 ml test tubes placed in Stuart rotator.

### Terg-O-Tometer (TOM) Wash Assay

The Terg-O-tometer (TOM) is a medium scale model wash system that can be applied to test 12 different wash conditions simultaneously. A TOM is basically a large temperature-controlled water bath with up to 12 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

The TOM model wash system is mainly used in medium scale testing of detergents and enzymes at US or LA/AP wash conditions. In a TOM experiment, factors such as the ballast to soil ratio and the fabric to wash liquor ratio can be varied. Therefore, the TOM provides the link between small scale experiments and the more time-consuming full-scale experiments in top loader washing machines.

### Example 1: Determining the Stability of Cellulase Variants (Core Stability Method)

The stability of cellulase variants is measured in 90% liquid detergent A containing protease. The in-detergent stability is assessed by measuring the activity of the variants by the CellG5 kit after incubation of the enzyme-detergent mixture containing protease.

Temperature/Protease Stress Conditions in 90% Detergent A:

In a 96well microplate (polystyrene), 20 μL of a 1000 ppm purified endo-cellulase diluted in buffer (100 mM HEPES; 0.01% Tween-20; pH 7.5)) is mixed with 180 μL of detergent A containing 0.3 mg/mL active enzyme protease protein

15 μL of the enzyme/detergent mix is transferred into two new 384 well microplates and sealed. One of the two identical plates was stored at 5° C. (reference) while the other was incubated at elevated temperature (stress) for 16 or 17 hours. See result-tables for stress-temperature used. After incubation, 60 μL of assay buffer (100 mM HEPES; 0.01% Tween-20; pH 7.5) is added to the samples in both plates and mixed vigorously for the subsequent activity measurement.

Assaying Samples for Cellulolytic Activity (CellG5 Kit):

The enzymatic activity is measured by mixing 20 μL of the diluted enzyme-detergent mixture with 10 μL assay buffer (100 mM HEPES; 0.01% Tween-20; pH 7.5) and 10 μL freshly prepared substrate solution in a UV-transparent 384well microplate. Substrate solution of the CellG5 assay kit is prepared by mixing 10 μL of bottle #2 with 300 μL bottle #1.

The UV-absorbance (405 nm) is measured kinetically (every 2nd minute for 44 minutes) using a microplate reader (Tecan; Infinite, M1000, pro). The part of the curve displaying a constant absorbance increase was used to calculate the enzymatic activity of the sample (mOD/min). Thereafter the residual activity is calculated as the enzymatic activity of the sample incubated at elevated temperature for 16 or 17 hours relative to the enzymatic activity in the corresponding sample stored at 5° C.

Residual activity (%)=(Activity,sample incubated at elevated temperature/Activity,sample incubated at 5° C.)*100

### Example 2: Linker Stability Assay

Principle

The linker stability is measured by (A) incubating the cellulase in detergent containing protease, then (B) determining the ability of the incubated cellulase to bind to cellulose fibers. If the linker or the cellulose binding domain is affected by the protease the binding affinity of the cellulase to cellulose fibers will be reduced.

The binding is determined by adding a dilution of the incubated cellulase to a suspension of cellulose fibers. After incubation at 5° C., the cellulase bound to cellulose is removed by centrifugation, and the amount of cellulase not bound to the cellulose is determined by measuring (C) the activity of cellulase in the supernatant. The activity of the cellulase not bound to the cellulose relative to the activity of a parallel sample incubated at similar conditions but in the absence of cellulose is a measure of the linker stability.

The activity is based on hydrolysis of the soluble carboxymethyl cellulose (CMC) followed by (D) detection of the number of reducing ends formed. CMC is a substrate both for the intact cellulase and cellulases having no cellulose binding domain.

A. Incubation in Detergent Containing Protease

Chemicals
Detergent: Model Detergent A
Protease: SEQ ID NO: 10
HEPES: 4-(2-hydroxyethyl)-1-piperazineethanesulfonic acid, Sigma H3375

Reagents
Dilution Buffer: 50 mM HEPES, pH 8
Protease stock solution, e.g. protease of SEQ ID NO: 10
Detergent with 0.3 mg/mL Protease: 300 ppm in Model Detergent A
Model Detergent A as above

Procedure
1) Detergent with 0.3 mg/mL protease is prepared by adding Protease stock solution to 100 mL Detergent to a final protease concentration of 300 ppm active protease protein in the detergent and mix for 1 hour by magnetic stirring at room temperature.
2) The cellulases are diluted to 300 ppm in Dilution Buffer.
3) 270 μL of Detergent with Protease from (1) is pipetted into 96-well polypropylene microplates (Thermo Scientific™ 249944) in well positions A1 to D12.
4) 30 μL of diluted cellulase from (2) is added to each well (positions A1 to D12). Each cellulase is tested in triplicates and positions D4 to D6 are used for blanks, where 30 μL Milli Q water is added instead of cellulase.
5) Small magnets are added to each well (position A1 to D12) and the plate is sealed with heat seal (Thermo Scientific™ Adhesive PCR Plate Seals AB0558) followed by mixing by magnetic stirring for 30 minutes.
6) After mixing, the plate is incubated at the time and temperature indicated in the examples.

B. Binding to Cellulose Fibers

Chemicals
Cellulose fibers: Avicel®, PH-101, (Sigma 11365)
HEPES: 4-(2-hydroxyethyl)-1-piperazineethanesulfonic acid, Sigma H3375

Reagents:
Binding Buffer: 50 mM HEPES, pH 8
Avicel suspension: 1.25 g/100 mL Avicel in Binding Buffer, mixed 1 hour before use

Procedure:
1) 180 μL Avicel suspension is added to position A1->D12 in new 96-well microplates (Thermo Scientific™ cat. No. 269620) and 180 μL Binding Buffer was added to position E1->H12
2) 20 μL sample aliquots from each well in the incubated plate from step A is then added to the wells in position A1->D12 and in position E1->H12, respectively.
3) The plate is shaken at a speed sufficient for keeping the cellulose fibers in suspension for 1 hour at 5° C. to allow the cellulase to bind to cellulose
4) After binding, the plate is centrifuged for 10 sec. at 1500 rpm and the supernatant diluted 2.5-fold in Binding Buffer (40 μL sample+60 μL buffer). Both supernatants from Avicel and corresponding wells without Avicel are diluted.

C. CMC Activity Assay

Chemicals:

CMC: Sodium carboxymethyl cellulose (Sigma C5678)

K—Na-Tartrate: Merck 8087

β-glucosidase Megazyme (Thermotoga maritima; accession number Q08638, Megazyme Cat. No. E-BGOSTM) diluted to 0.1 mg/mL (specific activity 70 U/mg and activity in product ˜460 U/mL->6.57 mg/mL)

PAHBAH 4-Hydroxybenzhydrazide (Sigma H9882)

NaOH sodium hydroxide (J.T. Baker 0402.1000)

HEPES: 4-(2-hydroxyethyl)-1-piperazineethanesulfonic acid, Sigma H3375

Reagents:

Assay Buffer: 50 mM HEPES, pH 8

CMC Substrate: 1.25 g CMC/100 mL Assay Buffer, mixed 1 hr before use

PAHBAH Buffer: 50 g/L K—Na-tartrate+20 g/L NaOH

PAHBAH Reagent: 15 mg/mL PAHBAH in PAHBAH Buffer

β-glucosidase Solution: 0.1 mg/mL β-glucosidase in Assay Buffer

Procedure:
1) 160 μL CMC Substrate is pipetted into new 96-well microplates (Thermo Scientific™ cat. No. 269620)
2) 20 μL diluted supernatant from step B is added together with 20 μL β-glucosidase Solution
3) The plate was sealed with heat seal (Thermo Scientific™ Adhesive PCR Plate Seals AB0558), and incubated for 45 minutes at 40° C.
4) After reaction, 100 μL from each well is transferred to ThermoFast 96 Non-Skirted® PCR plate (Thermo Scientific™ cat. No. AB-0600) followed by 75 μL PAHBAH Reagent
5) The plate from (4) is then sealed with sealing foil (Greiner bio-one platesealer, Cat. No. 676001) and incubated at 95° C. for 10 minutes followed by cooling at 10° C. for 5 minutes in BioRad T100™ Thermal cycler PCR machine
6) After cooling, 100 μL aliquots are transferred to new 96-well microplates (Thermo Scientific™ cat. No. 269620) and the absorbance is read at 405 nm (A_{405 nm}). The absorbance is an expression of the activity of the cellulase in the supernatant.

D. Data-Treatment
1) From the absorbance readings from step C, the average of the 3 blanks from the wells without Avicel, A_{405 nm}(blank ref), is calculated (position H4->H6), and the average of the 3 blanks from the wells with Avicel, A_{405 nm}(blank Avicel) is calculated (position D4->D6).
2) The absorbance readings from the cellulase containing wells are then corrected for their respective blanks (i.e. those from (1)).
3) The linker stability is calculated as

1−[Act_{405 nm}(+Avicel)/Act_{405 nm}(−Avicel)],
where Act_{405 nm}(+Avicel) and Act_{405 nm}(−Avicel) is the activity (i.e. absorbance corrected for blank) in the well with supernatant from incubation with Avicel and without Avicel, respectively.
4) The linker stabilities reported in the examples are the averages of the triplicates analyzed.

This assay clearly distinguishes over binding with and without the core present, as further demonstrated by Example 3 below.

### Example 3: Cellulose Binding Assay—without Protease

### Principle

The cellulase is (A) allowed to bind to cellulose by incubation with Avicel at 5° C. for 60 minutes in a dilute detergent solution. After incubation, the activity of the cellulase not bound to the cellulose is determined in the supernatant (B) and compared relative to a parallel cellulase sample incubated in the absence of cellulase. The temperature during incubation with Avicel is kept low to ensure that the catalytic activity of the cellulase during the binding step has an insignificant effect on the binding assay.

A. Binding to Cellulose

Chemicals

Detergent: Model Detergent A

HEPES: 4-(2-hydroxyethyl)-1-piperazineethanesulfonic acid, Sigma H3375

Cellulose fibers: Avicel®, PH-101, (Sigma 11365)

Reagents

Binding Buffer: 50 mM HEPES, pH 8

Avicel Suspension: 1.25 g/100 mL Avicel in Binding Buffer, mixed 1 hour before use

Model Detergent A as above

Procedure
1) The cellulases are diluted to 300 ppm in Binding Buffer.
2) 270 μL of Detergent is pipetted into 96-well polypropylene microplates (Thermo Scientific™ 249944) in well positions A1 to D12.
3) 30 μL of diluted cellulase from (1) is added to each well (positions A1 to D12). Each cellulase is tested in triplicates and positions D4 to D6 are used for blanks, where 30 μL Milli Q water is added instead of cellulase.
4) Small magnets are added to each well (position A1 to D12) and the plate is sealed with heat seal (Thermo Scientific Adhesive PCR Plate Seals AB0558) followed by mixing by magnetic stirring for 30 minutes.
5) 160 μL Binding buffer is pipetted into new 96-well microplate (Thermo Scientific™ Nunc™ 96-well Polypropylene DeepWell™ Storage Plates (position A1 to D12 and 160 μL Avicel suspension is pipetted into position E1 to H12.
6) 20 μL Milli Q water is added to all wells (A1 to H12)
7) Aliquots of 20 μL cellulase-detergent sample from ((4) is added to wells with (A1 to D12) and without Avicel (E1 to H12).
8) The plate is then incubated for 60 minutes in 5° C. cold room to allow binding of the cellulase to the cellulose in a Heidolph Titramax 101 shaker. Shaking speed is adjusted to ensure cellulose is kept suspended during incubation.
9) After binding, the plate is centrifuged for 10 sec. at 1500 rpm and the supernatant diluted 2.5-fold in Binding Buffer (40 μL sample+60 μL buffer). Both supernatants from Avicel and corresponding wells without Avicel are diluted.

B. CMC Activity Assay

Chemicals:

CMC: Sodium carboxymethyl cellulose (Sigma C5678)

K—Na-Tartrate: Merck 8087

β-glucosidase Megazyme (Thermotoga maritima; accession number Q08638, Megazyme Cat. No. E-BGOSTM) diluted to 0.1 mg/mL (specific activity 70 U/mg and activity in product ˜460 U/mL->6.57 mg/mL)

PAHBAH 4-Hydroxybenzhydrazide (Sigma H9882)

NaOH sodium hydroxide (J.T. Baker 0402.1000)

HEPES: 4-(2-hydroxyethyl)-1-piperazineethanesulfonic acid, Sigma H3375

Reagents:

Assay Buffer: 50 mM HEPES, pH 8

CMC Substrate: 1.25 g CMC/100 mL Assay Buffer, mixed 1 hr before use

PAHBAH Buffer: 50 g/L K—Na-tartrate+20 g/L NaOH

PAHBAH Reagent: 15 mg/mL PAHBAH in PAHBAH Buffer

β-glucosidase Solution: 0.1 mg/mL β-glucosidase in Assay Buffer

Procedure:
1) 160 μL CMC Substrate is pipetted into new 96-well microplates (Thermo Scientific cat. No. 269620)
2) 20 μL diluted supernatant from step A is added together with 20 μL β-glucosidase Solution 3) The plate was sealed with heat seal (Thermo Scientific Adhesive PCR Plate Seals AB0558), and incubated for 45 minutes at 40° C.
4) After reaction, 100 μL from each well is transferred to ThermoFast 96 Non-Skirted® PCR plate (Thermo Scientific cat. No. AB-0600) followed by 75 μL PAHBAH Reagent
5) The plate from (4) is then sealed with sealing foil (Greiner bio-one platesealer, Cat. No. 676001) and incubated at 95° C. for 10 minutes followed by cooling at 10° C. for 5 minutes in BioRad T100™ Thermal cycler PCR machine
6) After cooling, 100 μL aliquots are transferred to new 96-well microplates (Thermo Scientific cat. No. 269620) and the absorbance is read at 405 nm (A_{405 nm}). The absorbance is an expression of the activity of the cellulase in the supernatant.

C. Data-Treatment
1) From the absorbance readings from step B, the average of the 3 blanks from the wells without Avicel, A_{405 nm}(blank ref), is calculated (position H4->H6), and the average of the 3 blanks from the wells with Avicel, A_{405 nm}(blank Avicel) is calculated (position D4->D6).
2) The absorbance readings from the cellulase containing wells are then corrected for their respective blanks (i.e. those from (1)).
3) The binding is calculated as

1−[Act_{405 nm}(+Avicel)/Act_{405 nm}(−Avicel)],

where Act_{405 nm}(+Avicel) and Act_{405 nm}(−Avicel) is the activity (i.e. absorbance corrected for blank) in the well with supernatant from incubation with Avicel and without Avicel, respectively. The linker stabilities reported in the examples are the averages of the triplicates analyzed.

To demonstrate this, samples of cellulases with and without CBM were tested for binding to cellulose as described in this Example. The ratio is calculated as the binding of the intact cellulase, i.e. cellulase with catalytic domain, linker and CRM to that of the catalytic domain alone.

| Variant | Binding | Ratio |
|---|---|---|
| SEQ ID NO: 2 | 0.79 | 4.6 |
| Amino acids 1-213 of SEQ ID | 0.17 | |
| NO: 2* | | |
| SEQ ID NO: 1 | 0.58 | 5.2 |
| Amino acids 1-216 of SEQ ID | 0.11 | |
| NO: 1* | | |

*Sequence length reflects catalytic domain as annotated by bioinformatics processing

The data clearly demonstrates that in the absence of the CBM, the binding to cellulose is significantly reduced.

### Example 4: Construction of Glycoside Hydrolase Variants

Cellulase variants are constructed of the Thielavia terrestris cellulase (SEQ ID NO: 1). The variants were made by traditional cloning of DNA fragments (Sambrook et al., Molecular Cloning: A Laboratory Manual, 2nd Ed., Cold Spring Harbor, 1989) using PCR together with properly designed oligonucleotides that introduced the desired mutations in the resulting sequence. Alternatively, synthetic gene fragments purchased from vendors such IDTDNA were used to replace the native DNA sequence with the new, desired DNA sequence.

The oligos are designed corresponding to the DNA sequence flanking the desired site(s) of mutation or stretch of DNA to be replaced, separated by the DNA base pairs defining the insertions/deletions/substitution/synthetic DNA sequence, and purchased from an oligo vendor such as IDTDNA. In order to test the variants of the invention, the mutated DNA comprising a variant of the invention are integrated into a competent A. oryzae strain by homologous recombination, fermented using standard protocols (yeast extract based media, 4-5 days, 30° C.), and purified as follows.

Culture broth is filtered through a Nalgene 0.2 μm filtration unit to remove the Aspergillus host cells. The pH in the filtrate is adjusted to pH 4.0 with 20% CH₃COOH and the pH adjusted filtrate was applied to a Capto MMC column (from GE Healthcare) equilibrated in 20 mM CH₃COOH/NaOH, 1 mM CaCl₂, pH 4.0. After washing the column extensively with the equilibration buffer, the cellulase is eluted with 50 mM Tris-base, 1 mM CaCl₂, unbuffered. Fractions from the column are analysed for cellulase activity. The cellulase peak is pooled and applied to a Q-sepharose FF column (from GE Healthcare) equilibrated in 50 mM Tris/HCl, pH 9.0. After washing the column extensively with the equilibration buffer, the cellulase is eluted with a linear NaCl gradient over three column volumes between the equilibration buffer and 50 mM Tris/HCl, 5 mM CaCl₂, 500 mM NaCl, pH 9.0. Fractions from the column are analysed for cellulase activity and the cellulase peak is pooled as the purified product. The purified variants are analysed by SDS-PAGE. As the cellulase variants are glycosylated they gave diffuse bands on coomassie stained SDS-PAGE gels. The purified products are used for further characterization.

### Example 5: In-Wash Linker Stability Assay with Proteases

Principle

The linker stability is measured by (A) incubating the cellulase in detergent wash-solution containing protease, then (B) determining the ability of the incubated cellulase to bind to cellulose fibers. If the linker or the cellulose binding domain is affected by the protease the binding affinity of the cellulase to cellulose fibers will be reduced.

The binding is determined by adding a dilution of the incubated cellulase to a suspension of cellulose fibers. After incubation at 5° C., the cellulase bound to cellulose is removed by centrifugation, and the amount of cellulase not bound to the cellulose is determined by measuring (C) the activity of cellulase in the supernatant. The activity of the cellulase not bound to the cellulose relative to the activity of a parallel sample incubated at similar conditions but in the absence of cellulose is a measure of the linker stability.

The activity is based on hydrolysis of the soluble carboxymethyl cellulose (CMC) followed by (D) detection of the number of reducing ends formed. CMC is a substrate both for the intact cellulase and cellulases having no cellulose binding domain.

E. Incubation in Detergent Containing Protease

Chemicals

Detergent: Model Detergent A

Protease: Protease with SEQ ID NO: 10

HEPES: 4-(2-hydroxyethyl)-1-piperazineethanesulfonic acid, Sigma H3375

Reagents

Dilution Buffer: 50 mM HEPES, pH 8

Detergent with Protease: 0.3 μg/mL active protease protein in Model Detergent A

Detergent wash-solution: 3.3 g/L Detergent with Protease in water with 15° dH water hardness.

Procedure
7) Detergent wash-solution is prepared
8) The cellulases are diluted to 300 ppm in Dilution Buffer.
9) 270 μL of Detergent wash-solution from (1) is pipetted into 96-well polypropylene microplates (Thermo Scientific™ 249944) in well positions A1 to D12.
10) 30 μL of diluted cellulase from (2) is added to each well (positions A1 to D12). Each cellulase is tested in triplicates and positions D4 to D6 are used for blanks, where 30 μL Milli Q water is added instead of cellulase.
11) Small magnets are added to each well (position A1 to D12) and the plate is sealed with heat seal (Thermo Scientific™ Adhesive PCR Plate Seals AB0558) followed by mixing by magnetic stirring for 30 minutes.
12) After mixing, the plate is incubated at the time and temperature indicated in the examples, e.g. 2 hours at 40° C.

F. Binding to Cellulose Fibers

Chemicals

Cellulose fibers: Avicel®, PH-101, (Sigma 11365)

HEPES: 4-(2-hydroxyethyl)-1-piperazineethanesulfonic acid, Sigma H3375

Reagents:

Binding Buffer: 50 mM HEPES, pH 8

Avicel suspension: 1.25 g/100 mL Avicel in Binding Buffer, mixed 1 hour before use

Procedure:
5) 180 μL Avicel suspension is added to position A1->D12 in new 96-well microplates (Thermo Scientific™ cat. No. 269620) and 180 μL Binding Buffer was added to position E1->H12
6) 20 μL sample aliquots from each well in the incubated plate from step A is then added to the wells in position A1->D12 and in position E1->H12, respectively.
7) The plate is shaken at a speed sufficient for keeping the cellulose fibers in suspension for 1 hour at 5° C. to allow the cellulase to bind to cellulose
8) After binding, the plate is centrifuged for 10 sec. at 1500 rpm and the supernatant diluted 2.5-fold in Binding Buffer (40 μL sample+60 μL buffer). Both supernatants from Avicel and corresponding wells without Avicel are diluted.

G. CMC Activity Assay

Chemicals:

CMC: Sodium carboxymethyl cellulose (Sigma C5678)

K—Na-Tartrate: Merck 8087

β-glucosidase Megazyme (Thermotoga maritima; accession number Q08638, Megazyme Cat. No. E-BGOSTM) diluted to 0.1 mg/mL (specific activity 70 U/mg and activity in product ˜460 U/mL->6.57 mg/mL)

PAHBAH 4-Hydroxybenzhydrazide (Sigma H9882)

NaOH sodium hydroxide (J.T. Baker 0402.1000)

HEPES: 4-(2-hydroxyethyl)-1-piperazineethanesulfonic acid, Sigma H3375

Reagents:

Assay Buffer: 50 mM HEPES, pH 8

CMC Substrate: 1.25 g CMC/100 mL Assay Buffer, mixed 1 hr before use

PAHBAH Buffer: 50 g/L K—Na-tartrate+20 g/L NaOH

PAHBAH Reagent: 15 mg/mL PAHBAH in PAHBAH Buffer

β-glucosidase Solution: 0.1 mg/mL β-glucosidase in Assay Buffer

Procedure:
7) 160 μL CMC Substrate is pipetted into new 96-well microplates (Thermo Scientific™ cat. No. 269620)
8) 20 μL diluted supernatant from step B is added together with 20 μL β-glucosidase Solution
9) The plate was sealed with heat seal (Thermo Scientific™ Adhesive PCR Plate Seals AB0558), and incubated for 45 minutes at 40° C.
10) After reaction, 100 μL from each well is transferred to ThermoFast 96 Non-Skirted® PCR plate (Thermo Scientific™ cat. No. AB-0600) followed by 75 μL PAHBAH Reagent
11) The plate from (4) is then sealed with sealing foil (Greiner bio-one platesealer, Cat. No. 676001) and incubated at 95° C. for 10 minutes followed by cooling at 10° C. for 5 minutes in BioRad T100™ Thermal cycler PCR machine
12) After cooling, 100 μL aliquots are transferred to new 96-well microplates (Thermo Scientific™ cat. No. 269620) and the absorbance is read at 405 nm (A_{405 nm}). The absorbance is an expression of the activity of the cellulase in the supernatant.

H. Data-Treatment
5) From the absorbance readings from step C, the average of the 3 blanks from the wells without Avicel, A_{405 nm}(blank ref), is calculated (position H4->H6), and the average of the 3 blanks from the wells with Avicel, A_{405 nm}(blank Avicel) is calculated (position D4->D6).
6) The absorbance readings from the cellulase containing wells are then corrected for their respective blanks (i.e. those from (1)).
7) The linker stability is calculated as

1−[Act_{405 nm}(+Avicel)/Act_{405 nm}(−Avicel)],
where Act_{405 nm}(+Avicel) and Act_{405 nm}(−Avicel) is the activity (i.e. absorbance corrected for blank) in the well with supernatant from incubation with Avicel and without Avicel, respectively.
8) The linker stabilities reported in the examples are the averages of the triplicates analyzed.

### Example 6: Variants of CBM SEQ ID NO:173 with P7G Linker (SEQ ID NO:30)

Variants were tested as described in the example 2, Linker Stability Assay, with the following modifications: the detergent was Model Detergent A2, the protease SEQ ID NO. 10. The variants were incubated at 45° C. for 3 days in the detergent with the protease before being tested for residual activity and ability to bind to cellulose.

| | | CBM | | Relative |
|---|---|---|---|---|
| Example | Catalytic domain | (SEQ ID NO | Linker | linker |
| ID | (SEQ ID NO) | and mutation) | (SEQ ID NO) | stability |
| 1 | SEQ ID NO: 5 | SEQ ID NO: 7 | SEQ ID NO: 30 | 1.0 |
| | having mutations | | | |
| | A32S S56A | | | |
| | N134D A146D | | | |
| | Q147R Q169Y | | | |
| | F183V | | | |
| 2 | SEQ ID NO: 5 | SEQ ID NO: 173 | SEQ ID NO: 30 | 3.9 |
| | having mutations | | | |
| | A32S S56A | | | |
| | N134D A146D | | | |
| | Q147R Q169Y | | | |
| | F183V | | | |
| 3 | SEQ ID NO: 5 | SEQ ID NO: 173 + | SEQ ID NO: 30 | 4.8 |
| | having mutations | S34H | | |
| | A32S S56A | | | |
| | N134D A146D | | | |
| | Q147R Q169Y | | | |
| | F183V | | | |
| 4 | SEQ ID NO: 5 | SEQ ID NO: 173 + | SEQ ID NO: 30 | 3.4 |
| | having mutations | S34Y | | |
| | A32S S56A | | | |
| | N134D A146D | | | |
| | Q147R Q169Y | | | |
| | F183V | | | |
| 5 | SEQ ID NO: 5 | SEQ ID NO: 173 + | SEQ ID NO: 30 | 1.1 |
| | having mutations | S34K | | |
| | A32S S56A | | | |
| | N134D A146D | | | |
| | Q147R Q169Y | | | |
| | F183V | | | |
| 6 | SEQ ID NO: 5 | SEQ ID NO: 173 + | SEQ ID NO: 30 | 3.2 |
| | having mutations | L37R | | |
| | A32S S56A | | | |
| | N134D A146D | | | |
| | Q147R Q169Y | | | |
| | F183V | | | |
| 7 | SEQ ID NO: 5 | SEQ ID NO: 173 + | SEQ ID NO: 30 | 3.5 |
| | having mutations | S34Y+ L37R | | |
| | A32S S56A | | | |
| | N134D A146D | | | |
| | Q147R Q169Y | | | |
| | F183V | | | |
| 8 | SEQ ID NO: 5 | SEQ ID NO: 173 + | SEQ ID NO: 30 | 3.1 |
| | having mutations | S34H + L37R | | |
| | A32S S56A | | | |
| | N134D A146D | | | |
| | Q147R Q169Y | | | |
| | F183V | | | |
| 9 | SEQ ID NO: 5 | SEQ ID NO: 173 + | SEQ ID NO: 30 | 3.7 |
| | having mutations | A22E | | |
| | A32S S56A | | | |
| | N134D A146D | | | |
| | Q147R Q169Y | | | |
| | F183V | | | |
| 10 | SEQ ID NO: 5 | SEQ ID NO: 173 + | SEQ ID NO: 30 | 7.8 |
| | having mutations | A22N | | |
| | A32S S56A | | | |
| | N134D A146D | | | |
| | Q147R Q169Y | | | |
| | F183V | | | |
| 11 | SEQ ID NO: 5 | SEQ ID NO: 173 + | SEQ ID NO: 30 | 2.3 |
| | having mutations | A22P | | |
| | A32S S56A | | | |
| | N134D A146D | | | |
| | Q147R Q169Y | | | |
| | F183V | | | |
| 12 | SEQ ID NO: 5 | SEQ ID NO: 173 + | SEQ ID NO: 30 | 3.3 |
| | having mutations | L29P | | |
| | A32S S56A | | | |
| | N134D A146D | | | |
| | Q147R Q169Y | | | |
| | F183V | | | |
| 13 | SEQ ID NO: 5 | SEQ ID NO: 173 + | SEQ ID NO: 30 | 8.5 |
| | having mutations | Q28P | | |
| | A32S S56A | | | |
| | N134D A146D | | | |
| | Q147R Q169Y | | | |
| | F183V | | | |
| 14 | SEQ ID NO: 5 | SEQ ID NO: 173 + | SEQ ID NO: 30 | 2.1 |
| | having mutations | T18R | | |
| | A32S S56A | | | |
| | N134D A146D | | | |
| | Q147R Q169Y | | | |
| | F183V | | | |
| 15 | SEQ ID NO: 5 | SEQ ID NO: 173 + | SEQ ID NO: 30 | 8.8 |
| | having mutations | T27I | | |
| | A32S S56A | | | |
| | N134D A146D | | | |
| | Q147R Q169Y | | | |
| | F183V | | | |

Notation with respect to CEM: SEQ ID NO followed by “+” indicates that the specified mutation has been introduced in the referenced sequence. E.g. 173+S34H indicates that S in position 34 of SEQ ID NO: 173 has been substituted with H.

The data clearly demonstrates the positive effect of substituting the OEM having SEQ ID NO:7 with the OEM having SEQ ID NO:173 or variants of OEM having SEQ ID NO:173.

### Example 7: Variants of CBM SEQ ID NO:173 with Various Linkers

Variants of SEQ ID NO:173 with different linkers were tested as described in the example 2, Linker Stability Assay, with the following modifications: the detergent was Model Detergent A2, the protease SEQ ID NO. 10. The variants were incubated at 45° C. for 3 days in the detergent with the protease before being tested for residual activity and ability to bind to cellulose.

| | | CBM | | Relative |
|---|---|---|---|---|
| | Catalytic domain | (SEQ ID NO | Linker | linker |
| Example ID | (SEQ ID NO) | and mutation) | (SEQ ID NO) | stability |
| 1 | SEQ ID NO: 5 | SEQ ID NO: 7 | SEQ ID NO: 30 | 1.0 |
| | having mutations | | | |
| | A32S S56A | | | |
| | N134D A146D | | | |
| | Q147R Q169Y | | | |
| | F183V | | | |
| 16 | SEQ ID NO: 5 | SEQ ID NO: 173 + | SEQ ID NO: 95 | 1.2 |
| | having mutations | S34H + L37R | | |
| | A32S S56A | | | |
| | N134D A146D | | | |
| | Q147R Q169Y | | | |
| | F183V | | | |
| 17 | SEQ ID NO: 5 | SEQ ID NO: 173 + | SEQ ID NO: 37 | 3.1 |
| | having mutations | S34H + L37R | | |
| | A32S S56A | | | |
| | N134D A146D | | | |
| | Q147R Q169Y | | | |
| | F183V | | | |
| 18 | SEQ ID NO: 5 | SEQ ID NO: 173 + | SEQ ID NO: 85 | 4.3 |
| | having mutations | S34H + L37R | | |
| | A32S S56A | | | |
| | N134D A146D | | | |
| | Q147R Q169Y | | | |
| | F183V | | | |
| 19 | SEQ ID NO: 5 | SEQ ID NO: 173 + | SEQ ID NO: 89 | 4.4 |
| | having mutations | S34H + L37R | | |
| | A32S S56A | | | |
| | N134D A146D | | | |
| | Q147R Q169Y | | | |
| | F183V | | | |
| 20 | SEQ ID NO: 5 | SEQ ID NO: 173 + | SEQ ID NO: 96 | 2.1 |
| | having mutations | S34H, + L37R | | |
| | A32S S56A | | | |
| | N134D A146D | | | |
| | Q147R Q169Y | | | |
| | F183V | | | |
| 23 | SEQ ID NO: 5 | SEQ ID NO: 173 + | SEQ ID NO: 16 | 1.3 |
| | having mutations | S34H + L37R | | |
| | A32S S56A | | | |
| | N134D A146D | | | |
| | Q147R Q169Y | | | |
| | F183V | | | |
| 24 | SEQ ID NO: 5 | SEQ ID NO: 173 + | SEQ ID NO: 33 | 1.6 |
| | having mutations | S34H + L37R | | |
| | A32S S56A | | | |
| | N134D A146D | | | |
| | Q147R Q169Y | | | |
| | F183V | | | |
| 25 | SEQ ID NO: 5 | SEQ ID NO: 173 + | SEQ ID NO: 41 | 1.4 |
| | having mutations | S34H + L37R | | |
| | A32S S56A | | | |
| | N134D A146D | | | |
| | Q147R Q169Y | | | |
| | F183V | | | |
| 26 | SEQ ID NO: 5 | SEQ ID NO: 173 + | SEQ ID NO: 71 | 1.2 |
| | having mutations | S34H + L37R | | |
| | A32S S56A | | | |
| | N134D A146D | | | |
| | Q147R Q169Y | | | |
| | F183V | | | |
| 27 | SEQ ID NO: 5 | SEQ ID NO: 173 + | SEQ ID NO: 90 | 1.5 |
| | having mutations | S34H + L37R | | |
| | A32S S56A | | | |
| | N134D A146D | | | |
| | Q147R Q169Y | | | |
| | F183V | | | |
| 28 | SEQ ID NO: 5 | SEQ ID NO: 173 + | SEQ ID NO: 69 | 1.3 |
| | having mutations | S34H + L37R | | |
| | A32S S56A | | | |
| | N134D A146D | | | |
| | Q147R Q169Y | | | |
| | F183V | | | |
| 29 | SEQ ID NO: 5 | SEQ ID NO: 173 + | SEQ ID NO: 65 | 2.2 |
| | having mutations | S34H + L37R | | |
| | A32S S56A | | | |
| | N134D A146D | | | |
| | Q147R Q169Y | | | |
| | F183V | | | |
| 30 | SEQ ID NO: 5 | SEQ ID NO: 173 + | SEQ ID NO: 66 | 1.5 |
| | having mutations | S34H + L37R | | |
| | A32S S56A | | | |
| | N134D A146D | | | |
| | Q147R Q169Y | | | |
| | F183V | | | |
| 31 | SEQ ID NO: 5 | SEQ ID NO: 173 + | SEQ ID NO: 67 | 2.0 |
| | having mutations | S34H + L37R | | |
| | A32S S56A | | | |
| | N134D A146D | | | |
| | Q147R Q169Y | | | |
| | F183V | | | |
| 32 | SEQ ID NO: 5 | SEQ ID NO: 173 + | SEQ ID NO: 68 | 2.5 |
| | having mutations | S34H + L37R | | |
| | A32S S56A | | | |
| | N134D A146D | | | |
| | Q147R Q169Y | | | |
| | F183V | | | |
| 33 | SEQ ID NO: 5 | SEQ ID NO: 173 + | SEQ ID NO: 70 | 1.7 |
| | having mutations | S34H + L37R | | |
| | A32S S56A | | | |
| | N134D A146D | | | |
| | Q147R Q169Y | | | |
| | F183V | | | |
| 34 | SEQ ID NO: 5 | SEQ ID NO: 173 + | SEQ ID NO: 73 | 2.5 |
| | having mutations | S34H + L37R | | |
| | A32S S56A | | | |
| | N134D A146D | | | |
| | Q147R Q169Y | | | |
| | F183V | | | |
| 35 | SEQ ID NO: 5 | SEQ ID NO: 173 + | SEQ ID NO: 75 | 2.1 |
| | having mutations | S34H + L37R | | |
| | A32S S56A | | | |
| | N134D A146D | | | |
| | Q147R Q169Y | | | |
| | F183V | | | |
| 36 | SEQ ID NO: 5 | SEQ ID NO: 173 + | SEQ ID NO: 77 | 2.7 |
| | having mutations | S34H + L37R | | |
| | A32S S56A | | | |
| | N134D A146D | | | |
| | Q147R Q169Y | | | |
| | F183V | | | |
| 37 | SEQ ID NO: 5 | SEQ ID NO: 173 + | SEQ ID NO: 80 | 3.2 |
| | having mutations | S34H + L37R | | |
| | A32S S56A | | | |
| | N134D A146D | | | |
| | Q147R Q169Y | | | |
| | F183V | | | |
| 38 | SEQ ID NO: 5 | SEQ ID NO: 173 + | SEQ ID NO: 81 | 2.5 |
| | having mutations | S34H + L37R | | |
| | A32S S56A | | | |
| | N134D A146D | | | |
| | Q147R Q169Y | | | |
| | F183V | | | |
| 39 | SEQ ID NO: 5 | SEQ ID NO: 173 + | SEQ ID NO: 86 | 3.5 |
| | having mutations | S34H, L37R | | |
| | A32S S56A | | | |
| | N134D A146D | | | |
| | Q147R Q169Y | | | |
| | F183V | | | |
| 40 | SEQ ID NO: 5 | SEQ ID NO: 173 + | SEQ ID NO: 87 | 2.9 |
| | having mutations | S34H + L37R | | |
| | A32S S56A | | | |
| | N134D A146D | | | |
| | Q147R Q169Y | | | |
| | F183V | | | |
| 41 | SEQ ID NO: 5 | SEQ ID NO: 173 + | SEQ ID NO: 88 | 3.7 |
| | having mutations | S34H + L37R | | |
| | A32S S56A | | | |
| | N134D A146D | | | |
| | Q147R Q169Y | | | |
| | F183V | | | |
| 42 | SEQ ID NO: 5 | SEQ ID NO: 173 + | SEQ ID NO: 91 | 3.4 |
| | having mutations | S34H + L37R | | |
| | A32S S56A | | | |
| | N134D A146D | | | |
| | Q147R Q169Y | | | |
| | F183V | | | |
| 43 | SEQ ID NO: 5 | SEQ ID NO: 173 + | SEQ ID NO: 92 | 3.5 |
| | having mutations | S34H + L37R | | |
| | A32S S56A | | | |
| | N134D A146D | | | |
| | Q147R Q169Y | | | |
| | F183V | | | |
| 44 | SEQ ID NO: 5 | SEQ ID NO: 173 + | SEQ ID NO: 93 | 1.2 |
| | having mutations | S34H + L37R | | |
| | A32S S56A | | | |
| | N134D A146D | | | |
| | Q147R Q169Y | | | |
| | F183V | | | |
| 45 | SEQ ID NO: 5 | SEQ ID NO: 173 + | SEQ ID NO: 18 | 4.8 |
| | having mutations | S34H + L37R | | |
| | A32S S56A | | | |
| | N134D A146D | | | |
| | Q147R Q169Y | | | |
| | F183V | | | |
| 46 | SEQ ID NO: 5 | SEQ ID NO: 173 + | SEQ ID NO: 26 | 4.1 |
| | having mutations | S34H + L37R | | |
| | A32S S56A | | | |
| | N134D A146D | | | |
| | Q147R Q169Y | | | |
| | F183V | | | |
| 47 | SEQ ID NO: 5 | SEQ ID NO: 173 + | SEQ ID NO: 29 | 1.8 |
| | having mutations | S34H + L37R | | |
| | A32S S56A | | | |
| | N134D A146D | | | |
| | Q147R Q169Y | | | |
| | F183V | | | |
| 48 | SEQ ID NO: 5 | SEQ ID NO: 173 + | SEQ ID NO: 45 | 1.2 |
| | having mutations | S34H + L37R | | |
| | A32S S56A | | | |
| | N134D A146D | | | |
| | Q147R Q169Y | | | |
| | F183V | | | |
| 49 | SEQ ID NO: 5 | SEQ ID NO: 173 + | SEQ ID NO: 47 | 1.8 |
| | having mutations | S34H + L37R | | |
| | A32S S56A | | | |
| | N134D A146D | | | |
| | Q147R Q169Y | | | |
| | F183V | | | |
| 50 | SEQ ID NO: 5 | SEQ ID NO: 173 + | SEQ ID NO: 50 | 1.5 |
| | having mutations | S34H + L37R | | |
| | A32S S56A | | | |
| | N134D A146D | | | |
| | Q147R Q169Y | | | |
| | F183V | | | |
| 51 | SEQ ID NO: 5 | SEQ ID NO: 173 + | SEQ ID NO: 51 | 1.5 |
| | having mutations | S34H + L37R | | |
| | A32S S56A | | | |
| | N134D A146D | | | |
| | Q147R Q169Y | | | |
| | F183V | | | |
| 52 | SEQ ID NO: 5 | SEQ ID NO: 173 + | SEQ ID NO: 52 | 1.3 |
| | having mutations | S34H + L37R | | |
| | A32S S56A | | | |
| | N134D A146D | | | |
| | Q147R Q169Y | | | |
| | F183V | | | |
| 53 | SEQ ID NO: 5 | SEQ ID NO: 173 + | SEQ ID NO: 54 | 3.0 |
| | having mutations | S34H + L37R | | |
| | A32S S56A | | | |
| | N134D A146D | | | |
| | Q147R Q169Y | | | |
| | F183V | | | |
| 54 | SEQ ID NO: 5 | SEQ ID NO: 173 + | SEQ ID NO: 56 | 2.3 |
| | having mutations | S34H + L37R | | |
| | A32S S56A | | | |
| | N134D A146D | | | |
| | Q147R Q169Y | | | |
| | F183V | | | |
| 55 | SEQ ID NO: 5 | SEQ ID NO: 173 + | SEQ ID NO: 57 | 4.0 |
| | having mutations | S34H + L37R | | |
| | A32S S56A | | | |
| | N134D A146D | | | |
| | Q147R Q169Y | | | |
| | F183V | | | |
| 56 | SEQ ID NO: 5 | SEQ ID NO: 173 + | SEQ ID NO: 59 | 2.1 |
| | having mutations | S34H + L37R | | |
| | A32S S56A | | | |
| | N134D A146D | | | |
| | Q147R Q169Y | | | |
| | F183V | | | |
| 57 | SEQ ID NO: 5 | SEQ ID NO: 173 + | SEQ ID NO: 60 | 2.6 |
| | having mutations | S34H + L37R | | |
| | A32S S56A | | | |
| | N134D A146D | | | |
| | Q147R Q169Y | | | |
| | F183V | | | |
| 58 | SEQ ID NO: 5 | SEQ ID NO: 173 + | SEQ ID NO: 62 | 3.0 |
| | having mutations | S34H + L37R | | |
| | A32S S56A | | | |
| | N134D A146D | | | |
| | Q147R Q169Y | | | |
| | F183V | | | |
| 59 | SEQ ID NO: 5 | SEQ ID NO: 173 + | SEQ ID NO: 63 | 1.5 |
| | having mutations | S34H + L37R | | |
| | A32S S56A | | | |
| | N134D A146D | | | |
| | Q147R Q169Y | | | |
| | F183V | | | |
| 60 | SEQ ID NO: 5 | SEQ ID NO: 173 + | SEQ ID NO: 64 | 3.6 |
| | having mutations | S34H + L37R | | |
| | A32S S56A | | | |
| | N134D A146D | | | |
| | Q147R Q169Y | | | |
| | F183V | | | |
| 61 | SEQ ID NO: 5 | SEQ ID NO: 173 + | SEQ ID NO: 20 | 1.2 |
| | having mutations | S34H + L37R | | |
| | A32S S56A | | | |
| | N134D A146D | | | |
| | Q147R Q169Y | | | |
| | F183V | | | |
| 62 | SEQ ID NO: 5 | SEQ ID NO: 173 + | SEQ ID NO: 27 | 2.0 |
| | having mutations | S34H + L37R | | |
| | A32S S56A | | | |
| | N134D A146D | | | |
| | Q147R Q169Y | | | |
| | F183V | | | |
| 63 | SEQ ID NO: 5 | SEQ ID NO: 173 + | SEQ ID NO: 28 | 2.1 |
| | having mutations | S34H + L37R | | |
| | A32S S56A | | | |
| | N134D A146D | | | |
| | Q147R Q169Y | | | |
| | F183V | | | |
| 64 | SEQ ID NO: 5 | SEQ ID NO: 173 + | SEQ ID NO: 31 | 1.7 |
| | having mutations | S34H + L37R | | |
| | A32S S56A | | | |
| | N134D A146D | | | |
| | Q147R Q169Y | | | |
| | F183V | | | |
| 65 | SEQ ID NO: 5 | SEQ ID NO: 173 + | SEQ ID NO: 32 | 2.3 |
| | having mutations | S34H + L37R | | |
| | A32S S56A | | | |
| | N134D A146D | | | |
| | Q147R Q169Y | | | |
| | F183V | | | |
| 66 | SEQ ID NO: 5 | SEQ ID NO: 173 + | SEQ ID NO: 34 | 1.8 |
| | having mutations | S34H + L37R | | |
| | A32S S56A | | | |
| | N134D A146D | | | |
| | Q147R Q169Y | | | |
| | F183V | | | |
| 67 | SEQ ID NO: 5 | SEQ ID NO: 173 + | SEQ ID NO: 35 | 2.1 |
| | having mutations | S34H + L37R | | |
| | A32S S56A | | | |
| | N134D A146D | | | |
| | Q147R Q169Y | | | |
| | F183V | | | |
| 68 | SEQ ID NO: 5 | SEQ ID NO: 173 + | SEQ ID NO: 36 | 1.9 |
| | having mutations | S34H + L37R | | |
| | A32S S56A | | | |
| | N134D A146D | | | |
| | Q147R Q169Y | | | |
| | F183V | | | |
| 69 | SEQ ID NO: 5 | SEQ ID NO: 173 + | SEQ ID NO: 38 | 2.0 |
| | having mutations | S34H + L37R | | |
| | A32S S56A | | | |
| | N134D A146D | | | |
| | Q147R Q169Y | | | |
| | F183V | | | |
| 70 | SEQ ID NO: 5 | SEQ ID NO: 173 + | SEQ ID NO: 39 | 2.6 |
| | having mutations | S34H + L37R | | |
| | A32S S56A | | | |
| | N134D A146D | | | |
| | Q147R Q169Y | | | |
| | F183V | | | |
| 71 | SEQ ID NO: 5 | SEQ ID NO: 173 + | SEQ ID NO: 40 | 2.9 |
| | having mutations | S34H + L37R | | |
| | A32S S56A | | | |
| | N134D A146D | | | |
| | Q147R Q169Y | | | |
| | F183V | | | |
| 72 | SEQ ID NO: 5 | SEQ ID NO: 173 + | SEQ ID NO: 43 | 1.6 |
| | having mutations | S34H + L37R | | |
| | A32S S56A | | | |
| | N134D A146D | | | |
| | Q147R Q169Y | | | |
| | F183V | | | |
| 73 | SEQ ID NO: 5 | SEQ ID NO: 173 + | SEQ ID NO: 46 | 2.1 |
| | having mutations | S34H + L37R | | |
| | A32S S56A | | | |
| | N134D A146D | | | |
| | Q147R Q169Y | | | |
| | F183V | | | |
| 8 | SEQ ID NO: 5 | SEQ ID NO: 173 + | SEQ ID NO: 30 | 3.1 |
| | having mutations | S34H + L37R | | |
| | A32S S56A | | | |
| | N134D A146D | | | |
| | Q147R Q169Y | | | |
| | F183V | | | |

Notation with respect to CM: SEQ ID NO followed by “+” indicates that the specified mutation has been introduced in the referenced sequence. E.g. 173+S34H, L37R indicates that S in position 34 of SEQ ID NO:173 has been substituted with H and L in position 37 of SEQ ID NO: 173 has been substituted by R.

The results demonstrate that improved stability can be obtained when the different proline rich linkers are combined with a variant of SEQ ID NO: 173.

### Example 8: Variants of CBM SEQ ID NO:173 with P7G Linker (SEQ ID NO:30)

Variants of OEM having SEQ ID NO:173 comprising the mutations S34H, L37R and at least one further mutation with P7G linker (SEQ ID NO:30) were tested as described in the example 2, Linker Stability Assay, with the following modifications: the detergent was Model Detergent A2, the protease SEQ ID NO. 10. The variants were incubated at 45° C. for 3 days in the detergent with the protease before being tested for residual activity and ability to bind to cellulose

| | | CBM | | Relative |
|---|---|---|---|---|
| Example | Catalytic domain | (SEQ ID NO | Linker | linker |
| ID | (SEQ ID NO) | and mutation) | (SEQ ID NO) | stability |
| 74 | SEQ ID NO: 5 | SEQ ID NO: 7 | SEQ ID NO: 30 | 1.0 |
| | having mutations A32S | | | |
| | S56A N134D | | | |
| | A146D | | | |
| | Q147R | | | |
| | Q169Y | | | |
| | F183V | | | |
| 75 | SEQ ID NO: 5 | SEQ ID NO: 173 + A22E, S34H, | SEQ ID NO: 30 | 2.7 |
| | having mutations A32S | L37R | | |
| | S56A N134D | | | |
| | A146D | | | |
| | Q147R | | | |
| | Q169Y | | | |
| | F183V | | | |
| 76 | SEQ ID NO: 5 | SEQ ID NO: 173 + A22N + S34H + | SEQ ID NO: 30 | 2.6 |
| | having mutations A32S | L37R | | |
| | S56A N134D | | | |
| | A146D | | | |
| | Q147R | | | |
| | Q169Y | | | |
| | F183V | | | |
| 77 | SEQ ID NO: 5 | SEQ ID NO: 173 + A22P + S34H + | SEQ ID NO: 30 | 2.9 |
| | having mutations A32S | L37R | | |
| | S56A N134D | | | |
| | A146D | | | |
| | Q147R | | | |
| | Q169Y | | | |
| | F183V | | | |
| 78 | SEQ ID NO: 5 | SEQ ID NO: 173 + Q28P + S34H + | SEQ ID NO: 30 | 2.4 |
| | having mutations A32S | L37R | | |
| | S56A N134D | | | |
| | A146D | | | |
| | Q147R | | | |
| | Q169Y | | | |
| | F183V | | | |
| 79 | SEQ ID NO: 5 | SEQ ID NO: 173 + T18R + S34H +, | SEQ ID NO: 30 | 2.8 |
| | having mutations A32S | L37R | | |
| | S56A N134D | | | |
| | A146D | | | |
| | Q147R | | | |
| | Q169Y | | | |
| | F183V | | | |
| 80 | SEQ ID NO: 5 | SEQ ID NO: 173 + T25E + S34H + | SEQ ID NO: 30 | 2.8 |
| | having mutations A32S | L37R | | |
| | S56A N134D | | | |
| | A146D | | | |
| | Q147R | | | |
| | Q169Y | | | |
| | F183V | | | |
| 81 | SEQ ID NO: 5 | SEQ ID NO: 173 + T27I + S34H + | SEQ ID NO: 30 | 2.0 |
| | having mutations A32S | L37R | | |
| | S56A N134D | | | |
| | A146D | | | |
| | Q147R | | | |
| | Q169Y | | | |
| | F183V | | | |
| 82 | SEQ ID NO: 5 | SEQ ID NO: 173 + V21E + S34H + | SEQ ID NO: 30 | 4.1 |
| | having mutations A32S | L37R | | |
| | S56A N134D | | | |
| | A146D | | | |
| | Q147R | | | |
| | Q169Y | | | |
| | F183V | | | |
| 83 | SEQ ID NO: 5 | SEQ ID NO: 173 + V21E + A22E + | SEQ ID NO: 30 | 3.5 |
| | having mutations A32S | S34H + L37R | | |
| | S56A N134D | | | |
| | A146D | | | |
| | Q147R | | | |
| | Q169Y | | | |
| | F183V | | | |
| 84 | SEQ ID NO: 5 | SEQ ID NO: 173 + T18K + S34H + | SEQ ID NO: 30 | 1.9 |
| | having mutations A32S | L37R | | |
| | S56A N134D | | | |
| | A146D | | | |
| | Q147R | | | |
| | Q169Y | | | |
| | F183V | | | |
| 85 | SEQ ID NO: 5 | SEQ ID NO: 173 + Y14W + T18K + | SEQ ID NO: 30 | 1.8 |
| | having mutations A32S | S34H + L37R | | |
| | S56A N134D | | | |
| | A146D | | | |
| | Q147R | | | |
| | Q169Y | | | |
| | F183V | | | |
| 86 | SEQ ID NO: 5 | SEQ ID NO: 173 + T18K + A22E + | SEQ ID NO: 30 | 2.3 |
| | having mutations A32S | S34H + L37R | | |
| | S56A N134D | | | |
| | A146D | | | |
| | Q147R | | | |
| | Q169Y | | | |
| | F183V | | | |
| 87 | SEQ ID NO: 5 | SEQ ID NO: 173 + T18K + A22N + | SEQ ID NO: 30 | 1.8 |
| | having mutations A32S | S34H + L37R | | |
| | S56A N134D | | | |
| | A146D | | | |
| | Q147R | | | |
| | Q169Y | | | |
| | F183V | | | |
| 88 | SEQ ID NO: 5 | SEQ ID NO: 173 + T18K + A22P + | SEQ ID NO: 30 | 2.5 |
| | having mutations A32S | S34H + L37R | | |
| | S56A N134D | | | |
| | A146D | | | |
| | Q147R | | | |
| | Q169Y | | | |
| | F183V | | | |
| 89 | SEQ ID NO: 5 | SEQ ID NO: 173 + T18K, V21E, | SEQ ID NO: 30 | 2.4 |
| | having mutations A32S | A22N, S34H, L37R | | |
| | S56A N134D | | | |
| | A146D | | | |
| | Q147R | | | |
| | Q169Y | | | |
| | F183V | | | |
| 90 | SEQ ID NO: 5 | SEQ ID NO: 173 + T18K+ Q28P + | SEQ ID NO: 30 | 2.2 |
| | having mutations A32S | S34H + L37R | | |
| | S56A N134D | | | |
| | A146D | | | |
| | Q147R | | | |
| | Q169Y | | | |
| | F183V | | | |
| 91 | SEQ ID NO: 5 | SEQ ID NO: 173 + T18K + T25E + | SEQ ID NO: 30 | 2.0 |
| | having mutations A32S | S34H + L37R | | |
| | S56A N134D | | | |
| | A146D | | | |
| | Q147R | | | |
| | Q169Y | | | |
| | F183V | | | |
| 92 | SEQ ID NO: 5 | SEQ ID NO: 173 + T18K + T27I + | SEQ ID NO: 30 | 2.3 |
| | having mutations A32S | S34H + L37R | | |
| | S56A N134D | | | |
| | A146D | | | |
| | Q147R | | | |
| | Q169Y | | | |
| | F183V | | | |
| 93 | SEQ ID NO: 5 | SEQ ID NO: 173 + T18K + V21E + | SEQ ID NO: 30 | 2.8 |
| | having mutations A32S | S34H + L37R | | |
| | S56A N134D | | | |
| | A146D | | | |
| | Q147R | | | |
| | Q169Y | | | |
| | F183V | | | |
| 94 | SEQ ID NO: 5 | SEQ ID NO: 173 + T18K + V21E + | SEQ ID NO: 30 | 2.5 |
| | having mutations A32S | A22E + S34H + L37R | | |
| | S56A N134D | | | |
| | A146D | | | |
| | Q147R | | | |
| | Q169Y | | | |
| | F183V | | | |
| 95 | SEQ ID NO: 5 | SEQ ID NO: 173 + T18K + S34Y + | SEQ ID NO: 30 | 3.8 |
| | having mutations A32S | L37R | | |
| | S56A N134D | | | |
| | A146D | | | |
| | Q147R | | | |
| | Q169Y | | | |
| | F183V | | | |
| 96 | SEQ ID NO: 5 | SEQ ID NO: 173 + Y14W + T18K + | SEQ ID NO: 30 | 2.1 |
| | having | A22N + S34H + L37R | | |
| | mutations | | | |
| | A32S S56A | | | |
| | N134D | | | |
| | A146D | | | |
| | Q147R | | | |
| | Q169Y | | | |
| | F183V | | | |
| 97 | SEQ ID NO: 5 | SEQ ID NO: 173 + Y14W + S34H + | SEQ ID NO: 30 | 1.4 |
| | having mutations A32S | L37R | | |
| | S56A N134D | | | |
| | A146D | | | |
| | Q147R | | | |
| | Q169Y | | | |
| | F183V | | | |

Notation with respect to CBM: SEQ ID NO followed by “+” indicates that the specified mutation has been introduced in the referenced sequence. E.g. 173+A22E, S34H, L37R indicates that A in position 22 of SEQ ID NO:173 has been substituted with E, S in position 34 of SEQ ID NO:173 has been substituted with H and L in position 37 of SEQ ID NO: 173 has been substituted by R.

The results demonstrate that improved stability relative to the CBM having SEQ ID NO:7 can be obtained even when introducing mutations in SEQ ID NO:173 at positions selected from 14, 18, 21, 22, 25, 27, 28, 34, and 37.

1. A variant of a carbohydrate binding module, comprising a substitution at one or more positions corresponding to positions 34, 37, 14, 18, 21, 22, 25, 27, 28, 29 of the polypeptide of SEQ ID NO: 173, wherein the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity, but less than 100% sequence identity, to the polypeptide of SEQ ID NO: 173; and wherein the variant has carbohydrate binding activity.

2. The variant of claim 1, which is a variant of a carbohydrate binding module Family 1 (CBM1).

3. The variant of claim 1, which comprises at least one substitution selected from the group consisting of:
a substitution of the amino acid residue at position 34 with H, Y, K, or R;
a substitution of the amino acid residue at position 37 with R;
a substitution of the amino acid residue at position 14 with W;
a substitution of the amino acid residue at position 18 with K, or R;
a substitution of the amino acid residue at position 21 with E;
a substitution of the amino acid residue at position 22 with E, N, or P;
a substitution of the amino acid residue at position 25 with E;
a substitution of the amino acid residue at position 27 with I;
a substitution of the amino acid residue at position 28 with P; and
a substitution of the amino acid residue at position 29 with P.

4. The variant of claim 3, which comprises at least one substitution selected from the group consisting of:
S34H; S34Y; S34K; S34R;
L37R;
Y14W;
T18K; T18R;
V21E;
A22E; A22P; A22N;
T25E;
T27I;
Q28P; and
L29P.

5. The variant of claim 1, which comprises at least two substitutions selected from the group consisting of:
a substitution of the amino acid residue at position 34 with H, Y, K, R, preferably H, Y;
a substitution of the amino acid residue at position 37 with R
a substitution of the amino acid residue at position 14 with W;
a substitution of the amino acid residue at position 18 with K, R;
a substitution of the amino acid residue at position 21 with E;
a substitution of the amino acid residue at position 22 with E, P;
a substitution of the amino acid residue at position 25 with E;
a substitution of the amino acid residue at position 27 with I;
a substitution of the amino acid residue at position 28 with P; and
a substitution of the amino acid residue at position 29 with P.

6. The variant of claim 5, which comprises at least two substitutions selected from the group consisting of:
S34H; S34Y; S34K; S34R;
L37R;
Y14W;
T18K; T18R;
V21E;
A22E; A22N; A22P;
T25E;
T27I;
Q28P; and
L29P.

7. The variant of claim 1, comprising a substitution selected from the group consisting of:
S34H
S34Y
S34K
S34R
Y14W
T18K
L37R
S34Y,L37R
T18K,S34H
S34H,L37R
T18K,S34Y,L37R
T18R
V21E
A22E
A22P
A22N
T25E
T27I
Q28P
L29P
S34H,L37R,Y14W
S34H,L37R,T18K
S34H,L37R,Y14W,T18K
S34H,L37R,T18R
S34H,L37R,V21E
S34H,L37R,A22E
S34H,L37R,A22P
S34H,L37R,A22N
S34H,L37R,Q28P
S34H,L37R,L29P
S34H,L37R,T25E
S34H,L37R,T27I
S34H,L37R,V21E,A22E
S34H,L37R,V21E,A22N
Y14W,T18R,S34H,L37R
Y14W,V21E,S34H,L37R
Y14W,A22E,S34H,L37R
Y14W,A22P,S34H,L37R
Y14W,A22N,S34H,L37R
Y14W,Q18P,S34H,L37R
Y14W,L29P,S34H,L37R
Y14W,T25E,S34H,L37R
Y14W,T27I,S34H,L37R
Y14W,V21E,A22E,S34H,L37R
Y14W,V21E,A22N,S34H,L37R
T18K,V21E,S34H,L37R
T18K,A22E,S34H,L37R
T18K,A22P,S34H,L37R
T18K,A22N,S34H,L37R
T18K,Q28P,S34H,L37R
T18K,L29P,S34H,L37R
T18K,T25E,S34H,L37R
T18K,T27I,S34H,L37R
T18K,V21E,A22E,S34H,L37R
T18K,V21E,A22N,S34H,L37R
Y14W,T18K,V21E,S34H,L37R
Y14W,T18K,A22E,S34H,L37R
Y14W,T18K,A22P,S34H,L37R
Y14W,T18K,A22N,S34H,L37R
Y14W,T18K,Q28P,S34H,L37R
Y14W,T18K,L29P,S34H,L37R
Y14W,T18K,T25E,S34H,L37R
Y14W,T18K,T27I,S34H,L37R
Y14W,T18K,V21E,A22E,S34H,L37R
Y14W,T18K,A21E,A22N,S34H,L37R

8. The variant of claim 1, further comprising one or more catalytic domains from a polypeptide having glycoside hydrolase activity and one or more linkers.

9. A cellulase variant comprising the CBM variant of claim 1.

10. A variant having glycoside hydrolase activity, comprising (a) one or more catalytic domains from a polypeptide having glycoside hydrolase activity; (b) one or more linkers; and (c) one or more carbohydrate binding module variant of claim 1.

11. The glycoside hydrolase variant of claim 10, wherein the linker is a proline-rich linker.

12. The glycoside hydrolase variant of claim 11, wherein the proline-rich linker is selected from the group consisting of
(SEQ ID NO: 12)
TTPPTPTPTPTPG
(SEQ ID NO: 13)
TTPTPPTPTPTPTPG
(SEQ ID NO: 14)
TTPTPTPPTPTPTPTPG
(SEQ ID NO: 15)
TTPTPTPTPPTPTPTPTPG
(SEQ ID NO: 16)
TPPTPPTPPTPPTPPTPPTPPTPPTPPTPPTPPG
(SEQ ID NO: 17)
TPTTPTTPTTPTG
(SEQ ID NO: 18)
TPTTPTTPTTPTTPTTPTG
(SEQ ID NO: 19)
SPSSPSSPSSPSG
(SEQ ID NO: 20)
SPSSPSSPSSPSSPSG
(SEQ ID NO: 21)
SPPSPPSPPSPPSPPG
(SEQ ID NO: 22)
SPPSPPSPPSPPSPPSPPSPPSPPSPPSPPG
(SEQ ID NO: 23)
PPSSPSSPSSPSSPSSPSSPSG
(SEQ ID NO: 24)
SPSPG
(SEQ ID NO: 25)
SPSPSPSPSPG
(SEQ ID NO: 26)
TPTPTPTPTPG
(SEQ ID NO: 27)
PPPP
(SEQ ID NO: 28)
PPPPP
(SEQ ID NO: 29)
PPPPPP
(SEQ ID NO: 30)
PPPPPPPG
(SEQ ID NO: 31)
PPPPPPP
(SEQ ID NO: 32)
PPPPPPPPG
(SEQ ID NO: 33)
PPPPPPPPPG
(SEQ ID NO: 34)
PPPPPPPPPPG
(SEQ ID NO: 35)
PPPPPPPPPPPG
(SEQ ID NO: 36)
PPPPPPPPPPPPPG
(SEQ ID NO: 37)
PEPTPEPTG
(SEQ ID NO: 38)
PEPTPEPTPEPTG
(SEQ ID NO: 39)
PEPTPEPTPEPTPEPTG
(SEQ ID NO: 40)
PEPTPEPTPEPTPEPTPEPTG
(SEQ ID NO: 41)
PSPTPSPTPSPTPSPTG
(SEQ ID NO: 42)
PSPTPSPTPSPTPSPTPSPTG
(SEQ ID NO: 43)
PQPTPQPTG
(SEQ ID NO: 44)
PDPTPDPTG
(SEQ ID NO: 45)
PRPTPEPTG
(SEQ ID NO: 46)
PQPTPEPTG
(SEQ ID NO: 47)
PSPNSPNSPNG
(SEQ ID NO: 48)
PEPTPRPTG
(SEQ ID NO: 49)
PQPTPEPTPQPTPEPTPQPTPEPTPQPTG
(SEQ ID NO: 50)
PDPTPDPTPDPTG
(SEQ ID NO: 51)
PQPTPQPTPQPTPQPTG
(SEQ ID NO: 52)
PQPTPEPTPQPTPEPTG
(SEQ ID NO: 53)
SPSPSPSPPPG
(SEQ ID NO: 54)
SPSPSPSPDPG
(SEQ ID NO: 55)
SPSPSPSPKPG
(SEQ ID NO: 56)
SPSPSPSPAPG
(SEQ ID NO: 57)
SPSPSPSPSPSG
(SEQ ID NO: 58)
SPSPSPSPSP
(SEQ ID NO: 59)
SPSPSPSPSPS
(SEQ ID NO: 60)
SPSPSPSPSPP
(SEQ ID NO: 61)
SPSPSPSPSPE
(SEQ ID NO: 62)
SPSPSPSPSPN
(SEQ ID NO: 63)
SPSPSPSPSPGG
(SEQ ID NO: 64)
SPSPSPSPSPK
(SEQ ID NO: 65)
PEPTPEPTP
(SEQ ID NO: 66)
PEPTPEPTR
(SEQ ID NO: 67)
PEPTPEPTPEPTP
(SEQ ID NO: 68)
PEPTPEPTPEPTPEPTPSPTG
(SEQ ID NO: 69)
PEPTPEPTPEPTPEPTPTPTG
(SEQ ID NO: 70)
PEPTPEPTPEPTPEPTPGPTG
(SEQ ID NO: 71)
PEPTPEPTPEPTPEPTPDPTG
(SEQ ID NO: 72)
PEPTPEPTPEPTPEPTPETG
(SEQ ID NO: 73)
PEPTPEPTPEPTPEPTPEPTD
(SEQ ID NO: 74)
PEPTPEPTE
(SEQ ID NO: 75)
PEPTPEPTPEPTPEPTPEP
(SEQ ID NO: 76)
PEPTPEPTPEPTPEPTPSPT
(SEQ ID NO: 77)
PEPTPEPTPEPTPEPTPRPTT
(SEQ ID NO: 78)
PEPTPEPTPEPTPEPTPEPTT
(SEQ ID NO: 79)
PEPTPEPTPEPTPEPTPEPT
(SEQ ID NO: 80)
PEPTPEPTPEPTPEPTPEPTS
(SEQ ID NO: 81)
PEPTPEPTPEPTPEPTPEPTR
(SEQ ID NO: 82)
PPPGGPGGPGTPTSTAPGSGPTSPGGGSG
(SEQ ID NO: 83)
PPPGGPGGTGTPTSTAPGSGPTSPGGGSG
(SEQ ID NO: 84)
PPSGGPGGPGTPTSTAPGSGPTSPGGGSG
(SEQ ID NO: 85)
PEPTPRPTPEPTPRPTG
(SEQ ID NO: 86)
PKPTPEPTPKPTPEPTG
(SEQ ID NO: 87)
PEPTPKPTPEPTPKPTG
(SEQ ID NO: 88)
PEPTPQPTPEPTPQPTG
(SEQ ID NO: 89)
PRPTPEPTPRPTG
(SEQ ID NO: 90)
PKPTPEPTPKPTG
(SEQ ID NO: 91)
PEPTPQPTG
(SEQ ID NO: 92)
PEPTPQPTPEPTG
(SEQ ID NO: 93)
TPPTPPG
(SEQ ID NO: 94)
SPSSPSG
(SEQ ID NO: 95)
SPSSPSSPSG
(SEQ ID NO: 96)
TPTTPTG
(SEQ ID NO: 97)
TPTTPTTPTG

13. The glycoside hydrolase variant of claim 10, wherein the linker comprises PPPPPPP (SEQ ID NO: 31), PPPPPPPG (SEQ ID NO: 30), SPSPSPSPSP (SEQ ID NO: 58) or SPSPSPSPSPG (SEQ ID NO: 25).

14. The glycoside hydrolase variant of claim 10, which is selected from the group consisting of:

| | Variant Concept | | |
|---|---|---|---|
| | Catalytic domain (N-terminal) | Linker | CBM (C-terminal) |
| Variant 1 | SEQ ID NO: 5 having mutations | PPPPPPPG | SEQ ID NO: 173 having mutation |
| | A32S S56A N134D A146D | (SEQ ID NO: 30) | Y14W |
| | Q147R Q169Y F183V | | |
| Variant 2 | SEQ ID NO: 5 having mutations | PPPPPPPG | SEQ ID NO: 173 having mutation |
| | A32S S56A N134D A146D | (SEQ ID NO: 30) | S34H |
| | Q147R Q169Y F183V | | |
| Variant 3 | SEQ ID NO: 5 having mutations | PPPPPPPG | SEQ ID NO: 173 having mutation |
| | A32S S56A N134D A146D | (SEQ ID NO: 30) | S34Y |
| | Q147R Q169Y F183V | | |
| Variant 4 | SEQ ID NO: 5 having mutations | PPPPPPPG | SEQ ID NO: 173 having mutation |
| | A32S S56A N134D A146D | (SEQ ID NO: 30) | S34K |
| | Q147R Q169Y F183V | | |
| Variant 5 | SEQ ID NO: 5 having mutations | PPPPPPPG | SEQ ID NO: 173 having mutation |
| | A32S S56A N134D A146D | (SEQ ID NO: 30) | S34R |
| | Q147R Q169Y F183V | | |
| Variant 6 | SEQ ID NO: 5 having mutations | PPPPPPPG | SEQ ID NO: 173 having mutation |
| | A32S S56A N134D A146D | (SEQ ID NO: 30) | T18K |
| | Q147R Q169Y F183V | | |
| Variant 7 | SEQ ID NO: 5 having mutations | PPPPPPPG | SEQ ID NO: 173 having mutation |
| | A32S S56A N134D A146D | (SEQ ID NO: 30) | L37R |
| | Q147R Q169Y F183V | | |
| Variant 8 | SEQ ID NO: 5 having mutations | PPPPPPPG | SEQ ID NO: 173 having mutations |
| | A32S S56A N134D A146D | (SEQ ID NO: 30) | S34Y, L37R |
| | Q147R Q169Y F183V | | |
| Variant 9 | SEQ ID NO: 5 having mutations | PPPPPPPG | SEQ ID NO: 173 having mutations |
| | A32S S56A N134D A146D | (SEQ ID NO: 30) | T18K, S34H |
| | Q147R Q169Y F183V | | |
| Variant 10 | SEQ ID NO: 5 having mutations | PPPPPPPG | SEQ ID NO: 173 having mutations |
| | A32S S56A N134D A146D | (SEQ ID NO: 30) | S34H, L37R |
| | Q147R Q169Y F183V | | |
| Variant 11 | SEQ ID NO: 5 having mutations | PPPPPPPG | SEQ ID NO: 173 having mutations |
| | A32S S56A N134D A146D | (SEQ ID NO: 30) | T18K, S34Y, L37R |
| | Q147R Q169Y F183V | | |
| Variant 12 | SEQ ID NO: 5 having mutations | PPPPPPPG | SEQ ID NO: 173 having mutation |
| | A32S S56A N134D A146D | (SEQ ID NO: 30) | T18R |
| | Q147R Q169Y F183V | | |
| Variant 13 | SEQ ID NO: 5 having mutations | PPPPPPPG | SEQ ID NO: 173 having mutation |
| | A32S S56A N134D A146D | (SEQ ID NO: 30) | V21E |
| | Q147R Q169Y F183V | | |
| Variant 14 | SEQ ID NO: 5 having mutations | PPPPPPPG | SEQ ID NO: 173 having mutation |
| | A32S S56A N134D A146D | (SEQ ID NO: 30) | A22E |
| | Q147R Q169Y F183V | | |
| Variant 15 | SEQ ID NO: 5 having mutations | PPPPPPPG | SEQ ID NO: 173 having mutation |
| | A32S S56A N134D A146D | (SEQ ID NO: 30) | A22P |
| | Q147R Q169Y F183V | | |
| Variant 16 | SEQ ID NO: 5 having mutations | PPPPPPPG | SEQ ID NO: 173 having mutation |
| | A32S S56A N134D A146D | (SEQ ID NO: 30) | A22N |
| | Q147R Q169Y F183V | | |
| Variant 17 | SEQ ID NO: 5 having mutations | PPPPPPPG | SEQ ID NO: 173 having mutation |
| | A32S S56A N134D A146D | (SEQ ID NO: 30) | T25E |
| | Q147R Q169Y F183V | | |
| Variant 18 | SEQ ID NO: 5 having mutations | PPPPPPPG | SEQ ID NO: 173 having mutation |
| | A32S S56A N134D A146D | (SEQ ID NO: 30) | T27I |
| | Q147R Q169Y F183V | | |
| Variant 19 | SEQ ID NO: 5 having mutations | PPPPPPPG | SEQ ID NO: 173 having mutation |
| | A32S S56A N134D A146D | (SEQ ID NO: 30) | Q28P |
| | Q147R Q169Y F183V | | |
| Variant 20 | SEQ ID NO: 5 having mutations | PPPPPPPG | SEQ ID NO: 173 having mutation |
| | A32S S56A N134D A146D | (SEQ ID NO: 30) | L29P |
| | Q147R Q169Y F183V | | |
| Variant 21 | SEQ ID NO: 5 having mutations | PPPPPPPG | SEQ ID NO: 173 having mutations |
| | A32S S56A N134D A146D | (SEQ ID NO: 30) | S34H, L37R, Y14W |
| | Q147R Q169Y F183V | | |
| Variant 22 | SEQ ID NO: 5 having mutations | PPPPPPPG | SEQ ID NO: 173 having mutations |
| | A32S S56A N134D A146D | (SEQ ID NO: 30) | S34H, L37R, T18K |
| | Q147R Q169Y F183V | | |
| Variant 23 | SEQ ID NO: 5 having mutations | PPPPPPPG | SEQ ID NO: 173 having mutations |
| | A32S S56A N134D A146D | (SEQ ID NO: 30) | S34H, L37R, Y14W, T18K |
| | Q147R Q169Y F183V | | |
| Variant 24 | SEQ ID NO: 5 having mutations | PPPPPPPG | SEQ ID NO: 173 having mutations |
| | A32S S56A N134D A146D | (SEQ ID NO: 30) | S34H, L37R, T18R |
| | Q147R Q169Y F183V | | |
| Variant 25 | SEQ ID NO: 5 having mutations | PPPPPPPG | SEQ ID NO: 173 having mutations |
| | A32S S56A N134D A146D | (SEQ ID NO: 30) | S34H, L37R, V21E |
| | Q147R Q169Y F183V | | |
| Variant 26 | SEQ ID NO: 5 having mutations | PPPPPPPG | SEQ ID NO: 173 having mutations |
| | A32S S56A N134D A146D | (SEQ ID NO: 30) | S34H, L37R, A22E |
| | Q147R Q169Y F183V | | |
| Variant 27 | SEQ ID NO: 5 having mutations | PPPPPPPG | SEQ ID NO: 173 having mutations |
| | A32S S56A N134D A146D | (SEQ ID NO: 30) | S34H, L37R, A22P |
| | Q147R Q169Y F183V | | |
| Variant 28 | SEQ ID NO: 5 having mutations | PPPPPPPG | SEQ ID NO: 173 having mutations |
| | A32S S56A N134D A146D | (SEQ ID NO: 30) | S34H, L37R, A22N |
| | Q147R Q169Y F183V | | |
| Variant 29 | SEQ ID NO: 5 having mutations | PPPPPPPG | SEQ ID NO: 173 having mutations |
| | A32S S56A N134D A146D | (SEQ ID NO: 30) | S34H, L37R, Q28P |
| | Q147R Q169Y F183V | | |
| Variant 30 | SEQ ID NO: 5 having mutations | PPPPPPPG | SEQ ID NO: 173 having mutations |
| | A32S S56A N134D A146D | (SEQ ID NO: 30) | S34H, L37R, L29P |
| | Q147R Q169Y F183V | | |
| Variant 31 | SEQ ID NO: 5 having mutations | PPPPPPPG | SEQ ID NO: 173 having mutations |
| | A32S S56A N134D A146D | (SEQ ID NO: 30) | S34H, L37R, T25E |
| | Q147R Q169Y F183V | | |
| Variant 32 | SEQ ID NO: 5 having mutations | PPPPPPPG | SEQ ID NO: 173 having mutations |
| | A32S S56A N134D A146D | (SEQ ID NO: 30) | S34H, L37R, T27I |
| | Q147R Q169Y F183V | | |
| Variant 33 | SEQ ID NO: 5 having mutations | PPPPPPPG | SEQ ID NO: 173 having mutations |
| | A32S S56A N134D A146D | (SEQ ID NO: 30) | S34H, L37R, V21E, A22E |
| | Q147R Q169Y F183V | | |
| Variant 34 | SEQ ID NO: 5 having mutations | PPPPPPPG | SEQ ID NO: 173 having mutations |
| | A32S S56A N134D A146D | (SEQ ID NO: 30) | S34H, L37R, V21E, A22N |
| | Q147R Q169Y F183V | | |
| Variant 35 | SEQ ID NO: 5 having mutations | PPPPPPPG | SEQ ID NO: 173 having mutations |
| | A32S S56A N134D A146D | (SEQ ID NO: 30) | Y14W, T18R, S34H, L37R |
| | Q147R Q169Y F183V | | |
| Variant 36 | SEQ ID NO: 5 having mutations | PPPPPPPG | SEQ ID NO: 173 having mutations |
| | A32S S56A N134D A146D | (SEQ ID NO: 30) | Y14W, V21E, S34H, L37R |
| | Q147R Q169Y F183V | | |
| Variant 37 | SEQ ID NO: 5 having mutations | PPPPPPPG | SEQ ID NO: 173 having mutations |
| | A32S S56A N134D A146D | (SEQ ID NO: 30) | Y14W, A22E, S34H, L37R |
| | Q147R Q169Y F183V | | |
| Variant 38 | SEQ ID NO: 5 having mutations | PPPPPPPG | SEQ ID NO: 173 having mutations |
| | A32S S56A N134D A146D | (SEQ ID NO: 30) | Y14W, A22P, S34H, L37R |
| | Q147R Q169Y F183V | | |
| Variant 39 | SEQ ID NO: 5 having mutations | PPPPPPPG | SEQ ID NO: 173 having mutations |
| | A32S S56A N134D A146D | (SEQ ID NO: 30) | Y14W, A22N, S34H, L37R |
| | Q147R Q169Y F183V | | |
| Variant 40 | SEQ ID NO: 5 having mutations | PPPPPPPG | SEQ ID NO: 173 having mutations |
| | A32S S56A N134D A146D | (SEQ ID NO: 30) | Y14W, Q18P, S34H, L37R |
| | Q147R Q169Y F183V | | |
| Variant 41 | SEQ ID NO: 5 having mutations | PPPPPPPG | SEQ ID NO: 173 having mutations |
| | A32S S56A N134D A146D | (SEQ ID NO: 30) | Y14W, L29P, S34H, L37R |
| | Q147R Q169Y F183V | | |
| Variant 42 | SEQ ID NO: 5 having mutations | PPPPPPPG | SEQ ID NO: 173 having mutations |
| | A32S S56A N134D A146D | (SEQ ID NO: 30) | Y14W, T25E, S34H, L37R |
| | Q147R Q169Y F183V | | |
| Variant 43 | SEQ ID NO: 5 having mutations | PPPPPPPG | SEQ ID NO: 173 having mutations |
| | A32S S56A N134D A146D | (SEQ ID NO: 30) | Y14W, T27I, S34H, L37R |
| | Q147R Q169Y F183V | | |
| Variant 44 | SEQ ID NO: 5 having mutations | PPPPPPPG | SEQ ID NO: 173 having mutations |
| | A32S S56A N134D A146D | (SEQ ID NO: 30) | Y14W, V21E, A22E, S34H, L37R |
| | Q147R Q169Y F183V | | |
| Variant 45 | SEQ ID NO: 5 having mutations | PPPPPPPG | SEQ ID NO: 173 having mutations |
| | A32S S56A N134D A146D | (SEQ ID NO: 30) | Y14W, V21E, A22N, S34H, L37R |
| | Q147R Q169Y F183V | | |
| Variant 46 | SEQ ID NO: 5 having mutations | PPPPPPPG | SEQ ID NO: 173 having mutations |
| | A32S S56A N134D A146D | (SEQ ID NO: 30) | T18K, V21E, S34H, L37R |
| | Q147R Q169Y F183V | | |
| Variant 47 | SEQ ID NO: 5 having mutations | PPPPPPPG | SEQ ID NO: 173 having mutations |
| | A32S S56A N134D A146D | (SEQ ID NO: 30) | T18K, A22E, S34H, L37R |
| | Q147R Q169Y F183V | | |
| Variant 48 | SEQ ID NO: 5 having mutations | PPPPPPPG | SEQ ID NO: 173 having mutations |
| | A32S S56A N134D A146D | (SEQ ID NO: 30) | T18K, A22P, S34H, L37R |
| | Q147R Q169Y F183V | | |
| Variant 49 | SEQ ID NO: 5 having mutations | PPPPPPPG | SEQ ID NO: 173 having mutations |
| | A32S S56A N134D A146D | (SEQ ID NO: 30) | T18K, A22N, S34H, L37R |
| | Q147R Q169Y F183V | | |
| Variant 50 | SEQ ID NO: 5 having mutations | PPPPPPPG | SEQ ID NO: 173 having mutations |
| | A32S S56A N134D A146D | (SEQ ID NO: 30) | T18K, Q28P, S34H, L37R |
| | Q147R Q169Y F183V | | |
| Variant 51 | SEQ ID NO: 5 having mutations | PPPPPPPG | SEQ ID NO: 173 having mutations |
| | A32S S56A N134D A146D | (SEQ ID NO: 30) | T18K, L29P, S34H, L37R |
| | Q147R Q169Y F183V | | |
| Variant 52 | SEQ ID NO: 5 having mutations | PPPPPPPG | SEQ ID NO: 173 having mutations |
| | A32S S56A N134D A146D | (SEQ ID NO: 30) | T18K, T25E, S34H, L37R |
| | Q147R Q169Y F183V | | |
| Variant 53 | SEQ ID NO: 5 having mutations | PPPPPPPG | SEQ ID NO: 173 having mutations |
| | A32S S56A N134D A146D | (SEQ ID NO: 30) | T18K, T27I, S34H, L37R |
| | Q147R Q169Y F183V | | |
| Variant 54 | SEQ ID NO: 5 having mutations | PPPPPPPG | SEQ ID NO: 173 having mutations |
| | A32S S56A N134D A146D | (SEQ ID NO: 30) | T18K, V21E, A22E, S34H, L37R |
| | Q147R Q169Y F183V | | |
| Variant 55 | SEQ ID NO: 5 having mutations | PPPPPPPG | SEQ ID NO: 173 having mutations |
| | A32S S56A N134D A146D | (SEQ ID NO: 30) | T18K, V21E, A22N, S34H, L37R |
| | Q147R Q169Y F183V | | |
| Variant 56 | SEQ ID NO: 5 having mutations | PPPPPPPG | SEQ ID NO: 173 having mutations |
| | A32S S56A N134D A146D | (SEQ ID NO: 30) | Y14W, T18K, V21E, S34H, L37R |
| | Q147R Q169Y F183V | | |
| Variant 57 | SEQ ID NO: 5 having mutations | PPPPPPPG | SEQ ID NO: 173 having mutations |
| | A32S S56A N134D A146D | (SEQ ID NO: 30) | Y14W, T18K, A22E, S34H, L37R |
| | Q147R Q169Y F183V | | |
| Variant 58 | SEQ ID NO: 5 having mutations | PPPPPPPG | SEQ ID NO: 173 having mutations |
| | A32S S56A N134D A146D | (SEQ ID NO: 30) | Y14W, T18K, A22P, S34H, L37R |
| | Q147R Q169Y F183V | | |
| Variant 59 | SEQ ID NO: 5 having mutations | PPPPPPPG | SEQ ID NO: 173 having mutations |
| | A32S S56A N134D A146D | (SEQ ID NO: 30) | Y14W, T18K, A22N, S34H, L37R |
| | Q147R Q169Y F183V | | |
| Variant 60 | SEQ ID NO: 5 having mutations | PPPPPPPG | SEQ ID NO: 173 having mutations |
| | A32S S56A N134D A146D | (SEQ ID NO: 30) | Y14W, T18K, Q28P, S34H, L37R |
| | Q147R Q169Y F183V | | |
| Variant 61 | SEQ ID NO: 5 having mutations | PPPPPPPG | SEQ ID NO: 173 having mutations |
| | A32S S56A N134D A146D | (SEQ ID NO: 30) | Y14W, T18K, L29P, S34H, L37R |
| | Q147R Q169Y F183V | | |
| Variant 62 | SEQ ID NO: 5 having mutations | PPPPPPPG | SEQ ID NO: 173 having mutations |
| | A32S S56A N134D A146D | (SEQ ID NO: 30) | Y14W, T18K, T25E, S34H, L37R |
| | Q147R Q169Y F183V | | |
| Variant 63 | SEQ ID NO: 5 having mutations | PPPPPPPG | SEQ ID NO: 173 having mutations |
| | A32S S56A N134D A146D | (SEQ ID NO: 30) | Y14W, T18K, T27I, S34H, L37R |
| | Q147R Q169Y F183V | | |
| Variant 64 | SEQ ID NO: 5 having mutations | PPPPPPPG | SEQ ID NO: 173 having mutations |
| | A32S S56A N134D A146D | (SEQ ID NO: 30) | Y14W, T18K, V21E, A22E, S34H, L37R |
| | Q147R Q169Y F183V | | |
| Variant 65 | SEQ ID NO: 5 having mutations | PPPPPPPG | SEQ ID NO: 173 having mutations |
| | A32S S56A N134D A146D | (SEQ ID NO: 30) | Y14W, T18K, A21E, A22N, S34H, L37R |
| | Q147R Q169Y F183V | | |

15. The glycoside hydrolase variant of claim 10, having improved stability relative to the parent, such as in the presence of protease.

16. The glycoside hydrolase variant of claim 10, which has an improved property relative to the parent, preferably wherein the improved property is improved thermostability in the presence of protease.

17. A detergent composition comprising the glycoside hydrolase variant of claim 10.

18. An isolated polynucleotide encoding the glycoside hydrolase variant of claim 10.

19. A nucleic acid construct or expression vector comprising the polynucleotide of claim 18.

20. A recombinant host cell transformed with the polynucleotide of claim 18.

21. A method of producing a variant of a glycoside hydrolase variant, comprising:
a. cultivating the recombinant host cell of claim 20 under conditions suitable for expression of the variant; and
b. recovering the variant.

22. (canceled)

23. A method of pretreating a stained fabric or rejuvenating textile, the method comprising contacting the fabric or textile with a glycoside hydrolase variant of claim 10, wherein the pretreatment is effective to restore the visual and feel properties of the fabric or textile to match that of a new fabric or textile.


SEQUENCE LISTING (202 sequences)

SEQ ID NO: 1 - protein, 278 aa, Thielavia terrestris [sequence omitted]

SEQ ID NO: 2 - protein, 283 aa, Humicola insolens
ADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQISSSTSSPVNQPTSTSTTSTSTTSSPPVQPTTPSGCTAERWAQCGGNGWSGCTTCVAGSTCTKINDWYHQCL

SEQ ID NO: 3 - protein, 295 aa, Staphylotrichum coccosporum
ADGKSTRYWDCCKPSCSWPGKASVNQPVFACSANFQRISDPNVKSGCDGGSAYACADQTPWAVNDNFSYGFAATSISGGNEASWCCGCYELTFTSGPVAGKTMVVQSTSTGGDLGTNHFDLAMPGGGVGIFDGCSPQFGGLAGDRYGGVSSRSQCDSFPAALKPGCYWRFDWFKNADNPTFTFRQVQCPSELVARTGCRRNDDGNFPVFTPPSGGQSSSSSSSSSAKPTSTSTSTTSTKATSTTSTASSQTSSSTGGGCAAQRWAQCGGIGFSGCTTCVSGTTCNKQNDWYSQCL

SEQ ID NO: 4 - protein, 277 aa, Acremonium thermophilum
ALDGKSTRYWDCCKPSCGWPGKASVNQPVFSCSADWQRISDFNAKSGCDGGSAYSCADQTPWAVNDNFSYGFAATAIAGGSESSWCCACYALTFNSGPVAGKTMVVQSTSTGGDLGSNQFDLAIPGGGVGIFNGCASQFGGLPGAQYGGISDRSQCSSFPAPLQPGCQWRFDWFQNADNPTFTFQRVQCPSELTSRTGCKRDDDASYPVFNPPSGGSPSTTSTTTSSPSGPTGNPPGGGGCTAQKWAQCGGTGFTGCTTCVSGTTCQVQNQWYSQCL

SEQ ID NO: 5 - protein, 212 aa, Artificial Sequence
  Catalytic domain of SEQ ID NO 1
ASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDSSFPVFT

SEQ ID NO: 6 - protein, 37 aa, Artificial Sequence
  carbohydrate binding module of SEQ ID NO 1
CTSQKWAQCGGIGFSGCTTCVSGTTCQKLNDYYSQCL

SEQ ID NO: 7 - protein, 37 aa, Artificial Sequence
  carbohydrate binding module of SEQ ID NO 2
CTAERWAQCGGNGWSGCTTCVAGSTCTKINDWYHQCL

SEQ ID NO: 8 - protein, 37 aa, Artificial sequence
  Carbohydrate binding module of SEQ ID NO 3
CAAQRWAQCGGIGFSGCTTCVSGTTCNKQNDWYSQCL

SEQ ID NO: 9 - protein, 37 aa, Artificial Sequence
  Carbohydrate binding module of SEQ ID NO 4
CTAQKWAQCGGTGFTGCTTCVSGTTCQVQNQWYSQCL

SEQ ID NO: 10 - protein, 311 aa, Artificial Sequence
  Synthetic Construct
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK

SEQ ID NO: 11 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 12 - protein, 13 aa, Artificial Sequence
  Linker
TTPPTPTPTPTPG

SEQ ID NO: 13 - protein, 15 aa, Artificial Sequence
  Linker
TTPTPPTPTPTPTPG

SEQ ID NO: 14 - protein, 17 aa, Artificial Sequence
  Linker
TTPTPTPPTPTPTPTPG

SEQ ID NO: 15 - protein, 19 aa, Artificial Sequence
  Linker
TTPTPTPTPPTPTPTPTPG

SEQ ID NO: 16 - protein, 34 aa, Artificial Sequence
  Synthetic Construct
TPPTPPTPPTPPTPPTPPTPPTPPTPPTPPTPPG

SEQ ID NO: 17 - protein, 13 aa, Artificial Sequence
  Linker
TPTTPTTPTTPTG

SEQ ID NO: 18 - protein, 19 aa, Artificial Sequence
  Linker
TPTTPTTPTTPTTPTTPTG

SEQ ID NO: 19 - protein, 13 aa, Artificial Sequence
  Linker
SPSSPSSPSSPSG

SEQ ID NO: 20 - protein, 16 aa, Artificial Sequence
  Linker
SPSSPSSPSSPSSPSG

SEQ ID NO: 21 - protein, 16 aa, Artificial Sequence
  Linker
SPPSPPSPPSPPSPPG

SEQ ID NO: 22 - protein, 31 aa, Artificial Sequence
  Linker
SPPSPPSPPSPPSPPSPPSPPSPPSPPSPPG

SEQ ID NO: 23 - protein, 22 aa, Artificial Sequence
  Linker
PPSSPSSPSSPSSPSSPSSPSG

SEQ ID NO: 24 - protein, 5 aa, Artificial Sequence
  Linker
SPSPG

SEQ ID NO: 25 - protein, 11 aa, Artificial Sequence
  Linker
SPSPSPSPSPG

SEQ ID NO: 26 - protein, 11 aa, Artificial Sequence
  Linker
TPTPTPTPTPG

SEQ ID NO: 27 - protein, 4 aa, Artificial Sequence
  Linker
PPPP

SEQ ID NO: 28 - protein, 5 aa, Artificial Sequence
  Linker
PPPPP

SEQ ID NO: 29 - protein, 6 aa, Artificial Sequence
  Linker
PPPPPP

SEQ ID NO: 30 - protein, 8 aa, Artificial Sequence
  Linker
PPPPPPPG

SEQ ID NO: 31 - protein, 7 aa, Artificial Sequence
  Linker
PPPPPPP

SEQ ID NO: 32 - protein, 9 aa, Artificial Sequence
  Linker
PPPPPPPPG

SEQ ID NO: 33 - protein, 10 aa, Artificial Sequence
  Linker
PPPPPPPPPG

SEQ ID NO: 34 - protein, 11 aa, Artificial Sequence
  Linker
PPPPPPPPPPG

SEQ ID NO: 35 - protein, 12 aa, Artificial Sequence
  Linker
PPPPPPPPPPPG

SEQ ID NO: 36 - protein, 14 aa, Artificial Sequence
  Linker
PPPPPPPPPPPPPG

SEQ ID NO: 37 - protein, 9 aa, Artificial Sequence
  Linker
PEPTPEPTG

SEQ ID NO: 38 - protein, 13 aa, Artificial Sequence
  Linker
PEPTPEPTPEPTG

SEQ ID NO: 39 - protein, 17 aa, Artificial Sequence
  Linker
PEPTPEPTPEPTPEPTG

SEQ ID NO: 40 - protein, 21 aa, Artificial Sequence
  Linker
PEPTPEPTPEPTPEPTPEPTG

SEQ ID NO: 41 - protein, 17 aa, Artificial Sequence
  Linker
PSPTPSPTPSPTPSPTG

SEQ ID NO: 42 - protein, 21 aa, Artificial Sequence
  Linker
PSPTPSPTPSPTPSPTPSPTG

SEQ ID NO: 43 - protein, 9 aa, Artificial Sequence
  Linker
PQPTPQPTG

SEQ ID NO: 44 - protein, 9 aa, Artificial Sequence
  Linker
PDPTPDPTG

SEQ ID NO: 45 - protein, 9 aa, Artificial Sequence
  Linker
PRPTPEPTG

SEQ ID NO: 46 - protein, 9 aa, Artificial Sequence
  Linker
PQPTPEPTG

SEQ ID NO: 47 - protein, 11 aa, Artificial Sequence
  Linker
PSPNSPNSPNG

SEQ ID NO: 48 - protein, 9 aa, Artificial Sequence
  Linker
PEPTPRPTG

SEQ ID NO: 49 - protein, 29 aa, Artificial Sequence
  Linker
PQPTPEPTPQPTPEPTPQPTPEPTPQPTG

SEQ ID NO: 50 - protein, 13 aa, Artificial Sequence
  Linker
PDPTPDPTPDPTG

SEQ ID NO: 51 - protein, 17 aa, Artificial Sequence
  Linker
PQPTPQPTPQPTPQPTG

SEQ ID NO: 52 - protein, 17 aa, Artificial Sequence
  Linker
PQPTPEPTPQPTPEPTG

SEQ ID NO: 53 - protein, 11 aa, Artificial Sequence
  Linker
SPSPSPSPPPG

SEQ ID NO: 54 - protein, 11 aa, Artificial Sequence
  Linker
SPSPSPSPDPG

SEQ ID NO: 55 - protein, 11 aa, Artificial Sequence
  Linker
SPSPSPSPKPG

SEQ ID NO: 56 - protein, 11 aa, Artificial Sequence
  Linker
SPSPSPSPAPG

SEQ ID NO: 57 - protein, 12 aa, Artificial Sequence
  Linker
SPSPSPSPSPSG

SEQ ID NO: 58 - protein, 10 aa, Artificial Sequence
  Linker
SPSPSPSPSP

SEQ ID NO: 59 - protein, 11 aa, Artificial Sequence
  Linker
SPSPSPSPSPS

SEQ ID NO: 60 - protein, 11 aa, Artificial Sequence
  Linker
SPSPSPSPSPP

SEQ ID NO: 61 - protein, 11 aa, Artificial Sequence
  Linker
SPSPSPSPSPE

SEQ ID NO: 62 - protein, 11 aa, Artificial Sequence
  Linker
SPSPSPSPSPN

SEQ ID NO: 63 - protein, 12 aa, Artificial Sequence
  Linker
SPSPSPSPSPGG

SEQ ID NO: 64 - protein, 11 aa, Artificial Sequence
  Linker
SPSPSPSPSPK

SEQ ID NO: 65 - protein, 9 aa, Artificial Sequence
  Linker
PEPTPEPTP

SEQ ID NO: 66 - protein, 9 aa, Artificial Sequence
  Linker
PEPTPEPTR

SEQ ID NO: 67 - protein, 13 aa, Artificial Sequence
  Linker
PEPTPEPTPEPTP

SEQ ID NO: 68 - protein, 21 aa, Artificial Sequence
  Linker
PEPTPEPTPEPTPEPTPSPTG

SEQ ID NO: 69 - protein, 21 aa, Artificial Sequence
  Linker
PEPTPEPTPEPTPEPTPTPTG

SEQ ID NO: 70 - protein, 21 aa, Artificial Sequence
  Linker
PEPTPEPTPEPTPEPTPGPTG

SEQ ID NO: 71 - protein, 21 aa, Artificial Sequence
  Linker
PEPTPEPTPEPTPEPTPDPTG

SEQ ID NO: 72 - protein, 20 aa, Artificial Sequence
  Linker
PEPTPEPTPEPTPEPTPETG

SEQ ID NO: 73 - protein, 21 aa, Artificial Sequence
  Linker
PEPTPEPTPEPTPEPTPEPTD

SEQ ID NO: 74 - protein, 9 aa, Artificial Sequence
  Linker
PEPTPEPTE

SEQ ID NO: 75 - protein, 19 aa, Artificial Sequence
  Linker
PEPTPEPTPEPTPEPTPEP

SEQ ID NO: 76 - protein, 20 aa, Artificial Sequence
  Linker
PEPTPEPTPEPTPEPTPSPT

SEQ ID NO: 77 - protein, 21 aa, Artificial Sequence
  Linker
PEPTPEPTPEPTPEPTPRPTT

SEQ ID NO: 78 - protein, 21 aa, Artificial Sequence
  Linker
PEPTPEPTPEPTPEPTPEPTT

SEQ ID NO: 79 - protein, 20 aa, Artificial Sequence
  Linker
PEPTPEPTPEPTPEPTPEPT

SEQ ID NO: 80 - protein, 21 aa, Artificial Sequence
  Linker
PEPTPEPTPEPTPEPTPEPTS

SEQ ID NO: 81 - protein, 21 aa, Artificial Sequence
  Linker
PEPTPEPTPEPTPEPTPEPTR

SEQ ID NO: 82 - protein, 29 aa, Artificial Sequence
  Linker
PPPGGPGGPGTPTSTAPGSGPTSPGGGSG

SEQ ID NO: 83 - protein, 29 aa, Artificial Sequence
  Linker
PPPGGPGGTGTPTSTAPGSGPTSPGGGSG

SEQ ID NO: 84 - protein, 29 aa, Artificial Sequence
  Linker
PPSGGPGGPGTPTSTAPGSGPTSPGGGSG

SEQ ID NO: 85 - protein, 17 aa, Artificial Sequence
  Linker
PEPTPRPTPEPTPRPTG

SEQ ID NO: 86 - protein, 17 aa, Artificial Sequence
  Linker
PKPTPEPTPKPTPEPTG

SEQ ID NO: 87 - protein, 17 aa, Artificial Sequence
  Linker
PEPTPKPTPEPTPKPTG

SEQ ID NO: 88 - protein, 17 aa, Artificial Sequence
  Linker
PEPTPQPTPEPTPQPTG

SEQ ID NO: 89 - protein, 13 aa, Artificial Sequence
  Linker
PRPTPEPTPRPTG

SEQ ID NO: 90 - protein, 13 aa, Artificial Sequence
  Linker
PKPTPEPTPKPTG

SEQ ID NO: 91 - protein, 9 aa, Artificial Sequence
  Linker
PEPTPQPTG

SEQ ID NO: 92 - protein, 9 aa, Artificial Sequence
  Linker
PEPTPQPTG

SEQ ID NO: 93 - protein, 7 aa, Artificial Sequence
  Linker
TPPTPPG

SEQ ID NO: 94 - protein, 7 aa, Artificial Sequence
  Linker
SPSSPSG

SEQ ID NO: 95 - protein, 10 aa, Artificial Sequence
  Linker
SPSSPSSPSG

SEQ ID NO: 96 - protein, 7 aa, Artificial Sequence
  Linker
TPTTPTG

SEQ ID NO: 97 - protein, 10 aa, Artificial Sequence
  Linker
TPTTPTTPTG

SEQ ID NO: 98 - protein, 4 aa, Artificial Sequence
  Linker
  misc_feature (2)..(2): Xaa = any amino acid
  misc_feature (4)..(4): Xaa = any amino acid
PXPX

SEQ ID NO: 99 - protein, 4 aa, Artificial Sequence
  Linker
  misc_feature (1)..(1): Xaa = any amino acid
  misc_feature (3)..(3): Xaa = any amino acid
XPXP

SEQ ID NO: 100 - protein, 6 aa, Artificial Sequence
  Linker
  misc_feature (1)..(1): Xaa = any amino acid
  misc_feature (3)..(3): Xaa = any amino acid
  misc_feature (4)..(4): Xaa = any amino acid
  misc_feature (6)..(6): Xaa = any amino acid
XPXXPX

SEQ ID NO: 101 - protein, 6 aa, Artificial Sequence
  Linker
  misc_feature (1)..(1): Xaa = any amino acid
  misc_feature (2)..(2): Xaa = any amino acid
  misc_feature (4)..(4): Xaa = any amino acid
  misc_feature (5)..(5): Xaa = any amino acid
XXPXXP

SEQ ID NO: 102 - protein, 4 aa, Artificial Sequence
  Linker
  misc_feature (2)..(2): Xaa = Ser, Thr, Arg, Lys, Asp, Glu, Asn, Gln
  misc_feature (4)..(4): Xaa = Ser, Thr, Arg, Lys, Asp, Glu
PXPX

SEQ ID NO: 103 - protein, 4 aa, Artificial Sequence
  Linker
PSPS

SEQ ID NO: 104 - protein, 4 aa, Artificial Sequence
  Linker
PSPT

SEQ ID NO: 105 - protein, 4 aa, Artificial Sequence
  Linker
PSPR

SEQ ID NO: 106 - protein, 4 aa, Artificial Sequence
  Linker
PSPK

SEQ ID NO: 107 - protein, 4 aa, Artificial Sequence
  Linker
PSPD

SEQ ID NO: 108 - protein, 4 aa, Artificial Sequence
  Linker
PSPE

SEQ ID NO: 109 - protein, 4 aa, Artificial Sequence
  Linker
  misc_feature (2)..(2): Xaa = Ser, Glu
PXPT

SEQ ID NO: 110 - protein, 6 aa, Artificial Sequence
  Linker
PPSPTP

SEQ ID NO: 111 - protein, 6 aa, Artificial Sequence
  Linker
PPTPTP

SEQ ID NO: 112 - protein, 6 aa, Artificial Sequence
  Linker
PPSPSP

SEQ ID NO: 113 - protein, 6 aa, Artificial Sequence
  Linker
SPPPTP

SEQ ID NO: 114 - protein, 6 aa, Artificial Sequence
  Linker
SPTPPP

SEQ ID NO: 115 - protein, 6 aa, Artificial Sequence
  Linker
SPPPPP

SEQ ID NO: 116 - protein, 6 aa, Artificial Sequence
  Linker
SPTPTP

SEQ ID NO: 117 - protein, 6 aa, Artificial Sequence
  Linker
TPPPSP

SEQ ID NO: 118 - protein, 6 aa, Artificial Sequence
  Linker
TPSPPP

SEQ ID NO: 119 - protein, 6 aa, Artificial Sequence
  Linker
TPPPPP

SEQ ID NO: 120 - protein, 6 aa, Artificial Sequence
  Linker
TPSPSP

SEQ ID NO: 121 - protein, 8 aa, Artificial Sequence
  Linker
PSPTPEPT

SEQ ID NO: 122 - protein, 16 aa, Artificial Sequence
  Linker
PSPTPEPTPSPTPEPT

SEQ ID NO: 123 - protein, 8 aa, Artificial Sequence
  Linker
PEPTPSPT

SEQ ID NO: 124 - protein, 12 aa, Artificial Sequence
  Linker
PEPTPSPTPSPT

SEQ ID NO: 125 - protein, 4 aa, Artificial Sequence
  Linker
PEPT

SEQ ID NO: 126 - protein, 6 aa, Artificial Sequence
  Linker
SPPEPT

SEQ ID NO: 127 - protein, 6 aa, Artificial Sequence
  Linker
SPPSPT

SEQ ID NO: 128 - protein, 6 aa, Artificial Sequence
  Linker
PSPEPT

SEQ ID NO: 129 - protein, 6 aa, Artificial Sequence
  Linker
PSPSPT

SEQ ID NO: 130 - protein, 4 aa, Artificial Sequence
  Linker
SPSP

SEQ ID NO: 131 - protein, 6 aa, Artificial Sequence
  Linker
SPSPSP

SEQ ID NO: 132 - protein, 8 aa, Artificial Sequence
  Linker
SPSPSPSP

SEQ ID NO: 133 - protein, 12 aa, Artificial Sequence
  Linker
SPSPSPSPSPSP

SEQ ID NO: 134 - protein, 14 aa, Artificial Sequence
  Linker
SPSPSPSPSPSPSP

SEQ ID NO: 135 - protein, 16 aa, Artificial Sequence
  Linker
SPSPSPSPSPSPSPSP

SEQ ID NO: 136 - protein, 8 aa, Artificial Sequence
  Linker
PPPPPPPP

SEQ ID NO: 137 - protein, 9 aa, Artificial Sequence
  Linker
PPPPPPPPP

SEQ ID NO: 138 - protein, 10 aa, Artificial Sequence
  Linker
PPPPPPPPPP

SEQ ID NO: 139 - protein, 11 aa, Artificial Sequence
  Linker
PPPPPPPPPPP

SEQ ID NO: 140 - protein, 12 aa, Artificial Sequence
  Linker
PPPPPPPPPPPP

SEQ ID NO: 141 - protein, 13 aa, Artificial Sequence
  Linker
PPPPPPPPPPPPP

SEQ ID NO: 142 - protein, 14 aa, Artificial Sequence
  Linker
PPPPPPPPPPPPPP

SEQ ID NO: 143 - protein, 15 aa, Artificial Sequence
  Linker
PPPPPPPPPPPPPPP

SEQ ID NO: 144 - protein, 8 aa, Artificial Sequence
  Linker
PEPTPEPT

SEQ ID NO: 145 - protein, 12 aa, Artificial Sequence
  Linker
PEPTPEPTPEPT

SEQ ID NO: 146 - protein, 16 aa, Artificial Sequence
  Linker
PEPTPEPTPEPTPEPT

SEQ ID NO: 147 - protein, 8 aa, Artificial Sequence
  Linker
PSPTPSPT

SEQ ID NO: 148 - protein, 12 aa, Artificial Sequence
  Linker
PSPTPSPTPSPT

SEQ ID NO: 149 - protein, 16 aa, Artificial Sequence
  Linker
PSPTPSPTPSPTPSPT

SEQ ID NO: 150 - protein, 20 aa, Artificial Sequence
  Linker
PSPTPSPTPSPTPSPTPSPT

SEQ ID NO: 151 - protein, 6 aa, Artificial Sequence
  Linker
SPSSPS

SEQ ID NO: 152 - protein, 9 aa, Artificial Sequence
  Linker
SPSSPSSPS

SEQ ID NO: 153 - protein, 12 aa, Artificial Sequence
  Linker
SPSSPSSPSSPS

SEQ ID NO: 154 - protein, 15 aa, Artificial Sequence
  Linker
SPSSPSSPSSPSSPS

SEQ ID NO: 155 - protein, 6 aa, Artificial Sequence
  Linker
TPTTPT

SEQ ID NO: 156 - protein, 9 aa, Artificial Sequence
  Linker
TPTTPTTPT

SEQ ID NO: 157 - protein, 12 aa, Artificial Sequence
  Linker
TPTTPTTPTTPT

SEQ ID NO: 158 - protein, 15 aa, Artificial Sequence
  Linker
TPTTPTTPTTPTTPT

SEQ ID NO: 159 - protein, 16 aa, Artificial Sequence
  Linker
PEPTPRPTPEPTPRPT

SEQ ID NO: 160 - protein, 16 aa, Artificial Sequence
  Linker
PEPTPKPTPEPTPKPT

SEQ ID NO: 161 - protein, 16 aa, Artificial Sequence
  Linker
PEPTPQPTPEPTPQPT

SEQ ID NO: 162 - protein, 12 aa, Artificial Sequence
  Linker
PRPTPEPTPRPT

SEQ ID NO: 163 - protein, 12 aa, Artificial Sequence
  Linker
PKPTPEPTPKPT

SEQ ID NO: 164 - protein, 8 aa, Artificial Sequence
  Linker
PEPTPQPT

SEQ ID NO: 165 - protein, 12 aa, Artificial Sequence
  Linker
PEPTPQPTPEPT

SEQ ID NO: 166 - protein, 12 aa, Artificial Sequence
  Linker
TTPPTPTPTPTP

SEQ ID NO: 167 - protein, 14 aa, Artificial Sequence
  Linker
TTPTPPTPTPTPTP

SEQ ID NO: 168 - protein, 16 aa, Artificial Sequence
  Linker
TTPTPTPPTPTPTPTP

SEQ ID NO: 169 - protein, 33 aa, Artificial Sequence
  Linker
TPPTPPTPPTPPTPPTPPTPPTPPTPPTPPTPP

SEQ ID NO: 170 - protein, 13 aa, Artificial Sequence
  Linker
PPPPPPPPPPPPG

SEQ ID NO: 171 - protein, 15 aa, Artificial Sequence
  Linker
PPPPPPPPPPPPPPG

SEQ ID NO: 172 - protein, 16 aa, Artificial Sequence
  Linker
PPPPPPPPPPPPPPPG

SEQ ID NO: 173 - protein, 37 aa, Chaetomium thermophilum
CTTQKWGQCGGIGYTGCTNCVAGTTCTQLNPWYSQCL

SEQ ID NO: 174 - protein, 37 aa, Chaetomium thermophilum
CTTQKWGQCGGIGYTGCTNCVAGTTCTELNPWYSQCL

SEQ ID NO: 175 - protein, 36 aa, Mycothermus thermophilus
CASKWGQCGGQGWAGPTCCEAGSTCTRQNEWYSQCL

SEQ ID NO: 176 - protein, 37 aa, Ovatospora medusarum
CTAARWQQCGGIGYSGCKACASPWTCQKLNDWYHQCL

SEQ ID NO: 177 - protein, 37 aa, Thermothelomyces heterothallica
CTVAKWGQCGGQGYSGCTVCAAGSTCQKTNDYYSQCL

SEQ ID NO: 178 - protein, 38 aa, Ovatospora medusarum
CSVQAFGQCGGTGYSGCTQCADGYTCKDVSPPYYSQCV

SEQ ID NO: 179 - protein, 37 aa, Thielavia arenaria
CTVAKWGQCGGLGWTGCTTCAAGSTCNKANDFYSQCV

SEQ ID NO: 180 - protein, 37 aa, Ovatospora medusarum
CTVAKYGQCGGNNYSGCTTCAAGSTCSRTNEYYSQCV

SEQ ID NO: 181 - protein, 37 aa, Chaetomium thermophile
CVTQKWAQCGGNGFSGCRTCASGSTCQVLNEWYSQCL

SEQ ID NO: 182 - protein, 37 aa, Chaetomium thermophile
CTVAKWAQCGGIGYSGCTTCEAGSTCRRTNDYYSQCV

SEQ ID NO: 183 - protein, 36 aa, Ovatospora medusarum
CTAAQWQQCGGTNFNGCTTCAAGYNCKLINEYYSQC

SEQ ID NO: 184 - protein, 38 aa, Chaetomium thermophile
CTAQRYQQCGGNGYTGCTNCAAGSTCSAVSPPYYSQCL

SEQ ID NO: 185 - protein, 37 aa, Thermothelomyces heterothallica
CTAAQWAQCGGINFTGCTTCASPYKCNFINDYYSQCY

SEQ ID NO: 186 - protein, 37 aa, Thielavia species
CVAQKWAQCGGSGFTGCTTCASGSTCQKQNDFYSQCL

SEQ ID NO: 187 - protein, 38 aa, Thielavia arenaria
CTVQRYGQCGGQGYTGCTTCASGSTCTGVSAPYYYQCI

SEQ ID NO: 188 - protein, 37 aa, Thielavia arenaria
CAAAKYGQCDGKNWNGCKSCVAGTTCRYQNDYYSQCL

SEQ ID NO: 189 - protein, 37 aa, Ovatospora medusarum
CVAQKWAQCGGKGFTGCKNCVSGTTCQEQNQWYSQCL

SEQ ID NO: 190 - protein, 37 aa, Thielavia arenaria
CNVAQWQQCGGSTYTGCTQCASPYTCKNINTYYSQCQ

SEQ ID NO: 191 - protein, 37 aa, Chaetomium thermophile
CTAARWGQCGGIGYSGCTACASPWTCQRISDWYHQCL

SEQ ID NO: 192 - protein, 36 aa, Adineta vaga
CNNIYNQCGGNGWNGTTNCCSGLSCVYKNSSYSQCL

SEQ ID NO: 193 - protein, 37 aa, Chaetomium species
CVAQKWAQCGGKGFTGCTTCVSGTTCKEHHEWYSQCL

SEQ ID NO: 194 - protein, 37 aa, Achaetomium strumarium
CTAQRWSQCGGNGFTGCTTCVSGTTCQKQNDWYSQCL

SEQ ID NO: 195 - protein, 36 aa, Chaetomium thermophile
CTVPQWAQCGGVNYTGCTTCAPGYTCKYTNDYYSQC

SEQ ID NO: 196 - protein, 37 aa, Ovatospora medusarum
CVSQKWAQCGGNGYTGCTQCVSGTTCNKLNDWYSQCL

SEQ ID NO: 197 - protein, 37 aa, Chaetomium olivicolor
CTAQKWAQCGGSGFSGCTSCVSGTTCQKQNDWYSQCL

SEQ ID NO: 198 - protein, 37 aa, Taifanglania major
CVAQKWAQCGGNGFTGCTTCVSGTTCTKSNDWYSQCL

SEQ ID NO: 199 - protein, 37 aa, Trichocladium asperum
CFAQKWAQCGGNGFTGCTSCVSGTTCQKQNDWYSQCL

SEQ ID NO: 200 - protein, 37 aa, Ovatospora brasiliensis
CVAQKWAQCGGNGFSGCTTCVSGSTCQKQNDWYSQCL

SEQ ID NO: 201 - protein, 4 aa, Artificial Sequence
  Synthetic Construct
SQCL

SEQ ID NO: 202 - protein, 257 aa, Artificial sequence
  Synthetic construct
ASGSGQSTRYWDCCKPSCAWPGKAAVSQPVYSCDANFQRLSDFNVQSGCNGGSAYACADQTPWAVNDNLAYGFAATSIAGGSESSWCCACYALTFTSGPVAGKTMVVQSTSTGGDLGSNHFDIAMPGGGVGIFDGCSSQFGGLPGDRYGGISSRDQCDSFPAPLKPGCYWRFDWFQNADNPTVTFQQVQCPAEIVARSGCKRNDDSSFPVFTPPPPPPPGCTTQKWGQCGGIGWTGCTNCVAGTTCTQLNPWYSQCL